SEQUENCE LISTING (27 sequences)

SEQ ID NO: 1 - DNA, 228 bp, Artificial Sequence
  Chemically Synthesized, HBD
atgggcagcagccatcatcatcatcatcacagcagcggcctggtgccgcgcggcagccatatgaagttggcaaaaaaatactacgctgtaagaaaaggaagagttcccgggatctacaaaacctggaaagaggcggaagaacaggtaaaaggtttcccgggagccgagtacaaaagttttgaaagactggaagacgcgaaagcctatatggaaggaaagaacgagtga

SEQ ID NO: 2 - protein, 75 aa, Artificial Sequence
  Chemically Synthesized, HBD
MGSSHHHHHHSSGLVPRGSHMKLAKKYYAVRKGRVPGIYKTWKEAEEQVKGFPGAEYKSFERLEDAKAYMEGKNE

SEQ ID NO: 3 - DNA, 35 bp, Artificial Sequence
  Chemically Synthesized, HBD 5′ PCR primer
gggaattccatatgaagttggcaaaaaaatactac

SEQ ID NO: 4 - DNA, 39 bp, Artificial Sequence
  Chemically Synthesized, HBD 3′ PCR primer
gggaatggatcctcactcgttctttccttccatataggc

SEQ ID NO: 5 - DNA, 229 bp, Artificial Sequence
  Chemically Synthesized, HBD1-2S
gggaatggggtctcacgtctctgtgtccagttctgggctgatgctctcgttctttccttccatataggctttcgcgtcttccagtctttcaaaacttttgtactcggctcccgggaaaccttttacctgttcttccgcctctttccaggttttgtagatcccgggaactcttccttttcttacagcgtagtatttttttgccaacttcatatgcgagaccggaattccc

SEQ ID NO: 6 - DNA, 208 bp, Artificial Sequence
  Chemically Synthesized, HBD2 with a stop codon
gggaatggggtctcatcctctcactcgttctttccttccatataggctttcgcgtcttccagtctttcaaaacttttgtactcggctcccgggaaaccttttacctgttcttccgcctctttccaggttttgtagatcccgggaactcttccttttcttacagcgtagtatttttttgccaacttcatcgtccgagaccggaattccc

SEQ ID NO: 7 - DNA, 423 bp, Artificial Sequence
  Chemically Synthesized, DHBD
atgggcagcagccatcatcatcatcatcacagcagcggcctggtgccgcgcggcagccatatgaagttggcaaaaaaatactacgctgtaagaaaaggaagagttcccgggatctacaaaacctggaaagaggcggaagaacaggtaaaaggtttcccgggagccgagtacaaaagttttgaaagactggaagacgcgaaagcctatatggaaggaaagaacgagagcatcagcccagaactggacacagagacgatgaagttggcaaaaaaatactacgctgtaagaaaaggaagagttcccgggatctacaaaacctggaaagaggcggaagaacaggtaaaaggtttcccgggagccgagtacaaaagttttgaaagactggaagacgcgaaagcctatatggaaggaaagaacgagtga

SEQ ID NO: 8 - protein, 140 aa, Artificial Sequence
  Chemically Synthesized, DHBD
MGSSHHHHHHSSGLVPRGSHMKLAKKYYAVRKGRVPGIYKTWKEAEEQVKGFPGAEYKSFERLEDAKAYMEGKNESISPELDTETMKLAKKYYAVRKGRVPGIYKTWKEAEEQVKGFPGAEYKSFERLEDAKAYMEGKNE

SEQ ID NO: 9 - DNA, 229 bp, Artificial Sequence; identical to SEQ ID NO: 5
  Chemically Synthesized, HBD1-2S

SEQ ID NO: 10 - DNA, 221 bp, Artificial Sequence
  Chemically Synthesized, HBD1-1C
gggaatggggtctcagtccagttctgggctgatgctctcgttctttccttccatataggctttcgcgtcttccagtctttcaaaacttttgtactcggctcccgggaaaccttttacctgttcttccgcctctttccaggttttgtagatcccgggaactcttccttttcttacagcgtagtatttttttgccaacttcatcgtccgagaccggaattccc

SEQ ID NO: 11 - DNA, 208 bp, Artificial Sequence; identical to SEQ ID NO: 6
  Chemically Synthesized, HBD2-2S

SEQ ID NO: 12 - DNA, 217 bp, Artificial Sequence
  Chemically Synthesized, HBD3 with stop codon
gggaatggggtctcatcctctcactcgttctttccttccatataggctttcgcgtcttccagtctttcaaaacttttgtactcggctcccgggaaaccttttacctgttcttccgcctctttccaggttttgtagatcccgggaactcttccttttcttacagcgtagtatttttttgccaacttcatcgtctctgtgtcccgagaccggaattccc

SEQ ID NO: 13 - DNA, 618 bp, Artificial Sequence
  Chemically Synthesized, THBD
atgggcagcagccatcatcatcatcatcacagcagcggcctggtgccgcgcggcagccatatgaagttggcaaaaaaatactacgctgtaagaaaaggaagagttcccgggatctacaaaacctggaaagaggcggaagaacaggtaaaaggtttcccgggagccgagtacaaaagttttgaaagactggaagacgcgaaagcctatatggaaggaaagaacgagagcatcagcccagaactggacacagagacgatgaagttggcaaaaaaatactacgctgtaagaaaaggaagagttcccgggatctacaaaacctggaaagaggcggaagaacaggtaaaaggtttcccgggagccgagtacaaaagttttgaaagactggaagacgcgaaagcctatatggaaggaaagaacgagagcatcagcccagaactggacacagagacgatgaagttggcaaaaaaatactacgctgtaagaaaaggaagagttcccgggatctacaaaacctggaaagaggcggaagaacaggtaaaaggtttcccgggagccgagtacaaaagttttgaaagactggaagacgcgaaagcctatatggaaggaaagaacgagtga

SEQ ID NO: 14 - protein, 205 aa, Artificial Sequence
  Chemically Synthesized, THBD
MGSSHHHHHHSSGLVPRGSHMKLAKKYYAVRKGRVPGIYKTWKEAEEQVKGFPGAEYKSFERLEDAKAYMEGKNESISPELDTETMKLAKKYYAVRKGRVPGIYKTWKEAEEQVKGFPGAEYKSFERLEDAKAYMEGKNESISPELDTETMKLAKKYYAVRKGRVPGIYKTWKEAEEQVKGFPGAEYKSFERLEDAKAYMEGKNE

SEQ ID NO: 15 - DNA, 618 bp, Artificial Sequence
  Chemically Synthesized, THBD-1C
atgggcagcagccatcatcatcatcatcacagcagcggcctggtgccgcgcggcagccatatgaagttggcaaaaaaatactacgctgtaagaaaaggaagagttcccgggatctacaaaacctggaaagaggcggaagaacaggtaaaaggtttcccgggagccgagtacaaaagttttgaaagactggaagacgcgaaagcctatatggaaggaaagaacgagagcatctgtccagaactggacacagagacgatgaagttggcaaaaaaatactacgctgtaagaaaaggaagagttcccgggatctacaaaacctggaaagaggcggaagaacaggtaaaaggtttcccgggagccgagtacaaaagttttgaaagactggaagacgcgaaagcctatatggaaggaaagaacgagagcatcagcccagaactggacacagagacgatgaagttggcaaaaaaatactacgctgtaagaaaaggaagagttcccgggatctacaaaacctggaaagaggcggaagaacaggtaaaaggtttcccgggagccgagtacaaaagttttgaaagactggaagacgcgaaagcctatatggaaggaaagaacgagtga

SEQ ID NO: 16 - protein, 185 aa, Artificial Sequence
  Chemically Synthesized, THBD-1C
MKLAKKYYAVRKGRVPGIYKTWKEAEEQVKGFPGAEYKSFERLEDAKAYMEGKNESICPELDTETMKLAKKYYAVRKGRVPGIYKTWKEAEEQVKGFPGAEYKSFERLEDAKAYMEGKNESISPELDTETMKLAKKYYAVRKGRVPGIYKTWKEAEEQVKGFPGAEYKSFERLEDAKAYMEGKNE

SEQ ID NO: 17 - DNA, 229 bp, Artificial Sequence
  Chemically Synthesized, HBD linked to a 2C linker
gggaatggggtctcacgtctctgtgtccagttctggacagatgcactcgttctttccttccatataggctttcgcgtcttccagtctttcaaaacttttgtactcggctcccgggaaaccttttacctgttcttccgcctctttccaggttttgtagatcccgggaactcttccttttcttacagcgtagtatttttttgccaacttcatatgcgagaccggaattccc

SEQ ID NO: 18 - DNA, 423 bp, Artificial Sequence
  Chemically Synthesized, DHBD-2C
atgggcagcagccatcatcatcatcatcacagcagcggcctggtgccgcgcggcagccatatgaagttggcaaaaaaatactacgctgtaagaaaaggaagagttcccgggatctacaaaacctggaaagaggcggaagaacaggtaaaaggtttcccgggagccgagtacaaaagttttgaaagactggaagacgcgaaagcctatatggaaggaaagaacgagtgcatctgtccagaactggacacagagacgatgaagttggcaaaaaaatactacgctgtaagaaaaggaagagttcccgggatctacaaaacctggaaagaggcggaagaacaggtaaaaggtttcccgggagccgagtacaaaagttttgaaagactggaagacgcgaaagcctatatggaaggaaagaacgagtga

SEQ ID NO: 19 - protein, 140 aa, Artificial Sequence
  Chemically Synthesized, DHBD-2C
MGSSHHHHHHSSGLVPRGSHMKLAKKYYAVRKGRVPGIYKTWKEAEEQVKGFPGAEYKSFERLEDAKAYMEGKNECICPELDTETMKLAKKYYAVRKGRVPGIYKTWKEAEEQVKGFPGAEYKSFERLEDAKAYMEGKNE

SEQ ID NO: 20 - protein, 55 aa, Artificial Sequence
  Chemically Synthesized, hybrid binding domain
MKLAKKYYAVRKGRVPGIYKTWKEAEEQVKGFPGAEYKSFERLEDAKAYMEGKNE

SEQ ID NO: 21 - protein, 10 aa, Artificial Sequence
  Chemically Synthesized, Linker-S
SISPELDTET

SEQ ID NO: 22 - protein, 10 aa, Artificial Sequence
  Chemically Synthesized, Linker-C
SICPELDTET

SEQ ID NO: 23 - DNA, 47 bp, Artificial Sequence
  Chemically Synthesized, 21 bp DNA-RNA hairpin used for the SPR analyses
tcaacgtttcggaccgtatttcttttgaaauacgguccgaaacguug

SEQ ID NO: 24 - DNA, 21 bp, Artificial Sequence
  Chemically Synthesized, DNA portion of the 21 bp DNA-RNA hairpin used for the SPR analyses
caacgtttcggaccgtatttc

SEQ ID NO: 25 - RNA, 21 nt, Artificial Sequence
  Chemically Synthesized, RNA portion of the 21 bp DNA-RNA hairpin used for the SPR analyses
gaaauacgguccgaaacguug

SEQ ID NO: 26 - DNA, 11 bp, Artificial Sequence
  Chemically Synthesized, , BsaI cleavage site
  misc_feature (1)..(5): any nuclotide
nnnnngagacc

SEQ ID NO: 27 - DNA, 11 bp, Artificial Sequence
  Chemincally Synthesized, BsaI cleavage site
  misc_feature (7)..(11): any nucleotide
ggtctcnnnnn

(19) United States
(12) Patent Application Publication
(10) Pub. No.: US 2023/0175040 A1
(43) Pub. Date: Jun. 8, 2023

(54) Epiprinter Technology and Methods of Use for Detecting Biomolecules

(71) Applicant: Temple University-Of The Commonwealth System of Higher Education, Philadelphia, PA (US)

(72) Inventors: Allen NICHOLSON, Moorestown, NJ (US); Alex STOPAR, Philadelphia, PA (US); Matteo CASTRONOVO, Leeds (GB)

(21) Appl. No.: 17/997,572

(22) PCT Filed: Apr. 30, 2021
(86) PCT No.: PCT/US2021/030140

§ 371 Date: Oct. 31, 2022

Related U.S. Application Data
(60) Provisional application No. 63/017,819, filed on Apr. 30, 2020.

(51) Int. Cl. C12Q 1/6816; C12Q 1/6888; C12Q 1/6876; C12Q 1/6837

(52) U.S. Cl. CPC C12Q 1/6816; C12Q 1/6888; C12Q 1/6876; C12Q 1/6837

(57) ABSTRACT

The invention relates to compositions and methods to detect hybrids with high sensitivity and selectivity in order to assess cell, tissue and organism function in both health and disease. The invention described herein, termed epiprinter, is a functionally versatile molecular entity that can detect RNA-DNA hybrids with potentially high sensitivity and selectivity.

### CROSS REFERENCE TO RELATED APPLICATIONS

This application claims priority to U.S. Provisional Application No. 63/017,819, filed Apr. 30, 2020, which is hereby incorporated by reference herein in its entirety.

### BACKGROUND

RNA detection technologies are widely used in laboratories and clinics for disease diagnosis and measurement of gene expression. RNA detection technologies also can identify pathogens such as RNA viruses in humans, animals, plants, food, and water, as well as in insect and animal vectors that transmit disease agents. There is a growing, yet still unmet need to detect RNA with high sensitivity and accuracy, in a cost-effective manner, and in diverse, resource-limited settings.

RNA-DNA hybrids are formed by the association of an RNA with a DNA of complementary sequence, and participate in diverse cellular processes. There is an important need to detect hybrids with high sensitivity and selectivity in order to assess cell, tissue and organism function in both health and disease.

DNA-RNA hybrids are conserved structures that are essential participants in diverse cellular processes including RNA transcription, DNA replication, DNA repair, DNA recombination, telomere maintenance, and the replication of retroviruses including HIV (Niehrs and Luke, 2020, Nature Reviews Molecular Cell Biology, 21:167-178; Tian et al., 2018, Proceedings of the National Academy of Sciences, 115:507-512). R-loops, which are three-stranded cellular structures consisting of a DNA-RNA hybrid along with a displaced DNA strand, occur in the nucleus and nucleolus, as well as in the cytoplasm (reviewed by (Vanoosthuyse, 2018, Non-Coding RNA, 4:9). Although the occurrence of R-loops in the cytoplasm is not well understood, possible explanations include R-loops present in the DNA of the mitochondria that are localized to the cytoplasm, and also probable specificity issues with the currently-used anti-DNA-RNA antibody (Vanoosthuyse, 2018, Non-Coding RNA, 4:9). The ability to detect R-loops and other hybrids within cells with high sensitivity and specificity can provide fundamental information on the functions and dynamics of R-loops, as well as other DNA-RNA hybrid structures that occur in normal and disease states of cells, including viral infection and cancer.

Detecting R-loops and mapping their spatial and temporal distributions have primarily involved immunofluorescence, and/or immunoprecipitation/sequencing approaches, with each approach using the hybrid-specific S9.6 antibody that was generated using a synthetic DNA-RNA hybrid as antigen (Boguslawski et al. 1986, Journal of Immunological Methods, 89:123-130). While the S9.6 antibody has been productively used in a number of studies (reviewed in (Vanoosthuyse, 2018, Non-Coding RNA, 4:9)), its action is sensitive to the exact method of cell fixation (Skourti-Stathaki et al., 2014, Nature 516:436-439), and it also exhibits variability in binding affinity that is dependent upon hybrid base-pair sequence (König et al., 2017, PLOS ONE 12 (6): e0178875). These limitations can introduce bias in the detecting cellular hybrid structures. Also, the S9.6 antibody has an appreciable affinity for double-stranded(ds) RNA (Phillips et al., 2013, Journal of Molecular Recognition, 26: 376-381; Hartono et al., 2018, Journal of Molecular Biology, 430: 272-284), and can provide false positive outputs due to dsRNA binding. The off-target dsRNA binding may contribute to the cytoplasmic signal (see above). Alternative approaches to detecting hybrids have used a single copy of the hybrid-binding domain (HBD) of human RNase H fused to Green Fluorescent Protein (GFP). This construct was expressed in situ to identify R-loops in yeast cells and in mammalian cells (Bhatia et al., 2014, Nature, 511: 362-365). A catalytically inactive form of RNase H that retains the ability to bind RNA-DNA hybrids was used to isolate R-loop structures from mammalian cells for sequencing and mapping analyses (Chen et al., 2017, Molecular Cell, 68:745-757.e5).

The emergence and spread of vector-borne viral diseases into new geographic areas are having an increasing impact on human populations. This is especially underscored by the recent spread of Zika virus (ZIKV) disease into the Caribbean and into Florida. The ongoing expansion of ZIKV and other mosquito-borne viral diseases, including Dengue and Chikungunya, is spurring the development of methods for the rapid, sensitive and accurate detection of viruses that can be easily used in resource-limited settings. Rapid identification of pathogenic viruses in patients in remote settings as well as the clinic, and the rapid field surveillance of insect vectors can enable earlier deployment of vaccines and treatments, and allow more efficient, targeted control of disease vector populations. Current disease agent detection methods typically involve enzymatic amplification of the nucleic acid, with reporter labels providing fluorescent or colorimetric outputs. Also, multiplexed amplification approaches can interrogate samples for over twenty different viruses. However, for these enzyme-based detection methods, false positive or negative outputs are a persistent problem, and the enzymes and reporter labels add to assay complexity and cost. These considerations are spurring the development of amplification- and enzyme-free methods for RNA detection.

Accordingly, there is a need for improved systems and methods that permit rapid, sensitive, and accurate detection of RNA and RNA-DNA hybrids. The present invention fulfills this need.

### SUMMARY OF THE INVENTION

In one embodiment, the invention relates to a composition comprising an epiprinter molecule comprising at least one RNA:DNA hybridization domain (HBD) and at least one reactive moiety. In one embodiment, the epiprinter molecule comprises two HBDs. In one embodiment, the epiprinter molecule comprises three HBDs.

In one embodiment, the epiprinter molecule comprises a sequence comprising at least 75% identity to SEQ ID NO:2, SEQ ID NO:8, SEQ ID NO:14, SEQ ID NO:16, or SEQ ID NO:19.

In one embodiment, the epiprinter molecule comprises two or more reactive moieties.

In one embodiment, the reactive moiety is selected from the group consisting of an azide group, an alkyne group, a sulfur group, and a Bicyclo[6.1.0] nonyne (BCN) groups.

In one embodiment, the reactive moiety is capable of undergoing a click chemistry reaction.

In one embodiment, the reactive moiety is a side group of an amino-acid residue included in a linker sequence between two HBDs of the epiprinter molecule.

In one embodiment, the invention relates to a composition comprising a nucleotide sequence encoding an epiprinter molecule comprising at least one RNA:DNA hybridization domain (HBD) and at least one reactive moiety, or a fragment thereof. In one embodiment, the epiprinter molecule comprises two HBDs. In one embodiment, the epiprinter molecule comprises three HBDs.

In one embodiment, the nucleotide sequence encodes a sequence comprising at least 75% identity to SEQ ID NO:2, SEQ ID NO:8, SEQ ID NO:14, SEQ ID NO:16, or SEQ ID NO:19, or a fragment thereof comprising at least 56 amino acid residues.

In one embodiment, the nucleotide sequence has at least 75% identity to SEQ ID NO:1, SEQ ID NO:5; SEQ ID NO:6, SEQ ID NO:7, SEQ ID NO:9, SEQ ID NO:10, SEQ ID NO:11, SEQ ID NO:12, SEQ ID NO:13, SEQ ID NO:15, SEQ ID NO:17, or SEQ ID NO:18.

In one embodiment, the nucleotide sequence comprises a fragment comprising at least 168 nucleotides of SEQ ID NO:1, SEQ ID NO:5; SEQ ID NO:6, SEQ ID NO:7, SEQ ID NO:9, SEQ ID NO:10, SEQ ID NO:11, SEQ ID NO:12, SEQ ID NO:15, SEQ ID NO:17, or SEQ ID NO:18 in the sense or anti-sense direction.

In one embodiment, the invention relates to a composition comprising an epitape molecule, wherein the epitape molecule comprises a DNA nanostructure comprising a structural strand of the M13 phage, mixture of oligonucleotide staples, and at least one probe strand comprising a sequence complementary to a target nucleic acid molecule of interest and at least one reactive site.

In one embodiment, the at least one probe strand is selected from the group consisting of RNA and DNA.

In one embodiment, the epitape molecule comprises at least two probe strands which together form a probe site. In one embodiment, the epitape molecule comprises at least four probe strands which together form at least two probe sites.

In one embodiment, the at least one reactive site comprises at least one terminal alkyne on at least one probe strand. In one embodiment, the at least one reactive site comprises at least one BCN group.

In one embodiment, the epitape comprises at least two probe sites and the at least one reactive site comprises at least one terminal alkyne or BCN group on the probe strand of the second probe site.

In one embodiment, the epitape molecule further comprises at least one barcode strand, wherein the barcode strand comprises a sequence that forms structure that is detectable upon translocation through a nanopore.

In one embodiment, the structure is selected from the group consisting of a hairpin, a dumbbell and a bulge.

In one embodiment, the epitape molecule further comprises at least one barcode strand, wherein the barcode strand comprises at least one reactive site for interaction with a molecule comprising a reactive moiety, wherein the reaction generates a structure that is detectable upon translocation through a nanopore. In one embodiment, the reactive site allows click-chemistry, or a sulfhydryl-maleimide reaction with a small protein or a molecular polymer comprising an appropriate reactive moiety.

In one embodiment, the invention relates to a system for detection a molecule of interest comprising at least one epiprinter molecule comprising at least one RNA:DNA hybridization domain (HBD) and at least one reactive moiety, at least one epitape molecule comprising a DNA nanostructure comprising a structural strand of the M13 phage, mixture of oligonucleotide staples, and at least one probe strand comprising a sequence complementary to a target nucleic acid molecule of interest and at least one reactive site, wherein the epitape molecule comprises a nucleotide sequence complementary to a nucleotide sequence of the target of interest, and a nanopore detection system comprising a first reservoir containing an electrically conductive aqueous solution; an electrode disposed within the first reservoir in electrical contact with the electrically conductive aqueous solution; a second reservoir containing an electrically conductive aqueous solution; another electrode disposed within the second reservoir and in electrical contact with the electrically conductive aqueous solution; and a membrane separating the two reservoirs, the membrane having a pore through which the epiprinter/epitape complex can pass.

In one embodiment, the invention relates to a method for detecting the presence of target molecule of interest, the method comprising the steps of: a) contacting the target molecule of interest with an epitape molecule comprising a probe site comprising a nucleotide sequence which is complementary to a region of the target molecule of interest; b) contacting the target molecule:epitape complex with an epiprinter molecule; c) allowing a cycloaddition reaction to occur between the epiprinter molecule and the epitape molecule; and d) detecting the epiprinter:epitape complex using a nanopore system.

In one embodiment, the target molecule of interest is a viral nucleic acid molecule, a bacterial nucleic acid molecule, a microRNA molecule, an mRNA molecule, an alternatively spliced mRNA molecule, a nucleic acid molecule harboring a disease-associated mutation, and a biomarker associated with a disease or disorder.

In one embodiment, the invention relates to a method for detecting the presence of an RNA molecule of interest, the method comprising the steps of: a) contacting an RNA molecule of interest with an epitape molecule comprising a probe site comprising a DNA sequence which is complementary to a region of the RNA molecule of interest; b) contacting the RNA molecule:epitape complex with an epiprinter molecule; c) allowing a cycloaddition reaction to occur between the epiprinter molecule and the epitape molecule; and d) detecting the epiprinter:epitape complex using a nanopore system.

In one embodiment, the invention relates to a method for detecting the presence of a DNA molecule of interest, the method comprising the steps of: a) contacting a DNA molecule of interest with an epitape molecule comprising a probe site comprising a RNA sequence which is complementary to a region of the DNA molecule of interest; b) contacting the DNA molecule:epitape complex with an epiprinter molecule; c) allowing a cycloaddition reaction to occur between the epiprinter molecule and the epitape molecule; and d) detecting the epiprinter:epitape complex using a nanopore system.

In one embodiment, the target molecule of interest is a protein, a peptide, a chemical compound, a small molecule, a drug or a metabolite.

In one embodiment, the invention relates to a method for indirectly detecting the presence of the target molecule of interest, the method comprising the steps of: a) contacting a target molecule of interest with a mediator complex comprising a molecule which binds specifically to the target and a nucleic acid molecule which is released upon binding of the mediator complex to the target; b) contacting the nucleic acid molecule which was released upon binding of the mediator complex to the target with an epitape molecule comprising a probe site comprising a RNA sequence which is complementary to a region of the nucleic acid molecule which was released upon binding of the mediator complex; c) contacting the nucleic acid molecule:epitape complex with an epiprinter molecule; d) allowing a cycloaddition reaction to occur between the epiprinter molecule and the epitape molecule; and e) detecting the epiprinter:epitape complex using a nanopore system.

In one embodiment, the mediator complex comprises an antibody, antibody fragment or aptamer specific for binding to a target molecule of interest.

In one embodiment, the invention relates to a method of diagnosing a mammal with a disease or disorder, the method comprising the steps of: a) detecting the presence of a nucleic acid biomarker of interest in a sample obtained from the mammal, wherein the presence of the nucleic acid biomarker of interest is associated with the disease or disorder, the method of detecting comprising: i) contacting the sample with an epitape comprising a probe site with a probe strand comprising a nucleotide sequence complementary to a region of the biomarker of interest, wherein when the biomarker of interest is an RNA molecule, the probe strand comprises a DNA molecule, wherein when the biomarker of interest is a DNA molecule, the probe strand comprises an RNA molecule, such that the biomarker of interest hybridizes to the probe site of the epitape molecule forming an RNA:DNA hybrid; ii) contacting the hybridized epitape: biomarker of interest with an epiprinter, whereby the epiprinter undergoes a cycloaddition reaction with the epitape molecule, becoming covalently linked to the epitape molecule; iii) translocating the covalently linked epiprinter-epitape molecule through a nanopore, whereby the covalently linked epiprinter-epitape molecule transiently blocks the electrical signal as it passes through the nanopore; and iv) measuring the electrical current in the nanopore system, wherein a decrease in electrical current as compared to a control indicates the presence of the biomarker of interest in the sample; and b) diagnosing the mammal with the disease or disorder when the presence of the associated biomarker is detected.

In one embodiment, the target molecule of interest is a viral nucleic acid molecule, a bacterial nucleic acid molecule, a microRNA molecule, an mRNA molecule, an alternatively spliced mRNA molecule, a nucleic acid molecule harboring a disease-associated mutation, and a biomarker associated with a disease or disorder.

In one embodiment, the invention relates to an isolated nucleic acid molecule comprising a nucleotide sequence encoding an RNA:DNA hybrid binding molecule comprising at least one RNA:DNA hybrid binding domain. In one embodiment, the nucleic acid molecule comprises a sequence having at least 75% identity to SEQ ID NO:1, SEQ ID NO:5; SEQ ID NO:6, SEQ ID NO:7, SEQ ID NO:9, SEQ ID NO:10, SEQ ID NO:11, SEQ ID NO:12, SEQ ID NO:13, SEQ ID NO:15, SEQ ID NO:17, or SEQ ID NO:18. In one embodiment, the nucleic acid molecule comprises a sequence comprising at least 168 consecutive nucleotides of SEQ ID NO:1, SEQ ID NO:5; SEQ ID NO:6, SEQ ID NO:7, SEQ ID NO:9, SEQ ID NO:10, SEQ ID NO:11, SEQ ID NO:12, SEQ ID NO:15, SEQ ID NO:17, or SEQ ID NO:18. In one embodiment, the nucleic acid molecule comprises SEQ ID NO:1, SEQ ID NO:5, SEQ ID NO:7, SEQ ID NO:13, SEQ ID NO:15 or SEQ ID NO:18.

In one embodiment, the invention relates to an RNA:DNA hybrid binding molecule comprising at least one RNA:DNA hybrid binding domain, wherein the molecule comprises an amino acid sequence of SEQ ID NO:2, SEQ ID NO:6, SEQ ID NO:8, SEQ ID NO:14, SEQ ID NO:16 or SEQ ID NO:19.

In one embodiment, the invention relates to a method of binding at least one RNA:DNA hybrid molecule, the method comprising, contacting a sample comprising at least one RNA:DNA hybrid molecule with an RNA:DNA hybrid binding molecule comprising at least one RNA:DNA hybrid binding domain, wherein the molecule comprises an amino acid sequence of SEQ ID NO:2, SEQ ID NO:6, SEQ ID NO:8, SEQ ID NO:14, SEQ ID NO:16 or SEQ ID NO:19

In one embodiment, the invention relates to a method of binding at least one RNA:DNA hybrid molecule, the method comprising, contacting a sample comprising at least one RNA:DNA hybrid molecule with an isolated nucleic acid molecule comprising a nucleotide sequence encoding an RNA:DNA hybrid binding molecule comprising at least one RNA:DNA hybrid binding domain. In one embodiment, the nucleic acid molecule comprises a sequence having at least 75% identity to SEQ ID NO:1, SEQ ID NO:5; SEQ ID NO:6, SEQ ID NO:7, SEQ ID NO:9, SEQ ID NO:10, SEQ ID NO:11, SEQ ID NO:12, SEQ ID NO:13, SEQ ID NO:15, SEQ ID NO:17, or SEQ ID NO:18. In one embodiment, the nucleic acid molecule comprises a sequence comprising at least 168 consecutive nucleotides of SEQ ID NO:1, SEQ ID NO:5; SEQ ID NO:6, SEQ ID NO:7, SEQ ID NO:9, SEQ ID NO:10, SEQ ID NO:11, SEQ ID NO:12, SEQ ID NO:15, SEQ ID NO:17, or SEQ ID NO:18. In one embodiment, the nucleic acid molecule comprises SEQ ID NO:1, SEQ ID NO:5, SEQ ID NO:7, SEQ ID NO:13, SEQ ID NO:15 or SEQ ID NO:18.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts an overall scheme for RNA detection by epitape-epiprinter technology. Epiprinter and Proto-epitape details are provided in FIGS. 2 and 3, respectively. The click-created covalent bond is shown, and the proposed signature current change caused by epitape-epiprinter translocation through the solid-state (quartz) nanopore is also shown.

FIG. 2A through FIG. 2E depict a diagram of the v.1 epiprinter, and covalent attachment of the v.1 epiprinter to an RNA-DNA hybrid. FIG. 2A depicts the structural and functional features of the v.1 epiprinter as a dimeric HBD (DHBD). The two cysteines (C) in the linker within the DHBD-2C protein each carry alkyl azide groups, attached via maleimide conjugation. FIG. 2B depicts an SDS-PAGE analysis of purified recombinant DHBD-2C (14 kDa; the HBD is from Thermotoga maritima RNase H1), with MW markers. FIG. 2C depicts a phosphorimage of a gel mobility shift assay of the DHBD-2C and HBD using a ³²P-labeled 20 bp RNA-DNA hybrid (“RD”). FIG. 2D depicts a scheme for v.1 epiprinter reaction with the 20 bp RNA-DNA* hybrid, with the DNA* strand synthesized to carry alkynyl modifications of the 5-carbons of two internal dU residues. FIG. 2E depicts a phosphorimage of denaturing polyacrylamide gel analysis of the v.1 epiprinter reaction with the 20 bp hybrid. The reaction time was 30 minutes (22° C.). The two slower-migrating species carry one or two attached v.1 epiprinters (denoted “Epi”), reflecting the two attachment sites on the DNA strand of the hybrid. The 2nd-order rate constant for the Cu(I)-catalyzed azide-alkyne coupling reaction is ˜10-200 M⁻¹ s⁻¹ (McKay and Finn 2014).

FIG. 3 depicts exemplary proto-epitape structural and functional features. The linear structure on top indicates the placement of barcode elements (digital 1/0 readout), employing DNA “dumbbell” secondary structures providing the nanopore signal. Alkynyl groups on the DNA strands of probe site 2 are shown. The manner of binding of RNA is similar to that described previously (Ke et al. 2008). The inset diagram shows the interaction of scaffold and staples, with DNA crossovers that stabilize the proto-epitape structure.

FIG. 4 depicts a schematic representation of the THBD protein structure (188 a.a., 21.7 kDa), containing three copies of the HBD, conjugated with fluorophore Alexa Fluor™ 647 (A647). On top, the sequence of the N-terminal Hybrid Binding Domain (HBD, 55 a.a.) originating from Thermotoga maritima RNase H1 enzyme. In the middle, the sequence of the two linker regions (10 a.a.) that differ in composition at the 3rd residue: LNK-C bears a cysteine and LNK-S bears a serine. Maleimide-sulfhydryl chemistry was used for the stoichiometric conjugation of the Maleimide-A647 with the THBD.

FIG. 5A and FIG. 5B depict exemplary experimental results demonstrating detection of DNA-RNA hybrids in HeLa cells by the THBD-A647. FIG. 5A depicts fluorescence confocal-microscopy of HeLa cells immuno-stained with THBD-A647 and S9.6-Cy3. FIG. 5B depicts cells that were treated same as FIG. 5A, but also were stained with Hoechst dye to visualize the nuclei.

FIG. 6 depicts the structure and sequence of the 47 nt chimeric oligonucleotide that, following intramolecular hybridization, forms a (21 bp) DNA-RNA hairpin used for the SPR analyses. The biotin moiety attached to the 5′-end allowed the stable attachment of the hybrid to the neutravidin immobilized on the chip surface.

FIG. 7A and FIG. 7B depict exemplary experimental results demonstrating SPR determination of HBD affinity for DNA-RNA hybrid. In FIG. 7A, the sensorgram shows the binding response of the 10 RU DNA-RNA hybrid surface to injected HBD at 0.117, 0.234, 0.469, 0.938, 1.875, 3.75, 7.5, 15, 30, and 60 μM concentrations. FIG. 7B is a plot of the binding response at equilibrium as a function of HBD concentration. A K_{D} (dissociation constant) of 8.14 μM was determined from data that was fitted to a 1:1 interaction model (black line, chi-square 0.425 RU) using the global data analysis tool available in the BiaEvaluation 3.2 software.

FIG. 8A and FIG. 8B depict exemplary experimental results demonstrating the DHBD affinity for the 21 bp DNA-RNA hybrid, using SPR. In FIG. 8A, the sensorgram shows the binding response of the DNA-RNA hybrid surface (10 RU) to the DHBD at 0.488, 0.977, 1.953, 3.906, 7.813, 15.625, 31.25, 62.5, 125 and 250 nM concentrations. The colored lines are the raw data, while the black lines show the kinetic fittings determined with the BIAevaluation 3.2 software, using a 1:1 binding model. FIG. 8B displays a plot of the binding response at equilibrium as a function of the DHBD concentration; A K_{D} of 21.3 nM was obtained from data fitting to a 1:1 interaction model (black line, chi-square 0.413 RU) using the global data analysis tool available in the BiaEvaluation 3.2 software.

FIG. 9A and FIG. 9B depict exemplary experimental results demonstrating the THBD affinity for the 21 bp DNA-RNA hybrid using SPR. FIG. 9A displays the sensorgram showing the binding response of the surface-immobilized DNA-RNA hybrid (10 RU) to the THBD at 0.003, 0.012, 0.049, 0.195, 0.781, 3.125, 12.5 and 50 nM concentrations. The colored lines are the raw data, while the black lines are the kinetic fittings, using the BIAevaluation 3.2 software and applying a 1:1 binding model. FIG. 9B shows a plot of the binding response at equilibrium as a function of THBD concentration. A K_{D} of 375 pM was obtained from data fitting to a 1:1 interaction model (black line, chi-square 0.12 RU) using the global data analysis tool available in BiaEvaluation 3.2 software.

FIG. 10A and FIG. 10B depict exemplary experimental results demonstrating modeling and analysis of the THBD interaction with 21 bp DNA-RNA hybrid. FIG. 10A shows the structure of three copies of human HBD complexed with a 12 bp DNA-RNA hybrid (reproduced from PDB ID: 3BSU (Nowotny et al., 2008, EMBO J. 27: 1172-1181)). The black dashed arrow indicates a possible path for a 10 amino acid linker connecting the C-terminal of the HBD on the top (red) to the N-terminal of the neighboring HBD at the center (yellow). FIG. 10B displays a schematic model of the interaction between the 21 bp DNA-RNA hybrid and the single HBD or the THBD. The 21 bp hybrid can accommodate up to six copies of HBD, or one copy of the THBD.

FIG. 11 depicts the structure of the Thermotoga maritima HBD complexed with a 12 bp DNA-RNA hybrid, as determined using homology modeling. The HBD structure spans 9 base pairs of the hybrid.

FIG. 12 depicts the design and assembly of the recombinant HBD protein.

FIG. 13 depicts the design and assembly of the recombinant DHBD protein.

FIG. 14 depicts the design and assembly of the recombinant THBD and THBD-1C proteins.

FIG. 15A through FIG. 15C depicts the sequence, schematic structure, production and purification of recombinant single HBD (SHBD), double HBD (DHBD), and triple HBD (THBD) proteins. FIG. 15A depicts amino acid sequences of the HBD and the modified linker (see also Results) from T. maritima RNase H1. FIG. 15B depicts the domain organization of the SHBD, DHBD and THBD proteins. The 10 amino acid linker (grey) connects adjacent HBDs (green) in the two multi-HBD proteins. FIG. 15C depicts a gel electrophoretic analysis of the production and purification of recombinant SHBD, DHBD and THBD proteins. Shown is a Coomassie Blue stained SDS polyacrylamide gel, showing overexpression in E. coli of His-tagged SHBD (S), DHBD (D), and THBD (T) proteins. Lanes 1-3 display total protein profile prior to IPTG induction (−Ind). Lanes 4-6 display total protein profile after induction and incubation for 4 hr at 37° C. (+Ind). Lanes 8-10 display His-tag removed and purified proteins. Each protein contains three additional N-terminal amino acids, representing the remaining portion of the thrombin recognition sequence. The three proteins exhibit molecular masses in agreement with their predicted masses (indicated on the right). Lane 7 shows protein MW markers (numbers in kDa).

FIG. 16 depicts an adaptation of pET-15b plasmid for use with BsaI in Golden Gate assembly. To adapt pET-15b for this usage, the single BsaI site in the b-lactamase gene was removed and a BsaI site introduced in the multi-cloning sequence. The BsaI sequence in the b-lactamase gene was altered by creating a single nucleotide mutation at position 4781, using the Q5 mutagenesis kit (NEB) and specific primers. The mutation did not alter the beta-lactamase amino acid sequence. The mutation was verified by DNA sequencing, and E. coli cells transformed with the altered plasmid retained the ability to grow on ampicillin-containing LB agar plates. The plasmid then was further engineered to carry a short synthetic sequence containing a BsaI site, that was inserted between the NdeI and BamHI sites in the multicloning sequence. This step also used the Q5 mutagenesis kit. The sequence of the inserted region was verified by DNA sequencing. The resultant plasmid, pET15bGGA, was used to clone the DHBD and THBD coding sequences.

FIG. 17A through FIG. 17D depict gel electrophoretic mobility shift analyses of RNA-DNA hybrid binding to SHBD, DHBD and THBD proteins. FIG. 17A depicts the structure and sequence of the 21 bp RNA-DNA hybrid. FIG. 17B through FIG. 17D depict protein titration experiments involving (FIG. 17B) SHBD, (FIG. 17C) DHBD, and (FIG. 17D) THBD. Binding reactions were prepared using purified protein, and a hybrid carrying a ³²P label at the RNA 5′-end, and at fixed concentration of 2, 1, and 0.1 nM for experiments involving the SHBD, DHBD, and THBD, respectively. Two-fold serial dilutions of protein were used, with the lowest and highest concentrations indicated above the images. Reactions were incubated at room temperature for 30 min, then electrophoresed at 150 V in a 0.5×TBE non-denaturing 8% polyacrylamide gel at room temperature. Lane 1 in panels B-D shows the position of the hybrid in the absence of added protein. Arrows with “H” indicate the free hybrid, while arrows with “C” indicate protein-hybrid complexes. The asterisk (*) in FIG. 17B and FIG. 17D indicates a low molecular weight, ³²P-labeled species that copurified with the labeled RNA. Lane ss in FIG. 17D shows the migration of the ³²P-labeled ssRNA, which is also visible beneath the unbound hybrid in lanes 1-10, and is indicated by the arrow “ss”.

FIG. 18A through FIG. 18G depict Surface Plasmon Resonance (SPR) analyses of the interaction of the SHBD, DHBD, and THBD proteins with a RNA-DNA hybrid. FIG. 18A depict the sequence and structure of a 47 nt chimeric oligonucleotide used for the SPR analyses. Intramolecular base-pairing forms a 21 bp RNA-DNA hairpin structure, with a tetrathymidine loop, and a biotin moiety at the 5′-end that tethers the hybrid to the neutravidin-modified sensor chip surface. FIG. 18B through FIG. 18D depict sensorgrams of protein titrations. Above the sensorgram are provided (i) the protein analyzed, (ii) the maximum response as determined analyzing the equilibrium binding data (Rₘₐₓ−exp), and (iii) the predicted maximum response (Rₘₐₓ−theor), assuming a 1:1 binding stoichiometry. The sensorgrams show sets of binding curves obtained by 60 second injections of (FIG. 18B) SHBD protein solutions (0.234, 0.469, 0.938, 1.875, 3.75, 7.5, 15, 30, 60 μM concentrations); (FIG. 18C) DHBD protein solutions (0.977, 1.953, 3.906, 7.813, 15.625, 31.25, 62.5, 125 and 250 nM concentrations); and (FIG. 18D) THBD protein solutions (0.012, 0.049, 0.195, 0.781, 3.125, 12.5 nM concentrations). The time scale starts at the injection point, and triplicates for each experiment are shown. Panels FIG. 18E through FIG. 18G show plots of the equilibrium response as a function of the SHBD (FIG. 18E), or DHBD (FIG. 18F), or THBD (FIG. 18G) protein concentrations. SPR data transformation was performed using the global data analysis tool and a 1:1 binding model.

FIG. 19 depicts equilibrium and kinetic parameters for the interaction of SHBD, DHBD, and THBD proteins with the 21 bp hybrid hairpin, as determined by SPR. The dissociation constants (KD), kinetic parameters (kon, koff) and stoichiometries (protein molecules bound per hybrid) were determined using the KD values determined by an affinity model. The reported values are averages of three separate experiments, with standard deviations. ND, not determinable.

FIG. 20A and FIG. 20B depict determinants of multi-HBD protein binding to an RNA-DNA hybrid. FIG. 20A depicts an illustration of the crystal structure of the human RNase H1 HBD [adapted from PDB ID 3BSU (Nowotny et al., 2008, EMBO J. 27, 1172-1181)]. Three HBDs (beige) can complex with a 12 bp RNA-DNA hybrid (Cyan strand, DNA; Blue strand, RNA). A single HBD directly contacts 5 bp of hybrid, and two consecutive, adjacent HBDs can formally accommodate a 2 bp overlap in binding by a rotational translation along the double helix. Chimera rendering software (Pettersen et al., 2004, J. Comput. Chem. 25, 1605-1612) allowed determination of the distance between the N-terminus and C-terminus of adjacent HBDs (dashed red lines). A fully extended 10aa (3.5 Å/aa) linker would be compatible with supporting multi-HBD protein binding to a hybrid. FIG. 20B depicts a plot illustrating the enhancement of hybrid binding affinity as a function of number of linked HBDs. The affinity was normalized to two linked HBDs, that provided an absolute theoretical binding enhancement of ˜350 fold relative to the HBD.

FIG. 21 depicts the binding dynamics of the multi-HBD protein. Enhancement was calculated as KAapp HBDn+1/KAapp HBDn, where n is the number of HBDs that are linked together to make the protein

### DETAILED DESCRIPTION

The invention described herein, termed epiprinter, is a functionally versatile molecular entity that can detect RNA-DNA hybrids with high sensitivity and selectivity, thus providing the basis for the proposed technology. The epiprinter technology is designed for use in standard laboratory settings as well as in settings for which the rapidity, portability, and cost-effectiveness of the technology are essential features. Epiprinter technology affords a method to detect RNA without requirement for enzyme or a nucleic acid amplification step. The epiprinter, used in conjunction with custom DNA nanostructures and solid-state nanopores, is also designed to provide multiplexed detection of RNA. In this regard, epiprinting provides high throughput, single-molecule detection capability. Epiprinter detection of RNA also is designed to be nondestructive to the RNA sample, allowing further analysis of the RNA, as needed. With simple modification the epiprinter may be used to detect RNA-DNA hybrids in cellulo and in situ, with high sensitivity and accuracy. The epiprinter can also be used for detection of DNA, protein or other molecules, in conjunction with antibodies or aptamers. Therefore, epiprinter technology provides sensitive, accurate, rapid, and convenient detection of biomolecules in virtually any setting, taking advantage of the high specificity achieved by the association of complementary RNA and DNA sequences, the accessibility and functionality of custom-designed DNA nanostructures, a robust and elective chemical reaction, and the high throughput, single-molecule detection capability of solid-state nanopore-based technologies. The specificity, high affinity, and versatile chemical functionality of the epiprinter provides a new and better ways to detect RNA and RNA-DNA hybrids, as well as proteins and other molecular entities, with broad medical, biotechnological, agricultural, and basic research applications.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, an “adaptor” of the present invention means a piece of nucleic acid that is added to a nucleic acid of interest, e.g., the polynucleotide. Two adaptors of the present invention are preferably ligated to the ends of a DNA fragment cross-linked to a polypeptide of interest, with one adaptor on each end of the fragment. Adaptors of the present invention can comprise a primer binding sequence, a random nucleotide sequence, a barcode, or any combination thereof.

An affinity label, as the term us used herein, refers to a moiety that specifically binds another moiety and can be used to isolate or purify the affinity label, and compositions to which it is bound, from a complex mixture. One example of such an affinity label is a member of a specific binding pair (e.g., biotin:avidin, antibody:antigen). The use of affinity labels such as digoxigenin, dinitrophenol or fluorescein, as well as antigenic peptide ‘tags’ such as polyhistidine, FLAG, HA and Myc tags, is envisioned.

“Amplification,” as used herein, refers to any in vitro process for increasing the number of copies of a nucleotide sequence or sequences, i.e., creating an amplification product which may include, by way of example additional target molecules, or target-like molecules or molecules complementary to the target molecule, which molecules are created by virtue of the presence of the target molecule in the sample. These amplification processes include but are not limited to polymerase chain reaction (PCR), multiplex PCR, Rolling Circle PCR, ligase chain reaction (LCR) and the like. An amplification product can be made enzymatically with DNA or RNA polymerases or reverse transcriptases. Nucleic acid amplification results in the incorporation of nucleotides into DNA or RNA. As used herein, one amplification reaction may consist of many rounds of DNA replication. PCR is an example of a suitable method for DNA amplification. For example, one PCR reaction may consist of 2-40 “cycles” of denaturation and replication.

“Amplification products,” “amplified products” “PCR products” or “amplicons” comprise copies of the target sequence and are generated by hybridization and extension of an amplification primer. This term refers to both single stranded and double stranded amplification primer extension products which contain a copy of the original target sequence, including intermediates of the amplification reaction.

“Appropriate hybridization conditions” as used herein may mean conditions under which a first nucleic acid sequence (e.g., primer, etc.) will hybridize to a second nucleic acid sequence (e.g., target, etc.), such as, for example, in a complex mixture of nucleic acids. Appropriate hybridization conditions are sequence-dependent and will be different in different circumstances. In one embodiment, appropriate hybridization conditions may be selective or specific wherein a condition is selected to be about 5-10° C. lower than the thermal melting point (Tm) for the specific sequence at a defined ionic strength and pH. In one embodiment, an appropriate hybridization condition encompasses hybridization that occurs over a range of temperatures from more to less stringent. In one embodiment, a hybridization range may encompass hybridization that occurs from 98° C. to 50° C. According to the invention, such a hybridization range may be used to allow hybridization of the primers of the invention to target sequences with reduced specificity, for the purposes of amplifying a broad range of nucleic acid molecules with a single set of primers.

As used herein, “binding” means an association interaction between two molecules, via covalent or non-covalent interactions including, but not limited to, hydrogen bonding, hydrophobic interactions, van der Waals interactions, and electrostatic interactions. Binding may be sequence specific or non-sequence specific. Non-sequence specific binding may occur when, for example, a polypeptide of interest (i.e. a histone) binds to a polynucleotide of any sequence. Specific binding may occur when, for example, a polypeptide of interest (i.e. a transcription factor) binds predominantly to a highly restricted sequence of nucleotides.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

As used herein, the term “derived” or “directed to” with respect to a nucleotide molecule means that the molecule has complementary sequence identity to a particular molecule of interest.

As used herein, “dNTPs” refers to a mixture of different deoxyribonucleoside triphosphates: deoxyadenosine triphosphate (dATP), deoxycytidine triphosphate (dCTP), deoxyguanosine triphosphate (dGTP) and deoxythymidine triphosphate (dTTP).

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

“Fragment” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. When comparing DNA and RNA, thymine (T) and uracil (U) may be considered equivalent. Identity may be performed manually or by using a computer sequence algorithm such as BLAST or BLAST 2.0.

“Nucleic acid” or “oligonucleotide” or “polynucleotide” or “nucleic acid fragment” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence. Thus, a nucleic acid also encompasses a probe that hybridizes under appropriate hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine.

Modified nucleotides are known in the art and include, by example and not by way of limitation, alkylated purines and/or pyrimidines; acylated purines and/or pyrimidines; or other heterocycles. These classes of pyrimidines and purines are known in the art and include, pseudoisocytosine; N4, N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2-methylthio-N6-isopentenyl-adenine; uracil-5-oxyacetic acid methyl ester; pseudouracil; 2-thiocytosine; 5-methyl-2 thiouracil, 2-thiouracil; 4-thiouracil; 5-methyluracil; N-uracil-5-oxyacetic acid methyl ester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpseudouracil; 1-methylguanine; 1-methylcytosine. Backbone modifications are similarly known in the art, and include, chemical modifications to the phosphate linkage (e.g., phosphorodiamidate, phosphorothioate (PS), N3′ phosphoramidate (NP), boranophosphate, 2′,5′ phosphodiester, amide-linked, phosphonoacetate (PACE), morpholino, peptide nucleic acid (PNA) and inverted linkages (5′-5′ and 3′-3′ linkages)) and sugar modifications (e.g., 2′-O-Me, UNA, LNA).

A “mutation” or “variation” as used herein, refers to a change in nucleic acid or polypeptide sequence relative to a parental or reference sequence, and includes translocations, deletions, insertions, and substitutions/point mutations. A “mutant” or “variant” as used herein, refers to either a nucleic acid or protein comprising a mutation.

As used herein, the term “nanostructure” is defined to mean any structure having a distinct shape formed from a plurality of elements. For example, the shape may include linear forms, circular forms, two-dimensional patterns or three-dimensional structures. Preferably, at least one dimension of the structure is on the nanoscale, i.e. in the range between 0.1 and 100 nm. For example, two-dimensional patterns may have a thickness on the nanoscale. Nanotubes preferably have two dimensions on the nanoscale, i.e. the diameter of the tube is between 0.1 and 100 nm while the length could be much greater.

The oligonucleotides described herein may be synthesized using standard solid or solution phase synthesis techniques which are known in the art. In certain embodiments, the oligonucleotides are synthesized using solid-phase phosphoramidite chemistry (U.S. Pat. No. 6,773,885) with automated synthesizers. Chemical synthesis of nucleic acids allows for the production of various forms of the nucleic acids with modified linkages, chimeric compositions, and nonstandard bases or modifying groups attached in chosen places through the nucleic acid's entire length.

Certain embodiments of the invention encompass isolated or substantially purified nucleic acid compositions. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule is substantially free of other cellular material or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

“Operably-linked” refers to the association of two chemical moieties so that the function of one is affected by the other, e.g., an arrangement of elements wherein the components so described are configured so as to perform their usual function.

“Primer” as used herein refers to a single-stranded oligonucleotide or a single-stranded polynucleotide that is extended on its 3′ end by covalent addition of nucleotide monomers during amplification. Nucleic acid amplification often is based on nucleic acid synthesis by a nucleic acid polymerase. Many such polymerases require the presence of a primer that can be extended to initiate such nucleic acid synthesis.

As used herein, “purifying” the polynucleotides of the present invention refers to a process well known to those of skill in the art in which polynucleotides are substantially separated from other components in a sample, including, but not limited to, polypeptides of interest.

As used herein, “sample” or “test sample,” may refer to any source used to obtain nucleic acids for examination using the compositions and methods of the invention. A test sample is typically anything suspected of containing a target sequence. Test samples can be prepared using methodologies well known in the art such as by obtaining a specimen from an individual and, if necessary, disrupting any cells contained thereby to release genomic nucleic acids. These test samples include biological samples which can be tested by the methods of the present invention described herein and include human and animal cells, tissues and body fluids such as whole blood, serum, plasma, cerebrospinal fluid, sputum, bronchial washing, bronchial aspirates, urine, lymph fluids and various external secretions of the respiratory, intestinal and genitourinary tracts, tears, saliva, milk, white blood cells, myelomas, buccal cells, cervicovaginal cells, epithelial cells from urine, fetal cells, or any cells present in tissue obtained by biopsy and the like; biological fluids such as cell culture supernatants; tissue specimens which may be fixed; and cell specimens which may be fixed.

“Substantially complementary” as used herein may mean that a first sequence is at least 95%, 96%, 97%, 98% or 99% identical to the complement of a second sequence over a region of about 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under appropriate hybridization conditions.

“Substantially identical” as used herein may mean that a first and second sequence are at least 95%, 96%, 97%, 98%, or 99% over a region of about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The epiprinter technology described herein provides compositions and methods for sensitive, accurate, rapid, and convenient detection of molecules and biomolecules in virtually any setting, taking advantage of the high specificity achieved by the association of complementary RNA and DNA sequences, the accessibility and functionality of custom-designed DNA nanostructures, a robust and elective chemical reaction, and the high throughput, single-molecule detection capability of solid-state nanopore-based technologies.

### DNA:RNA Hybrid Binding Molecule

The invention is based, in part on the development of an RNA:DNA hybrid binding molecule comprising at least one hybrid binding domain (HBD). Therefore, in some embodiments, the invention provides an RNA:DNA hybrid binding molecule comprising at least one RNA:DNA hybrid binding domain (HBD) or a nucleic acid molecule encoding the same. In one embodiment, the RNA:DNA hybrid binding molecule comprises two RNA:DNA hybrid binding domains (DHBD). In one embodiment, the RNA:DNA hybrid binding molecule comprises three RNA:DNA hybrid binding domains (THBD).

In certain embodiments, the RNA:DNA hybrid binding molecule, comprising a single HBD, comprises at least about 75% sequence identity to SEQ ID NO:2. In certain embodiments, the HBD epiprinter comprises at least about 95%, 96%, 97%, 98%, 99% or 100% sequence identity to SEQ ID NO:2. In certain embodiments, the nucleotide sequence encoding the RNA:DNA hybrid binding molecule comprises at least about 75% sequence identity to SEQ ID NO:1. In certain embodiments, the nucleotide sequence encoding the HBD comprises at least about 95%, 96%, 97%, 98%, 99% or 100% sequence identity to SEQ ID NO:1.

In certain embodiments, the RNA:DNA hybrid binding molecule comprising at least two HBDs (DHBD) comprises at least about 75% sequence identity to SEQ ID NO:8 or SEQ ID NO:19. In certain embodiments, the DHBD hybrid binding molecule comprises at least about 95%, 96%, 97%, 98%, 99% or 100% sequence identity to SEQ ID NO:8 or SEQ ID NO:19. In certain embodiments, the nucleotide sequence encoding the DHBD hybrid binding molecule comprises at least about 75% sequence identity to SEQ ID NO:7 or SEQ ID NO:18. In certain embodiments, the nucleotide sequence encoding the DHBD hybrid binding molecule comprises at least about 95%, 96%, 97%, 98%, 99% or 100% sequence identity to SEQ ID NO:7 or SEQ ID NO:18.

In certain embodiments, the RNA:DNA hybrid binding molecule comprising at least three HBDs (TBHD) comprises at least about 75% sequence identity to SEQ ID NO:14 or SEQ ID NO:16. In certain embodiments, the THBD hybrid binding molecule comprises at least about 95%, 96%, 97%, 98%, 99% or 100% sequence identity to SEQ ID NO:14 or SEQ ID NO:16. In certain embodiments, the nucleotide sequence encoding the THBD hybrid binding molecule comprises at least about 75% sequence identity to SEQ ID NO:13 or SEQ ID NO:15. In certain embodiments, the nucleotide sequence encoding the THBD hybrid binding molecule comprises at least about 95%, 96%, 97%, 98%, 99% or 100% sequence identity to SEQ ID NO:13 or SEQ ID NO:15.

In certain embodiments, a nucleotide sequence encoding the HBD, DHBD or THBD hybrid binding molecule comprises at least 168 nucleotides of SEQ ID NO:1 in the sense or anti-sense direction. Exemplary nucleotide sequences comprising at least 168 nucleotides of SEQ ID NO:1 include, but are not limited to: SEQ ID NO:5 and SEQ ID NO:6, which together combine to encode a DHBD hybrid binding molecule, following BsaI digestion and ligation; SEQ ID NO:17 and SEQ ID NO:6, which together combine to encode a DHBD hybrid binding molecule, following BsaI digestion and ligation; SEQ ID NO:9, SEQ ID NO:11 and SEQ ID NO:12, which together combine to encode a THBD hybrid binding molecule, following BsaI digestion and ligation; and SEQ ID NO:10, SEQ ID NO:11 and SEQ ID NO:12, which together combine to encode a THBD-1C hybrid binding molecule, following BsaI digestion and ligation.

In one embodiment, the invention relates to compositions comprising a nucleotide sequence encoding at least one HBD, DHBD, THBD, or a fragment or variant thereof. In various embodiments, the invention includes an isolated nucleic acid encoding at least one HBD, DHBD, THBD, or a fragment or variant thereof, operably linked to a nucleic acid comprising a promoter/regulatory sequence such that the nucleic acid is preferably capable of directing expression of the at least one HBD binding molecule encoded by the nucleic acid. Thus, the invention encompasses expression vectors for expression of a HBD binding molecule of the invention, and methods for the introduction of exogenous DNA into cells with concomitant expression of the exogenous DNA in the cells.

### Epiprinter

In some embodiments, the invention provides an epiprinter molecule comprising at least one RNA-DNA hybrid binding domain (HBD) and at least one reactive moiety. In one embodiment, the epitape molecule comprises a reactive site and the epiprinter molecule comprises a reactive moiety which, under mild conditions, permits conjugation of the epiprinter to the epitape.

In some embodiments, the invention provides an epiprinter molecule comprising at least one RNA:DNA hybrid binding domain (HBD) and at least one moiety capable of participating in a “click chemistry” or cycloaddition reaction. In one embodiment, the epiprinter molecule comprises two RNA:DNA hybrid binding domains (DHBD) and at least one reactive moiety. In one embodiment, the epiprinter molecule comprises two RNA:DNA hybrid binding domains and two reactive moieties (DHBD-2C). In one embodiment, the epiprinter molecule comprises three RNA:DNA hybrid binding domains (THBD) and at least one reactive moiety. In one embodiment, the epiprinter molecule comprises three RNA:DNA hybrid binding domains and a single cysteine residue (THBD-1C).

In certain embodiments, the HBD epiprinter comprises at least about 75% sequence identity to SEQ ID NO:2. In certain embodiments, the HBD epiprinter comprises at least about 95%, 96%, 97%, 98%, 99% or 100% sequence identity to SEQ ID NO:2. In certain embodiments, the nucleotide sequence encoding the HBD epiprinter comprises at least about 75% sequence identity to SEQ ID NO:1. In certain embodiments, the nucleotide sequence encoding the HBD comprises at least about 95%, 96%, 97%, 98%, 99% or 100% sequence identity to SEQ ID NO:1.

In certain embodiments, the DHBD epiprinter comprises at least about 75% sequence identity to SEQ ID NO:8. In certain embodiments, the DHBD epiprinter comprises at least about 95%, 96%, 97%, 98%, 99% or 100% sequence identity to SEQ ID NO:8. In certain embodiments, the nucleotide sequence encoding the DHBD epiprinter comprises at least about 75% sequence identity to SEQ ID NO:7. In certain embodiments, the nucleotide sequence encoding the DHBD comprises at least about 95%, 96%, 97%, 98%, 99% or 100% sequence identity to SEQ ID NO:7.

In certain embodiments, the DHBD-2C epiprinter comprises at least about 75% sequence identity to SEQ ID NO:19. In certain embodiments, the DHBD-2C epiprinter comprises at least about 95%, 96%, 97%, 98%, 99% or 100% sequence identity to SEQ ID NO:19. In certain embodiments, the nucleotide sequence encoding the DHBD-2C epiprinter comprises at least about 75% sequence identity to SEQ ID NO:18. In certain embodiments, the nucleotide sequence encoding the DHBD-2C comprises at least about 95%, 96%, 97%, 98%, 99% or 100% sequence identity to SEQ ID NO:18.

In certain embodiments, the THBD epiprinter comprises at least about 75% sequence identity to SEQ ID NO:14. In certain embodiments, the THBD epiprinter comprises at least about 95%, 96%, 97%, 98%, 99% or 100% sequence identity to SEQ ID NO:14. In certain embodiments, the nucleotide sequence encoding the THBD epiprinter comprises at least about 75% sequence identity to SEQ ID NO:13. In certain embodiments, the nucleotide sequence encoding the THBD comprises at least about 95%, 96%, 97%, 98%, 99% or 100% sequence identity to SEQ ID NO:13.

In certain embodiments, the THBD-1C epiprinter comprises at least about 75% sequence identity to SEQ ID NO:16. In certain embodiments, the THBD-1C epiprinter comprises at least about 95%, 96%, 97%, 98%, 99% or 100% sequence identity to SEQ ID NO:16. In certain embodiments, the nucleotide sequence encoding the THBD-1C epiprinter comprises at least about 75% sequence identity to SEQ ID NO:15. In certain embodiments, the nucleotide sequence encoding the THBD-1C comprises at least about 95%, 96%, 97%, 98%, 99% or 100% sequence identity to SEQ ID NO:15.

In certain embodiments, a nucleotide sequence encoding the HBD, DHBD or THBD epiprinter comprises at least 168 nucleotides of SEQ ID NO:1 in the sense or anti-sense direction. Exemplary nucleotide sequences comprising at least 168 nucleotides of SEQ ID NO:1 include, but are not limited to: SEQ ID NO:5 and SEQ ID NO:6, which together combine to encode a DHBD epiprinter; SEQ ID NO:17 and SEQ ID NO:6, which together combine to encode a DHBD-2C epiprinter; SEQ ID NO:9, SEQ ID NO:11 and SEQ ID NO:12, which together combine to encode a THBD epiprinter; and SEQ ID NO:10, SEQ ID NO:11 and SEQ ID NO:12, which together combine to encode a THBD-1C epiprinter.

In a non-limiting example, the reactive moiety is an azide, and the reactive site of the epiprinter molecule comprises at least one alkyne. Under mild conditions, the alkyne and azide undergo a [3+2] cyclization reaction to produce a triazole, thereby conjugating the epiprinter to the epitape via the triazole moiety. It should be understood that the reactive moiety and the reactive site are interchangeable, permitting an equivalent conjugation reaction wherein the functionality between the reactive moiety and the reactive site on the biomolecule have been switched. In another non-limiting example, the reactive site comprises an alkyl or aryl bromide or a maleimide. These reactive sites can react with a sulfur group or an amine on the epiprinter, in order to conjugate the epiprinter to the epitape molecule. In addition, alkyl or aryl bromides and maleimides form covalent bonds with cysteine residues in proteins under mild conditions. Other non-limiting examples of reactive moieties include amines and carbonyl groups such as aldehydes or ketones. Aldehydes and ketones may undergo reaction with amines, thereby conjugating a molecule comprising a carbonyl group to an amine on the epiprinter molecule.

In one embodiment, the epiprinter molecule comprises an azide moiety. For example, in one embodiment, the epiprinter molecule comprises a non-canonical amino acid comprising an azide group. Exemplary non-canonical amino acids comprising an azide group include, but are not limited to, p-azido-L-phenylalanine and azidohomoalanine (AHA).

In some embodiments, the two or three HBD of the epiprinter molecule are connected by linkers. In some embodiments, the linker comprises at least one reactive moiety. In various embodiments, the linker comprises at least 3, 4, 5, 6, 7, 8, 9, 10, 11, 12, 13, 14, 15, 16, 17, 18, 19, 20 or more than 20 amino acid residues. In some embodiments the linker comprises a sequence of CIC, wherein the cysteine (C) residues of the linker comprises a reactive moiety. In some embodiments, the cysteine residues of the linker comprises an alkyl azide that can participate in a cycloaddition reaction. In some embodiments the linker comprises a sequence of SIS, wherein the cysteine (C) residues of the linker are substituted for serine (S) residues which do not participate in a cycloaddition reaction. In some embodiments, p-azido-L-phenylalanine is incorporated into the DHBD or THBD linker and can participate in a cycloaddition reaction.

In one embodiment, the reactive moiety of the epiprinter molecule participates in a click chemistry reaction with the epitape molecule. Click chemistry reaction takes place between two components: azide and alkyne (a terminal acetylene). For example, in one embodiment, the click chemistry reaction is a ring-strain promoted alkyne-azide cycloaddition reaction (SPAAC reaction) (Shelbourne et al. 2011, Chembiochem, 12: 1912-1921), or a copper-(Cu[I])catalyzed alkyne-azide cycloaddition reaction (CuAAC reaction) (El-Sagheer et al. 2012, Acc Chem Res, 45(8): 1258-1267). Another embodiment uses a Diels-Alder reaction in which diene carrying oligonucleotides undergoes cycloaddition with maleimide-terminated fluorescence dyes (Borsenberger et al., 2009, Nucleic Acids Res, 37(5): 1477-1485).

In some embodiments, the epiprinter molecule is labeled. The term “label” when used herein refers to a detectable compound or composition that is conjugated directly or indirectly to a molecule to generate a “labeled” molecule. The label may be detectable by itself (e.g. radioisotope labels or fluorescent labels) or, in the case of an enzymatic label, may catalyze chemical alteration of a substrate compound or composition that is detectable (e.g., avidin-biotin). In some embodiments, the epiprinter is covalently linked to a detectable label through maleimide-sulfhydryl cross-linking of a maleimide-containing detectable label to a sulfhydryl group of the epiprinter. In one embodiment, the epiprinter comprises a single sulfhydryl group, allowing for site-specific labeling of the epiprinter. For example, in one embodiment, the epiprinter comprises a sequence as set forth in SEQ ID NO:16, comprising a single cysteine residue.

### DNA-Nanostructures

DNA-nanostructures are nanoscale structures made of DNA, wherein the DNA acts both as a structural and functional element. DNA-nanostructures can serve as a scaffold for the formation of other structures. DNA-nanostructures may be prepared by methods known in the art using oligodeoxynucleotides. For example, such nanostructures may be assembled based on the concept of base-pairing, and while no specific sequence is required, the sequences of each oligonucleotide must be partially complementary to certain other oligonucleotides to enable hybridization of all strands.

A nucleic acid of the invention also includes artificial genetic polymers, commonly referred to as XNAs or ‘xeno-nucleic acids’ where the backbone structure contains a sugar other than ribose or deoxyribose. While some of these molecules can be considered natural derivatives of RNA, like arabino nucleic acid (ANA), threose nucleic acid (TNA), and glycerol nucleic acid (GNA), others are completely unnatural, like locked nucleic acid (LNA), cyclohexene nucleic acid (CeNA), and hexitol nucleic acid (HNA).

Therefore in one embodiment, the invention provides artificial or synthetic nucleic acid molecules which incorporate one or more natural or modified nucleosides. The length of the nucleic acids may vary. The nucleic acids may be modified, e.g. may comprise one or more modified nucleobases or modified sugar moieties (e.g., comprising methoxy groups). The backbone of the nucleic acid may comprise one or more peptide bonds as in peptide nucleic acid (PNA). The nucleic acid may comprise a base analog such as non-purine or non-pyrimidine analog or nucleotide analog. It may also comprise additional attachments such as proteins, peptides and/or or amino acids.

A nucleic acid oligonucleotide of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 1419), phosphorothioate (Mag et al. (1991) Nucleic Acids Res. 19:1437; and U.S. Pat. No. 5,644,048), phosphorodithioate (Briu et al. (1989) J. Am. Chem. Soc. 111:2321, O-methylphosphoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The length of each oligonucleotide or DNA strand is variable and depends on, for example, the type of nanostructure. In certain embodiments, the oligonucleotide or DNA strand is about 15 nucleotides in length to about 500 nucleotides in length, about 15 to about 200 nucleotides in length, or about 15 to about 100 nucleotides in length.

For use in the present invention, the nucleic acids can be synthesized de novo using any of a number of procedures well known in the art. Nucleic acids may be isolated from natural sources or purchased from commercial sources. In certain exemplary embodiments, nucleic acids or nucleic acid-binding molecules may be prepared using one or more of the phosphoramidite linkers and/or synthesized by ligation methods known to those of skill in the art. For example, the cyanoethyl phosphoramidite method (Beaucage, S. L., and Caruthers, M. H., Tet. Let. 22:1859, 1981); nucleoside H-phosphonate method (Garegg et al., Tet. Let. 27:4051-4054, 1986; Froehler et al., Nucl. Acid. Res. 14:5399-5407, 1986; Garegg et al., Tet. Let. 27:4055-4058, 1986, Gaffney et al., Tet. Let. 29:2619-2622, 1988), or by any other chemical method using either a commercial automated oligonucleotide synthesizer or high-throughput, high-density array methods known in the art. Pre-synthesized oligonucleotides may also be obtained commercially from a variety of vendors.

In certain exemplary embodiments, nucleic acids may be prepared using a variety of micro-array technologies known in the art. Pre-synthesized nucleic acids or nucleic acid-binding molecules may be attached to a support or synthesized in situ using light-directed methods, flow channel and spotting methods, ink-jet methods, pin-based methods and bead-based methods known in the art.

Nucleic acid origami structures, also referred to as DNA origami structures or DNA origami, are two- or three-dimensional assemblages of specific shapes formed from nucleic acids. The term “origami” infers that one or more strands or building blocks of DNA (called scaffold strands) may be folded or otherwise positioned into a desired structure or shape. The desired structure or shape are then stabilized by one or more additional strands or building blocks of DNA (called staples). Methods of DNA origami are described for example by Rothemund, 2006, Nature, 440:297-302; Douglas et al., 2009, Nature, 459: 414-418; and Seeman, 2010, Biochem. 79:65-87, all of which are incorporated herein by reference in their entirety.

A nucleic acid origami structure designed for use in the systems and methods of the invention can be constructed using single-stranded nucleic acid sequences which self-assemble into structures of the desired shape, size, and functionality. Such approaches include the programmed self-assembly of designed strands of nucleic acids to create a wide range of structures with the desired shapes (Wei et al., 2012, Nature, 485:623-627; herein incorporated by reference in its entirety).

A DNA nanostructure for use in the systems and methods of the invention may be of any arbitrary shape as desired, including, but not limited to DNA origami bundles, a rectangular DNA origami nanostructure, a triangular DNA origami nanostructure, a tubular DNA origami nanostructure, a tetrahedral DNA origami nanostructure, a pentahedral DNA origami nanostructure, a hexahedral DNA origami nanostructure, a septahedral DNA origami nanostructure, an octahedral DNA origami nanostructure, a nonahedral DNA origami nanostructure, a decahedral DNA origami nanostructure, a hendecahedral DNA origami nanostructure, and a dodecahedral DNA origami nanostructure.

### Epitape

In some embodiments, the invention provides DNA nanostructures referred to herein as epitapes or epitape molecules. In some embodiments, the epitape molecules of the invention are DNA origami molecules that self-assemble from a long single stranded polynucleotide scaffold, and oligonucleotide “staples.”

In general, the basic technique for creating an epitape of the invention involves folding a long single stranded polynucleotide into a desired shape or structure using a set of short “staple” nucleic acid strands as “glue” to fix the polynucleotide strand into a particular, stable pattern or shape. The choice of staple strands determines the pattern. In one embodiment, the epitape is comprised of a long single stranded polynucleotide, comprised of the circular ssDNA of the M13 phage, and a mixture of oligonucleotide staples, that self-assembles into a stiff linear bundle comprising at least two double helix strands.

### Probe Regions

In some embodiments, the epitape molecules contain one or more probe regions, or probe sites, that allow for specific binding to an RNA molecule of interest. In one embodiment, the epitape molecule contains at least 1, 2, 3, 4, 5, 6, 7, 8, 9, 10, or more than 10 probe sites. In one embodiment, the epitape molecule contains at least two probe sites, wherein each probe site has a DNA sequence complementary to an RNA target sequence of interest. In one embodiment, the at least two probe sites are specific for binding to the same RNA target sequence. In one embodiment, the at least two probe sites are specific for binding to the two or more different RNA target sequence. In one embodiment, at least one probe site is specific for binding to a control or reference sequence, and at least one probe site is specific for binding to a target of interest.

In one embodiment, the epitape molecule contains at least two probe sites, wherein each probe site comprise at least two probe strands, wherein each probe strand comprises a target binding region comprising a sequence complementary to a target sequence of interest and a staple region which functions to hybridize to and stabilize the epitape molecule. In one embodiment, the at least two probe strands that together make a probe sites are specific for binding to different sequences of the same target sequence.

In one embodiment, the probe site comprises at least 20, 25, 30, 35, 40, 45, 50, 55, 60 or more than 60 nucleotides complementary to a nucleic acid target sequence of interest. In some embodiments, each probe site comprises 35 to 50 nucleotides complementary to a nucleic acid target sequence of interest.

In one embodiment, each probe strand of a probe site comprises at least 10, 11, 12, 13, 14, 15, 16, 17, 18, 19, 20, 21, 22, 23, 24, 25, 26, 27, 28, 29, 30, or more than 30 nucleotides complementary to a nucleic acid target sequence of interest. In some embodiments, each probe strand comprises 17 to 25 nucleotides complementary to a nucleic acid target sequence of interest.

In one embodiment, each probe strand of a probe site specific for binding to a target of interest comprises at least one internal alkynyl group, such that the probe site comprises two terminal alkynyl groups. In such an embodiment, the two terminal alkynyl groups participate in a click chemistry reaction with the epiprinter, following binding of the RNA target to the DNA probe which forms an RNA:DNA hybrid that is recognized by the HBD of the epiprinter.

In one embodiment, each probe strand of a probe site specific for binding to a target of interest comprises at least one Bicyclo[6.1.0] nonyne (BCN) group, or a structural analog thereof, attached to specific probe site dU bases. In such an embodiment, the BCN groups, or the BCN structural analogs participate in a Cu-independent Strain-Promoted Azide-Alkyne Cycloaddition (SPAAC) reaction with the epiprinter following binding of the RNA target to the DNA probe which forms an RNA:DNA hybrid that is recognized by the HBD of the epiprinter.

In one embodiment, each probe strand of a probe site specific for binding to a target of interest comprises at least one alkyl azide group on an internal dU base. In such an embodiment, the alkyl azide group(s) can participate in a Cu-independent Strain-Promoted Azide-Alkyne Cycloaddition (SPAAC) reaction with BCN groups, or the BCN structural analogs on the epiprinter following binding of the RNA target to the DNA probe which forms an RNA:DNA hybrid that is recognized by the HBD of the epiprinter.

### Barcodes

In some embodiments, the epitape molecules contain unique sequences (barcodes) that allow identification of individual epitape molecules used in multiplexed assays. In some embodiments, barcoding is generated through a unique pattern of nucleic acid structures in the barcode region of an epitape.

In one embodiment, the barcode element is comprised of specific combinations of multiple individual signaling units. In one embodiment, an individual signaling unit is a localized DNA structure in the proto-epitape that causes a localized steric enlargement of the epitape, detectable during nanopore translocation in a unique, consistent manner. Exemplary barcodes include, but are not limited to, bulges or enlargements in the epitape structure, DNA secondary structures that protrude from the epitape, and molecular entities that are covalently attached to the epitape, such as a small protein, or a molecular polymer (e.g. polyethylene glycol). Exemplary DNA secondary structures that can be designed to protrude from the epitape and therefore used to generate a barcode include, but are not limited to, a DNA hairpin and DNA dumbbell structures. In one embodiment, a specific staple in the epitape is lengthened to include a sequence that forms a DNA secondary structure that then protrudes from the epitape. The resulting protrusion creates a transient downward spike in the current signal during translocation.

In one embodiment, a barcode signaling unit is created by covalently attaching a molecular entity to the epitape. In one embodiment, specific staples are commercially modified at specific nucleotides with a broad range of chemical groups, including those that allow click-chemistry, or sulfhydryl-maleimide reactions. For example, in one embodiment, a small protein, or a molecular polymer (e.g. polyethylene glycol), modified with the complementary click-chemistry group, attaches to specific modified staples of the epitape, and the resultant structure creates a transient downward spike in the current signal during translocation.

In one embodiment, the epitape molecule comprising a barcode region containing several signaling units in a specific array on the epitape. In some embodiments, the epitape molecules of the invention comprise a barcode region comprising at least 1, at least 2, at least 3, at least 4, at least 5 or more than 5 barcode regions, wherein each barcode region comprises N signaling units, with N being 0, 1, 2, 3, 4, 5 or more than 5.

In one embodiment, the structure of the signaling unit allows the barcode to provide an unambiguous, easily interpretable output signal. Unique barcodes on the epitape molecules enables implementation of multiplexing in target detection and also allows determination of the directionality of the epitape during translocation.

For example, in one embodiment, a barcode may consist of a unique pattern of nucleic acid (DNA) hairpin structures within a specific region of the epitape, which would provide a digital readout. In one embodiment, the presence of at least one nucleic acid hairpin structure at a given position in an epitape barcode region would provide a binary “1”, and the absence of at least one nucleic acid hairpin structure at a given position in an epitape barcode region would be indicated as a binary “0.”

### Nanopore Systems

Nanopores offer a unique capability of sensing molecules in a label-free manner. In a typical nanopore measurement, an insulating membrane separates two chambers containing an electrolyte solution, and analyte molecules in the solution are electrophoretically driven across the barrier via a nanometer-scale aperture (pore) contained in the membrane. A characteristic transient drop in the ionic conductance of the pore is observed for each passing molecule, and the specific characteristics of the current drop is used to determine the identity and features of the passing molecule.

In some embodiments, transit of an epiprinter/epitape complex through the pore impedes passage of ions through the pore, thus impeding the electrical current.

In one aspect, the invention is a system for analyzing an epiprinter/epitape complex, the system including: a first reservoir containing an electrically conductive aqueous solution; an electrode disposed within the first reservoir in electrical contact with the electrically conductive aqueous solution (e.g., a LiCl salt solution); a second reservoir containing an electrically conductive aqueous solution; another electrode disposed within the second reservoir and in electrical contact with the electrically conductive aqueous solution; and a membrane separating the two reservoirs, the membrane having a pore of diameter sufficient to allow passage of the epiprinter/epitape complex.

In some embodiments, the pore has a diameter of about 0.3 nm, about 0.4 nm, 0.5 nm, about 0.6 nm, about 0.7 nm, about 0.8 nm, about 0.9 nm, about 1 nm, about 1.5 nm, about 2 nm, about 3 nm, about 4 nm, about 5 nm, about 6 nm, about 7 nm, about 7.5 nm, about 8 nm, about 10 nm, about 12 nm, about 15 nm, about 20 nm, about 25 nm, about 30 nm, about 35 nm, about 40 nm, about 45 nm, about 50 nm, about 60 nm, about 70 nm, about 80 nm, about 90 nm, about 100 nm, about 125 nm, about 150 nm, about 175 nm, about 200 nm, about 250 nm, about 300 nm, about 350 nm, about 400 nm, about 450 nm, or about 500 nm. In some embodiments, the pore has a diameter from about 0.5 to about 500 nm, from about 10 to about 500 nm, from about 25 to about 500 nm, from about 50 to about 500 nm, from about 100 to about 500 nm, from about 0.5 to about 200 nm, from about 0.5 to about 101 nm, from about 0.5 to about 50 nm, from about 0.3 to about 50 nm, from about 1 to about 50 nm, from about 1 to about 30 nm, or from about 0.5 to about 25 nm.

In some embodiments, the pore has a longitudinal length of about 0.2 nm, 0.3 nm, 0.4 nm, 0.5 nm, about 0.6 nm, about 0.7 nm, about 0.8 nm, about 0.9 nm, about 1 nm, about 1.5 nm, about 2 nm, about 2.7 nm, about 3 nm, about 4 nm, about 5 nm, about 6 nm, about 7 nm, about 7.5 nm, about 8 nm, about 10 nm, about 12 nm, about 15 nm, about 20 nm, about 25 nm, about 30 nm, about 35 nm, about 40 nm, about 45 nm, or about 50 nm. In some embodiments, the pore has a longitudinal length of from about 0.5 to about 50 nm, from about 1 to about 50 nm, from about 2.5 to about 50 nm, from about 5 to about 50 nm, from about 10 to about 50 nm, from about 0.5 to about 20 nm, from about 0.5 to about 10 nm, from about 0.5 to about 5 nm, from about 1 to about 5 nm, from about 1 to about 3 nm. of from about 0.3 to about 2.5 nm.

The membrane may be made of any ion-insulating material. In some embodiments, the membrane is made of solid-state material. As used herein, “solid-state” refers to any material that exists as a solid at ambient temperatures. For example, in some embodiments, the membrane may be made of silicon, silicon nitride, silicon dioxide, mica, hafnium oxide, graphene, molybdenum disulfide, or polyimide. Alternatively, the membrane may be made of semi-liquid or liquid crystalline materials, e.g., a lipid bilayer.

In some embodiments, the membrane contains a plurality of pores. For example, the membrane may have at least 5, at least 10, at least 20, at least 50, at least 100, at least 200, at least 500, at least 1000, at least 2000, at least 5000, at least 10,000, at least 20,000, at least 50,000 or at least 100,000 pores.

In some embodiments, the system has multiple membranes, and each membrane has multiple pores. For example, the system may have at least 5, at least 10, at least 20, at least 50, at least 100, at least 200, at least 500, at least 1000, at least 2000, at least 5000, at least 10,000, at least 20,000, at least 50,000 or at least 100,000 pores.

The membrane may be provided as part of a chip that includes a supporting structure, and optionally, an insulating layer. Because the membrane is thin and thus susceptible to being damaged during handling, the supporting structure provides structural strength and rigidity to preserve the integrity of the membrane. The supporting structure may be made of any material and of any thickness suitable for this purpose. For example, the supporting structure may be made of silicon, glass, quartz, sapphire, or mica. An insulating layer may be necessary to electrically insulate the membrane from the supporting structure, so the insulating layer may be made of any material suitable and of any thickness for this purpose. For example, the insulating layer may be made of Si0₂ Hf0₂ or AI₂O₃. To provide access to the membrane, the supporting structure and insulating layer, if present, contain one or more windows in which the membrane is not in contact with the supporting structure or insulating layer. The window, as viewed from a sight line perpendicular to the membrane, may be of any shape. For example, the window may be rectangular, square, or circular. The minimum length across the window must be sufficient to allow access to the region of the membrane containing the pore.

The nanopore devices also generally comprise a means for applying an electric field between the reservoirs. In some embodiments, the electric field applying means is typically capable of generating a voltage of at least about 10 mV, about 50 mV, or at least about 100 mV. In one embodiment, the electric field generating means is made up of silver chloride electrodes positioned in the reservoirs that are connected to a voltage source.

The device typically further comprises a means for monitoring the current flow through the channel and processing the observed current flow to produce a usable output. Generally, such monitoring means includes a very low noise amplifier and current injector, and an analog-to-digital (A/D) converter. The device may further comprise other elements of the output generating system, including data acquisition software, an electronic storage medium, etc.

### Methods for Detection of RNA:DNA Hybrid Molecules

In some embodiments, the RNA:DNA hybrid binding molecules of the invention, and the nucleic acid molecules encoding the same can be used to detect the presence of an RNA:DNA hybrid molecule in a sample.

In some embodiments, the RNA:DNA hybrid molecule is a biomarker of a disease or disorder. Biomarkers that can be detected using the systems and methods of the invention include, but are not limited to, viral nucleic acid molecules, bacterial nucleic acid molecules, a microRNA molecule, an mRNA molecule, an alternatively spliced mRNA molecule, a nucleic acid molecule harboring a disease-associated mutation, and other biomarkers associated with a disease or disorder.

In one embodiment, the invention provides methods of detecting an RNA:DNA hybrid molecule of interest. In one embodiment, the method comprises the steps of a) contacting a sample comprising an RNA:DNA hybrid molecule of interest with an RNA:DNA binding molecule of the invention and b) detecting the binding of the RNA:DNA binding molecule to the RNA:DNA hybrid molecule of interest.

### Methods for Detection of Molecules and Biomolecules

The system of the invention can be used to detect the presence of a molecule or biomolecule of interest in a sample. In some embodiments, biomolecule may be a polynucleotide, e.g., a DNA or RNA molecule and the system can be used to directly detect the presence of the biomolecule in the sample. In some embodiments, biomolecule may be a polypeptide or protein and the system can be used to indirectly detect the presence of the biomolecule in the sample. In some embodiments, molecule or biomolecule may be a small molecule including, but not limited to, a chemical compound, a drug or a metabolite, and the system can be used to indirectly detect the presence of the molecule or biomolecule in the sample.

In some embodiments, the biomolecule is a biomarker of a disease or disorder. Biomarkers that can be detected using the systems and methods of the invention include, but are not limited to, viral nucleic acid molecules, bacterial nucleic acid molecules, a microRNA molecule, an mRNA molecule, an alternatively spliced mRNA molecule, a nucleic acid molecule harboring a disease-associated mutation, and other biomarkers associated with a disease or disorder.

In one embodiment, the invention provides methods of detecting an RNA molecule of interest. In one embodiment, the method comprises the steps of a) contacting an RNA molecule of interest with an epitape molecule comprising a probe site comprising a DNA sequence which is complementary to a region of the RNA molecule of interest, b) contacting the RNA molecule:epitape complex with an epiprinter molecule, c) allowing a cycloaddition reaction to occur between the epiprinter molecule and the epitape molecule, and d) detecting the epiprinter:epitape complex using a nanopore system.

In one embodiment, the invention provides methods of detecting a DNA molecule of interest. In one embodiment, the method comprises the steps of a) contacting a DNA molecule of interest with an epitape molecule comprising a probe site comprising an RNA sequence which is complementary to a region of the DNA molecule of interest, b) contacting the DNA molecule:epitape complex with an epiprinter molecule, c) allowing a cycloaddition reaction to occur between the epiprinter molecule and the epitape molecule, and d) detecting the epiprinter:epitape complex using a nanopore system.

In some embodiments, the methods of the invention can be used to indirectly detect a peptide or protein of interest through detection of a DNA or RNA molecule that is released in the presence of the peptide or protein of interest. For example, in one embodiment, an antibody, or antibody fragment with high affinity for a protein or peptide of interest is covalently linked to a nucleic acid molecule which can be released from the antibody by a cleavable bond (e.g. ester linkage or disulfide linkage) upon binding of the antibody to its antigenic target. The epiprinter system of the invention is then used to detect the released nucleic acid molecule, which serves as an indicator of the presence of the protein or peptide of interest.

In some embodiments, the methods of the invention can be used to indirectly detect a small molecule of interest through detection of a DNA or RNA molecule that is released in the presence of the small molecule of interest. For example, in one embodiment, an aptamer specific for binding to the small molecule of interest is covalently linked to a nucleic acid molecule which can be released from the aptamer by a cleavable bond (e.g. ester linkage or disulfide linkage) upon binding of the aptamer to its target. The epiprinter system of the invention is then used to detect the released nucleic acid molecule, which serves as an indicator of the presence of the small molecule of interest. In various embodiments, the small molecule of interest may be a chemical compound, drug or metabolite.

The systems and methods of the invention may also be used to diagnose a mammal with a disease or disorder. Some embodiments of the invention provide a method for diagnosing a mammal with a disease or disorder, comprising:

a) detecting the presence of a nucleic acid biomarker of interest in a sample obtained from the mammal, wherein the presence of the nucleic acid biomarker of interest is associated with the disease or disorder, the method of detecting comprising:

1) contacting the sample with an epitape comprising a probe site with a probe strand comprising a nucleotide sequence complementary to a region of the biomarker of interest, wherein when the biomarker of interest is an RNA molecule, the probe strand comprises a DNA molecule, wherein when the biomarker of interest is a DNA molecule, the probe strand comprises an RNA molecule, such that the biomarker of interest hybridizes to the probe site of the epitape molecule forming an RNA:DNA hybrid;

2) contacting the hybridized epitape: biomarker of interest with an epiprinter, whereby the epiprinter undergoes a cycloaddition reaction with the epitape molecule, becoming covalently linked to the epitape molecule;

3) translocating the covalently linked epiprinter-epitape molecule through a nanopore, whereby the covalently linked epiprinter-epitape molecule transiently blocks the electrical signal as it passes through the nanopore; and

4) measuring the electrical current in the nanopore system, wherein a decrease in electrical current as compared to a control indicates the presence of the biomarker of interest in the sample; and

b) diagnosing the mammal with the disease or disorder when the presence of the associated biomarker is detected.

In one embodiment, the disease or disorder is a viral infection. Thus, certain embodiments of the invention provide, a method for diagnosing a mammal with a viral infection comprising:

a) detecting the presence of a viral RNA molecule in a sample obtained from the mammal by:

1) contacting the sample with an epitape comprising a probe site with a DNA probe strand comprising a nucleotide sequence complementary to a region of a viral RNA molecule, such that the viral RNA molecule hybridizes to the DNA probe site of the epitape molecule;

2) contacting the hybridized epitape:viral RNA molecule with an epiprinter, whereby the epiprinter undergoes a cycloaddition reaction with the epitape molecule, becoming covalently linked to the epitape molecule;

3) translocating the covalently linked epiprinter-epitape molecule through a nanopore, whereby the covalently linked epiprinter-epitape molecule transiently blocks the electrical signal as it passes through the nanopore; and

4) measuring the electrical current in the nanopore system, wherein a decrease in electrical current as compared to a control indicates the presence of a viral RNA molecule; and

b) diagnosing the mammal with a viral infection when the presence of the viral nucleic acid is detected.

In certain embodiments, the methods of the invention further comprise administering a treatment or therapeutic agent to the diagnosed mammal. As used herein, the term “therapeutic agent” includes agents that provide a therapeutically desirable effect when administered to an animal (e.g., a mammal, such as a human). The agent may be of natural or synthetic origin. For example, it may be a nucleic acid, a polypeptide, a protein, a peptide, or an organic compound, such as a small molecule. The term “small molecule” includes organic molecules having a molecular weight of less than about, e.g., 1000 amu. In one embodiment a small molecule can have a molecular weight of less than about 800 amu. In another embodiment a small molecule can have a molecular weight of less than about 500 amu.

In certain embodiments, the treatment or therapeutic agent is an anti-viral agent. In certain embodiments, the treatment or therapeutic agent is an agent to treat or prevent a comorbid condition or complication of a virus.

In certain embodiments, the viral nucleic acid is from dengue virus, Ebola virus, human immunodeficiency virus (HIV), hepatitis B, hepatitis C, Influenza, Powassan virus, SARS, severe acute respiratory syndrome coronavirus 2 (SARS-CoV-2), measles, Zika, yellow fever, West Nile fever, smallpox, Marburg viruses, human papillomavirus, Kaposi's sarcoma-associated herpesvirus or human T-lymphotropic virus and the anti-viral agent is useful for treating the particular viral infection. In certain embodiments, the viral infection is caused by a dengue virus and the treatment, therapeutic or anti-viral agent is useful for treating dengue virus fever, or a complication or comorbidity thereof. Exemplary treatments or therapeutic agents useful for treating dengue virus fever, include, but are not limited to, oral rehydration regimens and intravenous (IV) fluid therapy.

Therefore, in various embodiment, the methods of the invention may further comprise administering a therapeutic agent to a mammal (e.g., a mammal diagnosed with a particular disease, disorder or condition using a method described herein). In one embodiment, the invention may further comprise administration of an agent for treatment or prevention of one or more diseases or disorders associated with a viral infection. For example, in on embodiment, the invention may further comprise administration of an anti-viral agent, pre-exposure prophylaxis (PrEP), or a medication to reduce one or more symptom associated with a viral infection. Such a therapeutic agent may be formulated as pharmaceutical composition and administered to a mammalian host, such as a human patient in a variety of forms adapted to the chosen route of administration, e.g., orally or parenterally, by intravenous, intramuscular, topical or subcutaneous routes.

### Biological Sample

The biological sample can be any sample from which the biomolecule of interest can be obtained. The biological sample(s) can be prepared using methodologies well known in the art such as by obtaining a specimen from an individual and, if necessary, disrupting any cells contained therein to release a biomolecule of interest. In one embodiment, the sample is RNA isolated from a cell or a tissue sample.

Any nucleic acid sample may be used in practicing the present invention, including without limitation eukaryotic, prokaryotic and viral nucleic acid. In one embodiment, the target nucleic acid represents a viral RNA in a sample isolated from a patient. In one embodiment, the target nucleic acid represents a viral RNA in a sample isolated from a host organism, including, but not limited to, insect host organisms such as mosquitoes, ticks or other disease vector insects.

Biological samples which can be tested by the methods of the present invention described herein include human cells, tissues and body fluids such as whole blood, serum, plasma, cerebrospinal fluid, sputum, bronchial washing, bronchial aspirates, urine, lymph fluids and various external secretions of the respiratory, intestinal and genitourinary tracts, tears, saliva, milk, white blood cells, myelomas, buccal cells, cervicovaginal cells, epithelial cells from urine, fetal cells, or any cells present in tissue obtained by biopsy and the like; biological fluids such as cell culture supernatants; tissue specimens which may be fixed; and cell specimens which may be fixed.

Biomolecules of interest may be obtained from any cell source, tissue source, or body fluid. Non-limiting examples of cell sources available in clinical practice include blood cells, buccal cells, cervicovaginal cells, epithelial cells from urine, fetal cells, or any cells present in tissue obtained by biopsy. Body fluids include blood, urine, cerebrospinal fluid, semen and tissue exudates at a site of infection or inflammation. RNA is extracted from the cell source, tissue source, or body fluid using any of the numerous methods that are standard in the art. It will be understood that the particular method used to extract RNA will depend on the nature of the source.

### Kits

The present invention further provides kits for practicing the present methods. Accordingly, certain embodiments of the invention provide a kit for detecting a biomolecule of interest in a sample comprising:

a) an epiprinter molecule;

b) an epitape molecule, or nucleic acid molecules for self-assembly of an epitape molecule, specific for detecting the biomolecule of interest;

c) reagents necessary for a cycloaddition reaction between the epiprinter and epitape molecule; and

d) instructions for use;

wherein the epiprinter will undergo a cycloaddition reaction with the epitape molecule upon epitape hybridization with the biomolecule of interest or hybridization with a nucleic acid molecule released in the presence of the biomolecule of interest; and wherein the covalently linked epiprinter:epitape complex will block electrical current through a nanopore.

In certain embodiments, the kit comprises a nanopore system as described herein for use in detecting the presence of the biomolecule. In one embodiment, the kit comprises a nanopore chip, nanopore cartridge or nanopore membrane for use in a nanopore system as described herein.

In some embodiments, the kit may optionally contain one or more of: a positive and/or negative control, materials for isolation and preparation of a nucleic acid sample (e.g., RNase-free water, and one or more buffers), and RNase-free laboratory plasticware (e.g., a plate(s), such a multi-well plate(s), such as a 96 well plate(s), a petri dish(es), a test tube(s), a cuvette(s), etc.).

Any kit of the invention may also include suitable storage containers, e.g., ampules, vials, tubes, etc., for each reagent disclosed herein. The reagents may be present in the kits in any convenient form, such as, e.g., in a solution or in a powder form. The kits may further include a packaging container, optionally having one or more partitions for housing the various reagents or components.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Development of a Click Chemistry-Reactive, High Affinity RNA-DNA Hybrid Recognition Module (Epiprinter)

The invention described herein, termed epiprinter, is a functionally versatile molecular entity that can detect RNA-DNA hybrids with potentially high sensitivity and selectivity, thus providing the basis for the proposed technology. While the epiprinter technology is anticipated to be a feature of standard clinics and laboratories, it is especially designed for use in resource-limited settings, for which the rapidity, portability, and cost-effectiveness of the technology are essential features. Epiprinter technology affords a new way to detect RNA without requirement for enzyme or a nucleic acid amplification step, as is the case for example with polymerase chain reaction (PCR)-based technologies. The epiprinter, used in conjunction with custom DNA nanostructures and solid-state nanopores, is also designed to provide multiplexed detection of RNA. In this regard, nanopore-related technologies, which will include epiprinting, can provide high throughput, single-molecule detection capability. It will have portability for use in diverse settings, and without the need for highly trained personnel. Epiprinter detection of RNA also is designed to be nondestructive to the RNA sample, allowing further analysis of the RNA as needed.

With simple modification the epiprinter may be used to detect RNA-DNA hybrids in cellulo and in situ, with high sensitivity and accuracy. With other specific modifications, the epiprinter can be used for detection of DNA, or detection of protein, with the latter in conjunction with antibodies or aptamers. In summary, epiprinter technology intends to provide sensitive, accurate, rapid, and convenient detection of biomolecules in virtually any setting, taking advantage of the high specificity achieved by the association of complementary RNA and DNA sequences, the accessibility and functionality of custom-designed DNA nanostructures, a robust and elective chemical reaction, and the high throughput, single-molecule detection capability of solid-state nanopore-based technologies.

The specificity, high affinity, and chemical functionality of the epiprinter provides essential contributions to new and better ways to detect RNA and RNA-DNA hybrids, with broad medical, biotechnological, agricultural, and basic research applications. In this respect the epiprinter technology for RNA detection intends to be readily adaptable to ongoing advances in solid-state nanopore technology, to provide portability as well as near-real-time RNA detection. The epiprinter method is enzyme- and label-free, and provides high-throughput, single-molecule readout with multiplexing capacity (for example, via nanopore analysis) that permits sensitive and accurate detection of multiple RNAs in a wide range of physical settings. Such a point-of-need (PON) technology allows earlier prediction of, and rapid response to, emerging disease outbreaks.

The epiprinter technology is well-aligned with a set of criteria developed for optimal PON diagnostics as proposed by the World Health Organization (WHO). Specifically, the ASSURED (affordable, sensitive, specific, user-friendly, rapid & robust, equipment-free, and deliverable to end users) criteria for an effective diagnostic test, as originally proposed for HIV detection in resource-limited regions (Peeling and Mabey 2010; Wu and Zaman 2012) is also applicable to other pathogen identification methods in remote locations as well as in standard laboratory or clinical settings. The multiplexed detection of RNA enables rapid identification of the pathogen(s) among the numerous possibilities, thus providing precision diagnosis and informing the best course of treatment. Multiplexing is a programmed capability of the epiprinter technology. This capability meets the challenge of diagnosis of acute febrile illness (AFI) that can reflect an infection by any one of a number of viruses (Robinson and Manabe 2017). Multiplexing also improves field surveillance of insect vectors. As described below, the epiprinter technology also can be modified to detect DNA, protein, or small molecules. Moreover, an epiprinter could simultaneously detect RNA, DNA, proteins, and small molecules, thus providing important multiplexed detection of multiple types of biomolecules and molecules. It is important to note that while the focus here has been on detecting viral RNAs, the epiprinter technology also can detect any other type of RNA. As such, it can be applied in basic and clinical research in the identification of RNA present in cells and tissues in humans, livestock, plants, insects, food and water sources.

Epiprinting technology can be distinguished from nanopore-based RNA sequencing approaches in that it involves the targeted detection of RNA (detection by capture), rather than whole RNA sample analysis that includes generation of a very large file, representing the entire sequence population. In contrast to whole sample analysis, epiprinter capture detection places less of a premium on nanopore sequencing capacity and computational capacity. While whole sample analysis is important for genomic and transcriptomic analyses, the epiprinter technology focuses on detecting only the RNAs of interest—crucial for diagnostic tests—by virtue of selective RNA capture by an epitape, followed by nanopore analysis only of the epitapes.

Epiprinter Technology

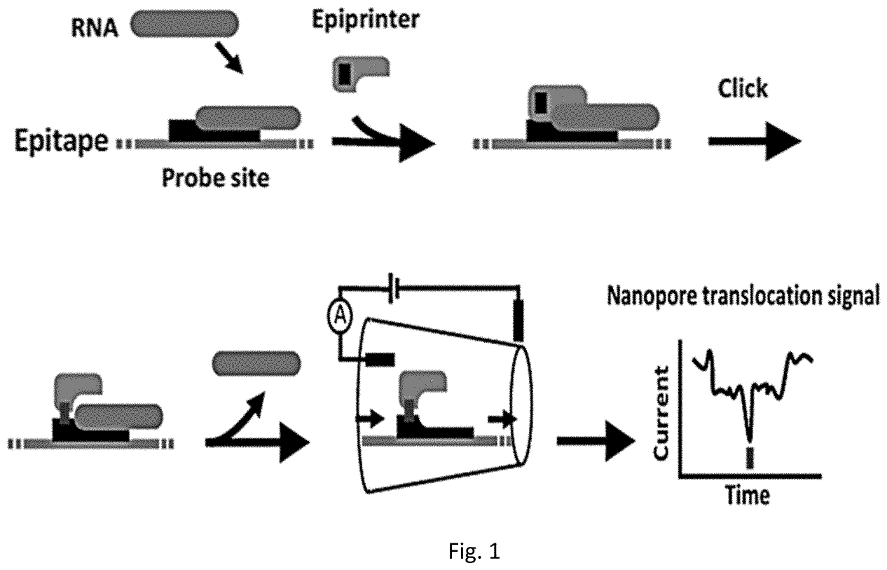

The term “epiprinter” was chosen to reflect the basic mode of action of the device. Specifically, epiprinter binding to an RNA-DNA hybrid is followed by covalent modification of the DNA strand of the hybrid (FIG. 1). This modification is analogous to DNA covalent modifications such as base methylation that occur in cellular epigenetic regulatory processes. The epiprinter is a modified, synthetic protein construct that specifically recognizes RNA-DNA hybrids with high affinity and selectivity. Epiprinter detection of RNA exploits the inherent ability of an RNA molecule to spontaneously associate with a DNA molecule of complementary nucleotide sequence, forming a stable, double-helical RNA-DNA hybrid structure. Thus, the epiprinter detects RNA as a consequence of RNA-DNA hybrid formation.

The sequence-specificity of RNA detection is a natural outcome of the requirement for sequence complementary in forming a stable RNA-DNA hybrid. The epiprinter contains multiple copies of a polypeptide termed the hybrid-binding domain (HBD). The HBD is a conserved structural domain present in ribonuclease H1, an enzyme that cleaves the RNA strand of hybrids that are generated during specific cellular processes (Cerritelli and Crouch 2009). The HBD binds hybrids in a noncovalent, reversible manner, with a binding affinity (as measured by the K_{D}) in the ˜submicromolar range (Jongruja et al., 2010, FEBS Journal, 277: 4474-4489; Nowotny et al., 2008, The EMBO Journal, 27: 1172-1181). HBD binding to hybrids is independent of hybrid base-pair sequence. This sequence non-specificity allows the HBD—and therefore also the epiprinter—to detect any RNA of any sequence, as engaged in a stable hybrid with a complementary DNA. In addition, the HBD of human RNase H1 has high selectivity for hybrids as it exhibits negligible affinity for single-stranded DNA, double-stranded DNA (˜100 times less than the hybrid), single-stranded RNA, and only a very low affinity for double-stranded RNA (˜25 times less than the hybrid) (Cerritelli and Crouch 2009). This selectivity is essential for enabling the epiprinter to detect a specific RNA in the presence of the other types of nucleic acids, as typically occurs in biological samples.

The structural basis for hybrid specificity is revealed by a crystallographic analysis of the HBD from human ribonuclease H1 bound to a 12-bp hybrid (Nowotny et al., 2008, The EMBO Journal, 27: 1172-1181). The HBD recognizes successive 2′-hydroxyl groups on the RNA strand and successive 2′-deoxyribose residues on the opposing DNA strand. This structural arrangement only occurs in an RNA-DNA hybrid. The HBD directly contacts 5 contiguous bp of hybrid structure, but physically spans approximately 9 bp. Thermodynamic principles predict that linking multiple copies of RNA-binding protein domains confer an RNA binding enhancement that can approach the thermodynamic limit of full additivity of the binding free energy of the individual domains (Shamoo et al. 1995). In fact, the presence of multiple copies of the HBD in the epiprinter confer subnanomolar (K_{D}) binding affinity for hybrids compared to that of the monomeric HBD.

The binding affinity of the THBD, containing three copies of the HBD equals, if not surpasses, that of other molecular entities that recognize hybrid structures, including the S9.6 monoclonal antibody (Boguslawski et al. 1986) and the small molecule conjugate, methidium-neomycin (Shaw et al. 2008). Achieving subnanomolar binding affinity is essential for high-sensitivity detection of trace levels of RNA as would be frequently encountered in the laboratory, clinic, or field. The epiprinter carries an additional modification that allows its covalent attachment, in a controlled manner, to the DNA strand of the hybrid. The covalent attachment provides a permanent “mark” on the hybrid—analogous to an epigenetic modification of DNA—that registers the prior binding of the RNA to form the hybrid (FIG. 1).

Epitape

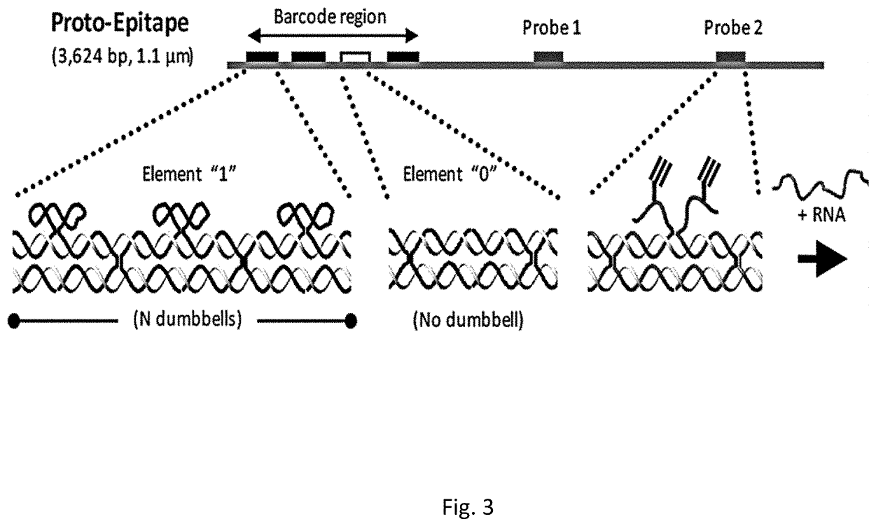

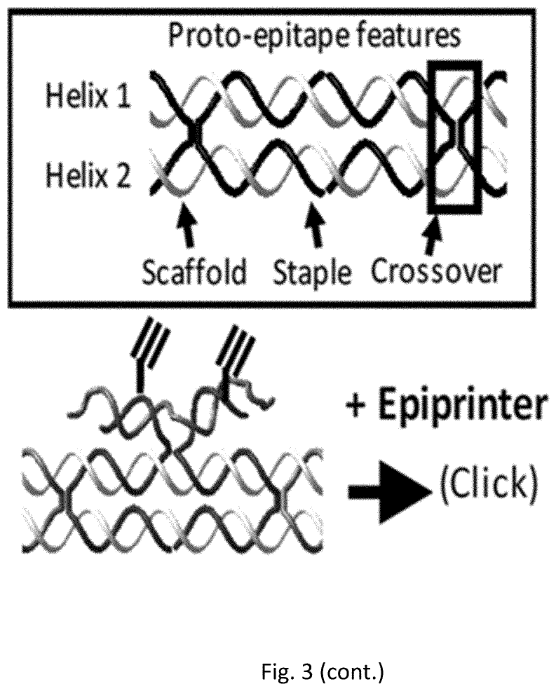

The epiprinter is intended to function with a custom DNA nanostructure that can bind one or more RNAs at specified locations (probe sites), forming localized hybrid structures. Each probe site in the nanostructure has a sequence complementary to an RNA target sequence of interest. Reflective of this function, the nanostructure is termed an epitape (FIG. 3). Similar DNA nanostructures capable of forming hybrids were already shown to function in RNA detection in cell lysates, in conjunction with atomic force microscopy (AFM) imaging (Ke et al. 2008). This study and other reports (Stopar et al. 2018; Goltry et al. 2015) also showed that DNA nanostructures are resistant to nucleolytic degradation in biological fluids, including serum. In this protocol, a sample of RNA purified from a specific source (e.g. blood, insects), is combined with the epitape, allowing the target RNA (if present) to bind to the complementary probe site, creating a localized hybrid structure. For low abundance RNAs, longer incubation times would ensure formation of the hybrid. By using an excess of epitape to interrogate the RNA sample, the detection output signal would be proportional to the amount of target RNA in the sample. Two terminal alkynyl groups are on the DNA strands of probe site 2. The RNA binds to probe site 2 in a manner similar to that described elsewhere (Ke et al. 2008). The barcode region shows two DNA secondary structural elements (“dumbbells”), allowing identification of the epitape. A digital (1/0) identifier is provided by the two barcode elements.

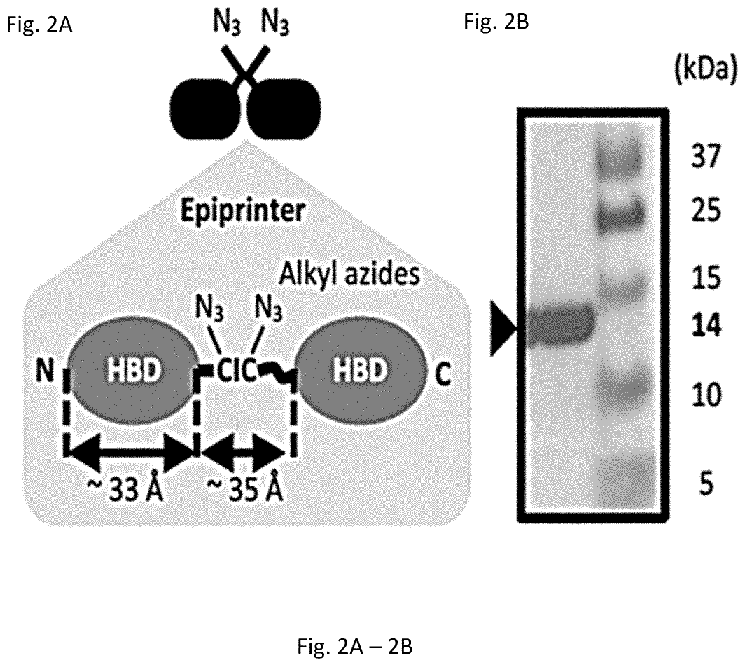

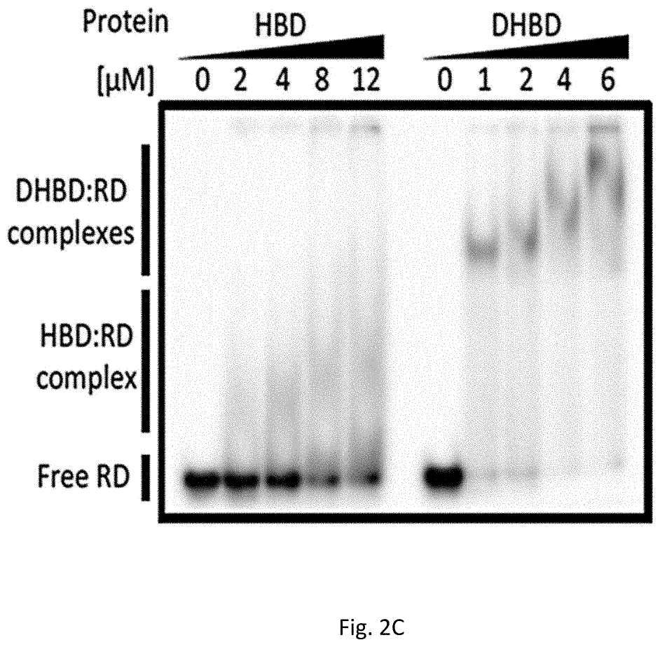

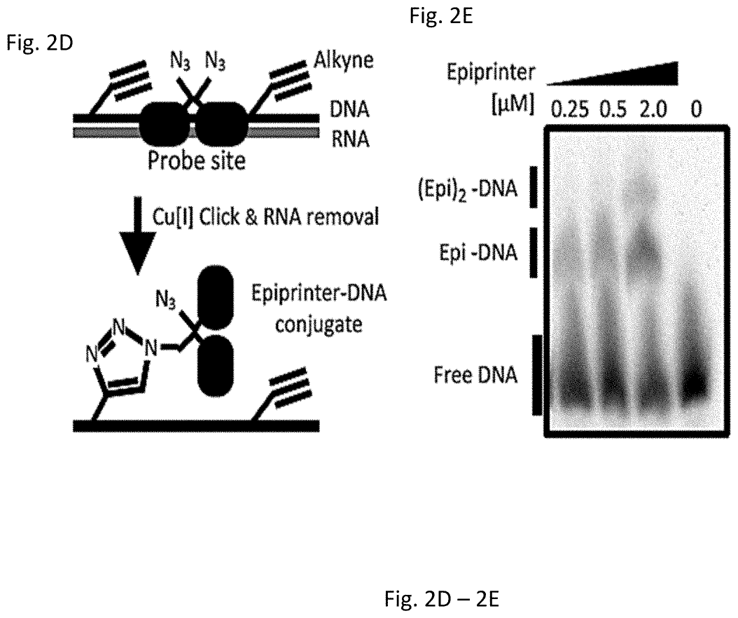

FIG. 2 shows the interaction of the v.1 epiprinter with the alkynyl DNA-RNA hybrid. To allow their facile manipulation, the epitapes can be attached to magnetic beads by a cleavable linker affixed to specific DNA sequence in the epitape. Following incubation with the RNA sample, the epitapes can be collected by application of a magnetic field, then gently washed to remove the remainder of the sample. After resuspension of the epitape, “click” reaction reagents are then added, catalyzing permanent covalent attachment of the epiprinter to the epitape at the hybrid-containing probe site(s). The epitapes are then washed to remove reaction components, then released from the magnetic beads by cleavage of the linker using a mild chemical reaction (e.g., hydrolysis or reduction).

The epitapes released into solution are then subjected to nanopore readout analysis. If the RNA that binds to a probe site is large, then a chemical hydrolysis (pH 9 carbonate/bicarbonate buffer) can size-reduce the RNA to provide a hybrid-carrying epitape amenable to nanopore translocation. Alternatively, if the bound RNA is important to retain for further analysis, it can be released from the epitape following click reaction by mild heating in the presence of a biocompatible organic solvent or solute (e.g. formamide or urea). The RNA can be recovered from solution, and the epitape released from the beads for nanopore analysis. Nanopores subjected to a voltage potential allow ion current flow, typically in the picoampere range (Bell et al., 2013, Lab on a Chip, 13:1859). DNA also can be translocated across nanopores of sufficient diameter, reflecting its inherent negative charge (Bell and Keyser, 2016, Nature Nanotechnology, 11: 645-651).

During DNA translocation there is a transient diminution of current flow, reflecting the steric bulk of the translocating DNA. When a translocating epitape carries a probe-site-attached epiprinter, the sterically bulky structure causes an additional, superimposed transient drop in current that can be recorded. Modulation of nanopore current flow by sterically bulky ligands attached to DNA has been demonstrated elsewhere (Bell and Keyser 2015; Plesa et al. 2015). The barcode structures in the epitape (FIG. 3) also are designed to modulate ion current, such that when an individual epitape translocates across a nanopore it is identified as to its probe site RNA specificities. The epitape diagram shown in FIG. 3 carries two probe sites. Since the linear epitape has non-identical termini (with the barcode region at one end), and since translocation rates can be controlled to provide optimal temporal nanopore interrogation of the probe sites and barcodes, the epitape can be accurately identified, as well as scored for probe site RNA binding status. Irrespective of which end is the leading end during translocation, an accurate analysis is obtainable.

One of the epitape probe sites can provide a built-in internal (positive) control for proper performance of the epiprinting process. Here, a well-characterized RNA (for example, MS2 phage RNA (Dreier et al. 2005)) is added in a specified amount to the RNA sample. The probe site carries a sequence complementary to a unique sequence in the MS2 RNA. Nanopore analysis should detect a positive signal at this probe site, with a detection frequency proportional to the amount of RNA added. For accurate and sensitive detection of RNA, the epitape will employ probe sites that create hybrids of 35-50 bp in length (Geiss et al. 2008), since hybrids of shorter length would pose a progressively increasing probability of non-uniqueness in RNA recognition, and therefore reduce the specificity by enabling the binding of “off-target” RNAs. RNA-DNA hybrid stability is also dependent upon hybrid length, with shorter hybrids less stable than longer ones (Lesnik and Freier 1995). With these considerations the epiprinter technology is able to take optimal advantage of detecting hybrid structures with lengths >35 bp. A hybrid of this length can accommodate the binding of 3-4 copies of the v.1 epiprinter (containing two HBDs), or 1-2 copies of the v.2 epiprinter (three HBDs). The enhancement of steric bulk provided by multiple copies of the epiprinter linked to an epitape probe site enhances the transient drop in ion current during translocation, providing a more robust output signal.

Epiprinting Quantification Limits.

Keyser and co-workers (Kong et al., 2016, Nano Letters, 16: 3557-3562) characterized the ability of a solid-state (quartz) nanopore-based system to quantitate protein. They used a DNA carrier with a probe site with affinity for the protein target, then used a solid-state (quartz) nanopore to detect the protein bound to the DNA carrier by its ability to modulate ion flow through the nanopore.

In detecting an antibody with a binding affinity (K_{D}) of ˜3.5 nM, the lowest concentration of antibody able to be detected was ˜3 nM. This limit reflected (i) the affinity (K_{D}) between the target and probe molecules; (ii) the DNA carrier: protein concentration ratio; and (iii) the molecular weight of the target protein (the greater the molecular weight, the higher the signal-to-noise ratio). While these parameters can be modified to improve the detection limit, the most difficult to improve is the probe-to-target affinity. An epiprinter with three HBDs (THBD) with a measured K_{D} of ˜0.2 nM for hybrid binding, allows detection of an RNA in the sub-nanomolar concentration range. Regarding parameter (ii), an excess of epitape over target RNA can improve the sensitivity, since an increase in receptor concentration can drive the equilibrium towards essentially complete binding of the target molecule(s). This would also provide an output signal range that is proportional to the amount of target RNA in the sample. The biotin-streptavidin interaction exhibits the strongest non-covalent binding affinity known to date (K_{D}˜10⁻¹⁵ M). This interaction therefore is expected to provide the lowest detection limit (highest sensitivity) for a noncovalent probe-target interaction. However, formation of a covalent bond between probe and target, as accomplished by click chemistry in the epiprinting technology, further lowers the detection limit.

Sample Processing and RNA Purification.

Samples to be analyzed can be combined with RNA-protective solutions such as RNAlater™ (ThermoFisher). The RNA can then be isolated using a guanidinium thiocyanate-based RNA extraction reagent (e.g., TRIzol™, ThermoFisher) (Depledge et al. 2019). Sample preparation can be automated with existing instruments such as the VolTRAX V2 (Oxford Nanopore Technologies, UK) or PDQeX (MicroGEM, UK). Automated, portable sample preparation should reduce sample preparation time, provide consistency in RNA quality, reduce reagent amount, and obviate the requirement for a trained operator.

Click-Chemistry Reaction.

Copper-catalyzed Azide-Alkyne Cycloaddition (CuAAC) is a chemistry used in bioconjugation reactions involving proteins, nucleic acids, polysaccharides, and lipids both in vitro and in vivo. Its popularity of use reflects the rapid reaction rate at room temperature (second order rate constant ˜10-200 M^{-1 s-1} per 10-100 μM Cu[I]), as well as its intrinsic biorthogonality and high selectivity (Hong et al. 2009, 2010; McKay and Finn 2014). CuAAC chemistry can catalyze in situ conjugations between chemical molecules juxtaposed with the help of a scaffold such as the ribosome (Glassford et al. 2016) or DNA strands forming a double helix (Jacobsen et al. 2010). While the toxicity of the Cu(I) catalyst creates an incompatibility for the reaction within living cells (Hong et al. 2010), this is not an issue for the epiprinter technology that is designed to function in cell lysates (an ex-vivo form of the cells) as well as with purified nucleic acid samples. Nonetheless, in the CuAAC reaction, the Cu(I) ion and reducing agent can generate reactive oxygen species (ROS) that can chemically damage biomolecules. If not carefully controlled, ROS generation could affect epiprinter and epitape performance. The inclusion of specific Cu-binding ligands, such as Tris(3-hydroxylpropyltriazolylmethyl)amine (THPTA) and aminoguanidine reduces any deleterious effect of Cu(I) ions to a negligible level. Addition of Cu(I)-binding ligands accelerates the CuAAC reaction rate (Besanceney-Webler et al., 2011, Angewandte Chemie International Edition, 50:8051-8056), and an optimized protocol was developed for CuAAC bioconjugation reactions in aqueous media (Hong et al. 2009). Specifically, inclusion of THPTA and aminoguanidine can protect (i) histidine, (ii) a protein, and (iii) a short dsRNA (21 bp) for up to 1 hour from Cu(I)-dependent oxidative damage (Hong et al. 2009). The chemical stability of the biomolecules involved in the epitape/epiprinter procedure is not a concern as the reaction is completed within 15 minutes. CuAAC chemistry with optimized protocols was shown to function in an even more complex context; namely, in whole protein solution for cell-surface labeling, in cell culture, and in living zebrafish embryos (Speers et al. 2003; Yang et al. 2013; Hong et al. 2010; Besanceney-Webler et al., 2011, Angewandte Chemie International Edition, 50:8051-8056). In applying the CuAAC chemistry to the epiprinter RNA detection technology, the sensitivity and signal-to-noise ratio are optimized, while any Cu(I)-dependent biomolecule damage is minimized using the approaches mentioned above. Background nonspecific reactions resulting in reduced sensitivity may be a drawback of CuAAC chemistry, but several reports demonstrated that these problems can be addressed by Cu-ligand screening, changing the Cu:Cu-ligand ratio, or exchanging the positions of the click-reactive groups in the epiprinter and epitape (Speers and Cravat 2004; Hong et al. 2009; Besanceney-Webler et al., 2011, Angewandte Chemie International Edition, 50:8051-8056). An alternative, Cu(I)-independent, Strain-Promoted Azide-Alkyne Cycloaddition (SPAAC) chemistry (Agard et al. 2004) can also be applied. Here, the epitape probe site carries Bicyclo[6.1.0] nonyne (BCN) groups attached to specific probe site dU residues. Binding of the RNA, followed by epiprinter addition allows spontaneous, Cu(I)-independent azido-BCN cycloaddition. The epitape is then subjected to nanopore analysis as described above. The BCN structural analog, biarylazacyclooctynone (BARAC), can also be used, as it is less toxic to cells (Jewett et al. 2010). Finally, ongoing efforts in developing SPAAC chemistry is expected to enhance the reaction kinetics (McKay and Finn 2014).

Construction and Purification of the HBD

The HBD is from ribonuclease H1 of the bacterium Thermotoga maritima. The HBD (and therefore also the epiprinter) has durable function under a variety of conditions, since T. maritima is a hyperthermophilic organism, with the encoded proteins exhibiting thermostability (Lesley et al. 2002; Nathania and Nicholson 2010; Shi et al. 2011; Meng et al. 2008). The HBD DNA sequence was cloned from the T. maritima genomic DNA (obtained from American Type Culture Collection, ATCC) using PCR and gene-specific primers. The resulting amplified DNA fragment carries an NdeI site at one end and a BamHI site at the other end. The DNA fragment was digested with BamHI and NdeI, then ligated into the pET15b plasmid that also had been digested with BamHI and NdeI. The HBD plasmid was overexpressed in E. coli 10Beta cells (New England Biolabs) and then purified using a plasmid purification kit (Qiagen). The T. maritima RNase H1 HBD, also carrying a thrombin-removable N-terminal hexahistidine (H6) affinity tag, was PCR-cloned and produced in E. coli BL21(DE3) cells. The protein was purified from sonicated cell extracts by Ni-NTA affinity chromatography. The H6 tag was removed by thrombin cleavage, followed by affinity and size exclusion chromatographic steps, providing the HBD with >95% purity. SDS-PAGE analysis revealed a single polypeptide with a gel electrophoretic mobility consistent with the its calculated molecular mass (6,730 Da).

Gene Construction and Purification of the Double HBD (DHBD).

Golden Gate Assembly (GGA) (Engler et al., 2008, PLoS ONE, 3:e3647) was used to assemble a DNA encoding the double(d) HBD. The DNA sequence encoding the T. maritima HBD was the starting point. Two HBD DNAs were designed to be joined by the same linker in T. maritima RNase H1 that connects the HBD to the C-terminal catalytic domain. An alternative version of the dHBD also was constructed in which the 1st and 3rd cysteine residues (-CIC-) in the linker were mutated to serine (-SIS-). The dHBD gene sequence was incorporated into two dsDNA constructs that were synthesized and purified by IDT, Inc. (Coralville, Iowa, USA). The following were combined in a single-pot reaction: (i) a modified pET15b plasmid (previously engineered to remove the single BsaI restriction enzyme site, and to carry a new BsaI site in the multicloning segment) and the two synthetic dsDNA fragments. BsaI restriction enzyme and T4 DNA ligase were added in buffer containing ATP to provide a 3-way ligation reaction. The action of BsaI (a type IIS restriction endonuclease) provided the proper end overhangs that would correctly orient the two inserts in the modified pET15b plasmid. Recombinant plasmids were isolated and the correct insert sequence verified by sequencing (GeneWiz). The dHBD plasmid was amplified in E. coli 10Beta competent cells (New England Biolabs) and then purified using a plasmid purification kit (Qiagen). The recombinant dHBD protein was produced in the E. coli expression host, BL21(DE3) and purified by affinity chromatography, as described above for the HBD. Mass spectroscopic analysis and SDS-PAGE analysis of the purified protein both were consistent with the calculated molecular mass of the dHBD (14,233 Da).

Construction and Purification of the Triple HBD (tHBD).

The Golden Gate Assembly (GGA) also was used to prepare a DNA sequence encoding the triple HBD. The T. maritima HBD DNA sequence was the starting point. The HBD DNA sequences also were designed to be connected by the same linker that connects the HBD to the C-terminal catalytic domain of T. maritima RNase H1. For both linkers the 1st and 3rd cysteine residues (CIC) were changed to serine (SIS). The tHBD gene sequence was divided between two dsDNA constructs. The modified pET15b plasmid (modified for use with BsaI restriction enzyme—see above), the three synthetic dsDNA fragments, BsaI restriction enzyme and T4 ligase were combined in a single-pot reaction. BsaI was used to produce the proper cohesive ends that provided correct orientation of the inserts in the modified pET-15b vector, in a four-way ligation reaction. The correct sequence of an isolated recombinant plasmid was verified by DNA sequencing. The THBD plasmid was amplified in E. coli 10Beta competent cells (New England Biolabs), then purified using a purification kit (Qiagen). The encoded THBD was produced in the E. coli expression host BL21(DE3), and was purified by affinity chromatography as described above for the DHBD. SDS-PAGE analysis provided a mobility consistent with the calculated molecular mass of the THBD (21,737 Da) (FIG. 3).

Epiprinter Covalent Attachment to an RNA-DNA Hybrid (“Epiprinting”)

To create a version 1 (v.1) epiprinter, the two next-nearest-neighbor cysteines in the DHBD linker were conjugated to alkyl chains carrying terminal azido groups, using maleimide coupling chemistry (Kantner et al. 2017). The conjugation allows covalent attachment of the epiprinter to the (modified) DNA strand of an RNA-DNA hybrid, using “click” chemistry. Chromatography provided purified v.1 epiprinter, and the near-stoichiometric alkylation of both cysteines was verified by mass spectroscopic analysis. The ability of the v.1 epiprinter to covalently attach to an RNA-DNA hybrid was examined as follows. A modified 21 bp RNA-DNA hybrid was prepared in which two thymines in the DNA strand (termed DNA*) were replaced by deoxyuridine (dU) residues, both of which carried on the 5-carbon hexyl chains with terminal alkynyl groups. Binding of the v.1 epiprinter to the RNA-DNA* hybrid places the alkylazido groups of the v1. epiprinter in close proximity to the DNA* alkynyl groups. Addition of Cu(I) ions then catalyzes a click reaction of the alkylazido and alkynyl groups, creating a chemically stable triazole (Presolski et al. 2011), thus covalently linking the v.1 epiprinter to the DNA* strand (FIG. 2D). The v.1 epiprinter was combined with the RNA-DNA* hybrid (with the DNA* 5′-³²P labeled) to allow binding. Cu(I) ion was added to initiate the click reaction, which was performed at room temperature for 15 minutes. The reaction was then subjected to denaturing polyacrylamide gel electrophoresis, followed by phosphorimaging. The results (FIG. 2E) reveal a slowed electrophoretic migration of the radiolabeled DNA*, indicative of an increased mass. This product was dependent upon (i) Cu(I) ion addition; (ii) alkynyl groups on the DNA, and (iii) azido groups on the v.1 epiprinter. The control reactions confirmed the requirement for click chemistry to generate the DNA* of greater mass. This reaction did not occur with DNA* alone, thus confirming the requirement for the RNA-DNA hybrid structure for reactivity. The appearance of an additional radiolabeled product of even slower mobility (FIG. 2E) suggests that two v.1 epiprinters can covalently attach to the DNA* strand. Formation of this additional product was possible since the DNA* strand has two alkynyl groups. In support of this, a hybrid containing a DNA* with only one alkynyl group provided a single product of slower mobility. In summary, these experiments describe the ability of a prototype (v.1) epiprinter to (i) selectively recognize and bind RNA-DNA hybrids, and (ii) covalently attach to an alkynyl-modified DNA backbone by click chemistry. These reactions are the foundation for providing a permanent recording of prior RNA-DNA hybrid formation in RNA detection by epiprinting.

Detection of RNA-DNA Hybrids in Cellulo and In Situ.

RNA-DNA hybrids are formed in diverse cellular processes including DNA replication, DNA recombination, DNA repair, and transcription (Santos-Pereira and Aguilera 2015). Hybrids are also intermediates in the replicative pathways of retroviruses (Tian et al. 2018) and are biomarkers of specific disease and inflammatory conditions (Crow 2013). There is thus a need to directly detect hybrids in cells, tissues and biological fluids for basic research and clinical diagnostics. Hybrid detection currently involves the S9.6 monoclonal antibody. However, the affinity of the antibody for the hybrid can be affected by hybrid sequence, and the antibody also has significant affinity for dsRNA, necessitating prior treatment of samples to remove the dsRNA. Another study used a catalytically inactive version of the hybrid-cleaving enzyme, ribonuclease H (Chen et al. 2017). The THBD provides a high-affinity module for detection of hybrids in situ or in cellulo, in a manner independent of base-pair sequence and without appreciable “off-target” affinity for dsRNA. To achieve this, the purified THBD is modified to carry a reporter label on the same cysteine residues that carry the alklyazido groups in the v.1 epiprinter. The fluor-labeled THBD is introduced in cells for in cellulo detection of hybrids, or used in detecting hybrids in formalin-fixed, paraffin-embedded tissue samples. The small size and high affinity of the fluor-labeled THBD (compared to an antibody) would facilitate cell penetration, providing higher sensitivity as well as selectivity in detecting hybrids by fluorescence microscopy.

DNA Detection

Sequence-specific detection of DNA is required in many procedures and protocols in clinical and basic research. This would include single nucleotide polymorphism (SNP) detection and pathogen detection (Bluth and Bluth 2018), species identification (Pereira and Amorim 2008), and molecular forensics (Tao et al. 2018). A simple modification to the epiprinting technology is envisioned to enable detection of DNA sequences. Specifically, the epiprinter will work in conjunction with a modified epitape (“riboepitape”) that carries probe sites with RNA sequences (instead of DNA sequences) that are complementary to sequences in the target DNA. Specific bases (e.g. uracil) in the probe site RNA sequences will contain alkynyl groups, enabling click chemistry. Binding of a DNA with a sequence that is complementary to the riboepitape probe site RNA will create a stable hybrid. Addition of the epiprinter, followed by the click reaction provides permanent attachment of the epiprinter to the riboepitape. Solid-state nanopore analysis of the modified riboepitape can then be performed.

Protein Detection

The detection of proteins with high specificity and sensitivity provides the foundation for many clinical diagnostic tests, and is also an essential tool for basic research. Antibodies continue to provide the primary means for specific and sensitive protein identification. We propose that antibodies can be used in conjunction with a modified epitape, containing an RNA sequence instead of a DNA sequence, to provide protein detection. Purified monoclonal antibody, or antibody fragment (Fab) with high affinity for a specific protein can be covalently linked to a DNA oligonucleotide using established procedures (Maerle et al. 2019).

The DNA can be released from the antibody by a cleavable bond (e.g. ester linkage or disulfide linkage). The DNA oligonucleotide sequence is complementary to the probe site RNA sequence of the modified epitape. Samples to be analyzed for the presence of the antigen of interest are added to a 96-well plate. Addition of the antibody-DNA conjugate then allows protein-antibody recognition. A washing step removes any unbound antibody-DNA conjugate. The epitape is added, and if the antibody-DNA conjugate is present, the DNA binds to the epitape probe site, forming an RNA-DNA hybrid. A subsequent click reaction allows covalent attachment of the epiprinter to the epitape. The DNA is then separated from the antibody by cleavage (e.g. ester hydrolysis or reduction) allowing release of the epiprinter-modified epitape into solution. The epitape is then subjected to solid-state nanopore analysis, where detection of an attached epiprinter indicates a prior antigen-antibody recognition event. Multiplexed protein detection is performed in the same manner as RNA detection (see above).

In summary, this approach serves to harness the sensitivity of nanopore detection to a wide range of proteins, taking advantage of antibodies and the ability of conjugating DNA of chosen sequence to the antibody. This approach also allows detection of non-protein antigens using the appropriate antibody. Finally, aptamer technology (Nezlin 2016) can be applied to provide high affinity probes that can work in conjunction with the epiprinter and epitape, to detect a wide variety of biomolecules or molecules otherwise inaccessible to antibody-based detection.

Current RNA detection technologies include nucleic acid amplification-based approaches, such as reverse transcription coupled with polymerase chain reaction (RT-PCR), and amplification-independent, direct detection technologies, such as the Nanostring nCounter system, and nanopore-based sequencing of RNA or complementary DNA. Also, RNA detection methodologies can be operationally divided between nucleotide sequencing approaches and complementary base-pairing approaches. Epiprinting technology falls into the latter category. Provided below is a non-comprehensive list of current RNA detection methodologies with brief descriptions of their advantages and disadvantages.

RT-PCR-Based Technologies

A broadly used RNA detection approach, with multiple variations, involves the enzymatic copying of an RNA into DNA, using the enzyme reverse transcriptase (RT) and a DNA primer. The complementary(c) DNA product is then subjected to exponential amplification using DNA polymerase and a set of DNA primers, either in a temperature-cycling mode, or by isothermal approaches. The amplified DNA product (amplicon) is detected by fluorescent dye binding or other readout label. Multiplexed RNA detection can be accomplished using multiple primer pairs in PCR, producing multiple amplicons that can be differentiated and identified by size. Quantitative RT-PCR (RT-qPCR) is used for real-time cDNA detection and involves specific instrumentation allowing determination of the amount of the target cDNA in the initial sample, as based on the timing of output signal generation and the generation of standard curves (Bustin and Mueller 2005). As mentioned, enzyme-based nucleic acid amplification technologies such as PCR remain the standard for nucleic acid detection due to its high sensitivity (50 DNA copies/mL) and specificity. However, the high sensitivity of the amplification-based approaches is a double-edged sword, as unspecific products may be amplified, especially in multiplexed reactions, with the attendant possibility of false negative or false positive outputs (Potapov and Ong, 2017, PLOS ONE, 12: e0169774). Lack of specificity may derive also from the multiple reaction steps, that include one or more enzymes, which also can introduce biases in the detection output (van Dijk et al. 2014).

DNA Microarrays

DNA microarrays allow simultaneous detection of thousands of RNAs, obtained from cells or other complex biological samples (Katagiri and Glazebrook 2009; Schneider and Niemeyer 2018). The approach involves formation of a surface array (grid), consisting of many individual “cells” which contain surface-affixed, short synthetic DNA oligonucleotides that are each complementary to an RNA of interest. Each cell thus provides a probe for a different RNA. RNAs are isolated, purified, fragmented, then complementary sequences are enzymatically generated that contain (or have an affinity tag to bind) a fluorescent readout label. Hybridization of the processed sample to the microarray is followed by high resolution fluorescence detection, providing a readout of the occurrence and relative amount of each RNA. Multiple steps, and the use of enzymes, labels and ancillary equipment (e.g. Bioanalyzer) make this a relatively expensive technology, and a requirement for trained personnel. As such, it is used in resource-rich clinical and research laboratories. The method also is destructive to the RNA sample, and the sensitivity is generally lower than that achieved by RT-qPCR (Katagiri and Glazebrook 2009).

RNA Detection by Nanostring nCounter.

This is a direct, single molecule approach for the multiplexed and quantitative detection of RNAs for analysis of gene expression (Geiss et al. 2008). Purified RNAs are “captured” by a complementary DNA that also confines the RNA to the detection surface within a microfluidic flow cell channel. A second, complementary DNA (reporter probe) then binds the RNA. The reporter probe is barcoded for transcript identification using four different fluorescent dyes that can bind to one or more of seven binding sites on the barcode. In response to an applied unidirectional fluid flow in the channel, the captured RNAs become extended linear “strings” in the flow channel, allowing the characteristic fluorescent barcode sequence to be read by high resolution imaging. This technology does not involve amplification or enzymes as with RT-qPCR, but demonstrates a similar sensitivity (Geiss et al. 2008). The equipment, involving microfluidics and high-resolution fluorescence detection, is best-suited for clinical and basic research laboratories with trained personnel.

Nanopore Sequencing

Nanopores of controlled diameter can permit translocation of ions through the pores in response to a voltage potential, yielding a measurable current (at picoampere levels). The translocation of negatively charged DNA (or RNA) through the pore, also in response to the voltage potential, causes a characteristic drop in ion current due to the transient steric perturbation of the ion current. Nanopores can be further modified to provide current output change in response to the nucleotide sequence of the translocating DNA. Minute additional current modulations during the DNA translocation event can provide the DNA nucleotide sequence, using sophisticated software analyses. In this approach, the purified RNA must first be enzymatically copied to a DNA sequence. The nanopore sequencing kit as offered by Oxford Nanopore Technologies (ONT) has the advantage of portability. Virus detection by nanopore sequencing was demonstrated with the 2015 Ebola virus outbreak in Nigeria (Quick et al. 2016). Here, acquisition of sequence data with the ONT MinION system allowed on-site monitoring of virus spread and genome sequence changes, in near real-time. While nanopore-based sequencing requires several steps, including enzymatic conversion of RNA to cDNA, this study showed the portability and effectiveness of a nanopore-based device in a resource-limited setting (Quick et al. 2016). Yet, Nanopore sequencing suffers a high read level error rate (higher than second generation sequencing, e.g. Illumina® (Keller et al. 2018)), but is capable of detecting and sequencing low-to-near zero amounts of input DNA (Pontefract et al. 2018). Direct sequencing of RNA by nanopores also has been reported by Oxford Nanopore Technologies (Garalde et al. 2018). In this approach, enzymatic ligation of DNA adaptors to 3′-termini of purified RNA enabled nanopore sequencing using the MinION system. In this approach there is no prior RNA selection step, so that all RNAs are sequenced. While useful for transcriptomic analyses, the large-scale sequence analysis may hinder the efficiency needed for rapid detection and identification of pathogens.

CRISPR-Based RNA Detection

The recent introduction of CRISPR-based diagnostic tools (Gootenberg et al. 2017; Myhrvold et al. 2018) provides high sensitivity DNA or RNA detection. Specifically, development of the CRISPR-Cas13a protein-RNA system provides attomolar (Gootenberg et al. 2017) to near-zeptomolar (Gootenberg et al. 2018) detection limits, and with multiplexing capability (Myhrvold et al. 2017). The CRISPR-based systems are dependent upon nucleic acid amplification and several enzymes, thus increasing the possibility of false positive or negative signals. Nonetheless, the CRISPR-based systems represent an important advance in amplification-based technologies, and also underscore the specificity achievable in nucleic acid detection based on complementary base-pairing, as opposed to direct sequencing.

RNA Detection Via Hybrid Formation (Northern Analysis)

The original method for RNA detection by hybrid formation is the Northern analysis (Brown 2001). Here, size fractionation by denaturing gel electrophoresis of a purified, formaldehyde-treated RNA preparation is followed by transfer of the RNA to a membrane. Addition of a fluorescent, chemiluminescent, or radiolabeled DNA probe complementary to a sequence in the RNA of interest allows hybrid formation on the membrane. The hybrid (and thus the RNA) is then detected by the signal provided by the label. This method has limited multiplexing capability, and does not easily provide quantitation of RNA levels. The requirement for electrophoresis and the use of labels for detection are impediments for portability and ease of use.

Hybrid Detection by Antibodies

A monoclonal antibody (“S9.6”) was developed that can recognize RNA-DNA hybrids with high affinity (Boguslawski et al. 1986). A single-chain Fv fragment of the S9.6 antibody binds hybrids with a K_{D} of ˜1 nM (Phillips et al. 2013). A fluorescent-labeled version of the S9.6 antibody was used to detect RNA binding to DNA microarrays (Hu 2006). However, the S9.6 antibody retains comparable affinity for double-stranded(ds) RNA (Phillips et al. 2013; Hartono et al. 2018), which is a significant impediment since dsRNA is a common biological structure, and therefore can create false positive signals. Also, the S.96 antibody exhibits an affinity for hybrids that is dependent on the hybrid bp sequence content (König et al. 2017).

Hybrid Detection by a Small Molecule Conjugate

Methidium (a nucleic acid intercalating agent) and neomycin (a naturally occurring antibiotic) are each known to bind RNA-DNA hybrids and dsRNA. Arya and coworkers (Shaw et al. 2008) devised a strategy to achieve substantially higher affinity as well as selectivity for hybrids. The goal was to develop a drug that could block retrovirus infection by targeting the RNA-DNA hybrid structures formed during retroviral replication. Using synthetic chemistry approaches, Arya and coworkers linked methidium to neomycin to provide a methidium-neomycin (MN) conjugate. The MN conjugate exhibits a strong affinity (K_{D}˜0.2 nM) for RNA-DNA hybrids. The MN conjugate also retains an appreciable affinity for dsRNA, with an approximately 20-fold weaker affinity than that for a hybrid. The retention of dsRNA affinity by the MN construct may be a constraining factor in achieving high selectivity in RNA detection via hybrid formation. Also, the requirement for organic synthesis to create the MN construct limits the accessibility of this entity for routine RNA-DNA hybrid detection, and synthetic approaches to further increase hybrid affinity and selectivity remains a challenge.

RNase H

RNase H selectively recognizes and cleaves the RNA strand of RNA-DNA hybrids. To provide a form that can recognize RNA-DNA hybrids without concomitant cleavage, a point mutation was engineered into RNase H that inactivated the catalytic site, without affecting hybrid binding affinity. The purified recombinant mutant protein was used to isolate RNA-DNA hybrids by immunoprecipitation, followed by sequencing the captured nucleic acid, for genomic analyses of chromosome R-loops (Chen et al. 2017).

Atomic Force Microscopy (AFM)

The HBD from human RNase H1 was attached to an AFM tip and used to detect hybrids formed by microRNAs binding to complementary DNA probes affixed to specific locations on an AFM surface (Koo et al. 2016). MicroRNAs also were located in situ, in cells affixed to the AFM surface. In both approaches, the AFM tip-attached HBD induced an adhesive force when it touched a hybrid, but there was no adhesive force in the absence of the microRNA. In a separate study, a self-assembled, water-soluble DNA nanostructure was designed to bind specific RNA sequences at spatially-defined probe sites. RNA binding and hybrid formation created a local topographical change (height increase) that was detected and recorded by a scanning AFM tip (Ke et al. 2008). Both approaches require an atomic force microscope, which is costly, not portable, and requires extensive operator training.

Additional Modifications to the Epiprinter/Epitape

The v.1 epiprinter functions as predicted in high affinity binding and covalent attachment to a model hybrid. In addition, linking three HBD subunits provides a module with even higher (sub-nanomolar) hybrid binding affinity, thus providing the basis for creating a v.2 epiprinter with subnanomolar hybrid binding affinity.

With sequence information on novel RNAs of interest, additional epitapes can be readily produced that have probe sites for additional RNAs, and the multiplexing capability of the technology can readily accommodate additional epitapes. Incorporation of modified oligonucleotides such as locked nucleic acid (LNA) oligos (Hagedorn et al. 2018) in epiptape probe sites can enhance the capacity of the epitape probe site to discriminate between near-identical RNA sequences.

### Example 2: A Click-Reactive, High Affinity RNA-DNA Hybrid Recognition Module (Epiprinter) for Detection and Monitoring of Viral Infection

The emergence and spread of vector-borne viral diseases into new geographic areas are having an increasing impact on human populations. This is underscored by the recent spread of Zika virus (ZIKV) disease into the Caribbean (Mutebi et al. 2018) and the United States (Ryan et al. 2018). The spread of ZIKV and other mosquito-borne viruses, including Dengue and Chikungunya, is spurring the development of methods for the rapid, sensitive and accurate virus detection in resource-limited settings. Rapid identification of viruses in patients in remote settings and the clinic, as well as field surveillance of mosquitoes, can enable earlier deployment of vaccines and treatments and allow more efficient, targeted control of disease vector populations (Nicolini et al., 2017, Journal of Biological Engineering, 11:7; Holmes et al. 2018).

Current virus identification methods primarily involve immunological assays (e.g., ELISA). However, cross-reactivity is a persistent problem (Kam et al. 2015). Nucleic acid detection methods can identify viruses with high sensitivity (Niemz et al. 2011; Maffert et al. 2017), and enzyme-based amplification technologies remain the standard for nucleic acid detection. The recent introduction of CRISPR-based tools (Chen et al. 2018; Myhrvold et al. 2018) is a key advance in amplification-based technologies and underscores the specificity achievable in nucleic acid detection based on complementary base-pairing. However, amplification-based approaches involve multiple steps, involving a polymerase, with the attendant possibility of false negative or false positives (Potapov and Ong, 2017, PLOS ONE, 12: e0169774). Virus detection by nanopore sequencing was demonstrated with the 2015 Ebola virus outbreak in Nigeria (Quick et al. 2016). Here, acquisition of sequence data with the MinION system allowed on-site monitoring of virus spread and genomic change. While nanopore-based sequencing requires several steps, including enzymatic conversion of RNA to cDNA, the study showed the effectiveness of a nanopore-based device in a resource-limited setting (Quick et al. 2016).

Amplification-based nucleic acid detection methods are broadly used, but the need remains for new approaches when reliability, cost, and ease of use are considered (Peeling and Mabey 2010). A method that does not require enzyme or amplification, and provides direct, single-molecule readout (e.g. via nanopore translocation) would enable RNA detection by non-expert personnel in a wide range of settings. Such a point-of-use technology would allow earlier prediction of, and rapid response to disease outbreaks. A closely related issue is that the clinical presentation of acute febrile illness (AFI) can reflect infection (or coinfection) by one or more of a number of viruses (Robinson and Manabe 2017). Here, a multiplexed RNA detection technology would provide rapid identification of the pathogen(s) among the numerous possibilities, and inform the best course of treatment. A multiplexed approach also would improve field surveillance of the disease vectors.

The detection system described here incorporates advances in bio-orthogonal chemistry, DNA self-assembly and nanopore technology for amplification- and enzyme-free RNA detection. FIG. 1 shows the detection scheme. The fundamental basis of detection is the high specificity of pairing of a probe DNA sequence to the complementary target RNA sequence, creating an RNA-DNA heteroduplex (“hybrid”). Hybrids can be specifically recognized by the Hybrid-Binding Domain (HBD) (˜60 a.a., βαββα fold) which is a conserved domain in ribonuclease H1 enzymes. The HBD selectively binds hybrids in a sequence-independent manner with sub-micromolar affinity (Nowotny et al., 2008, The EMBO Journal, 27: 1172-1181; Jongruja et al., 2010, FEBS Journal, 277: 4474-4489). While specific antibodies can recognize hybrids(Phillips et al. 2013), they are less selective (Hartono et al. 2018) and their size limits their ease of use. The HBD will be further elaborated to provide a high-affinity hybrid recognition module (“epiprinter”) that is enabled for “click” chemistry. Here, the spontaneous covalent reaction of an alkyl azide with an alkynyl group, either by a copper (Cu[I])-dependent (Presolski et al. 2011) or Cu[I]-independent (Jawalekar et al. 2013) pathway, creates a stable triazole. Click chemistry is broadly applied for efficient conjugation of biomolecules, and its bio-orthogonality minimizes off-target reactions (Gierlich et al. 2006). The epiprinter will be used in conjunction with a custom DNA nanostructure (“epitape”).

Further improvement in covalent attachment may be gained by using a p-phenylazido (azF) group (Marth et al. 2017). Here, p-azido-L-phenylalanine is incorporated in vivo into the DHBD or THBD linkers, in place of a cysteine residue. azF-DHBD (or THBD) production will be achieved in an E. coli host grown in azF-containing growth medium, that expresses a modified aminoacyl-tRNA synthetase and cognate tRNA that together direct incorporation of azF at the engineered stop codon (Marth et al. 2017). Affinity-purified azF-DHBD/THBD will be combined with the BCN-DNA hybrid and reactivity assessed as described above. This establishes an optimized proto-epiprinter, as (for example) an azF-substituted THBD with optimized linkers.

The DHBD exhibits a >10-fold enhancement in hybrid binding compared to the HBD (FIG. 2C), and the THBD exhibits a >10-fold increase. Optimized linker lengths should confer additional affinity. The Cu-independent SPAAC chemistry supports efficient coupling and obviates the use of Cu, thus simplifying the detection protocol. The use of azF-containing THBD should provide a further enhancement of reactivity (Dommerholt et al. 2014).

The detection system uses a custom DNA nanostructure (epitape) to covalently record RNA binding and to generate a nanopore signal. DNA nanostructures have been developed that bind specific RNAs in a sequence-specific manner, with detection achieved by atomic force microscopy (AFM) (Ke et al. 2008). Elongated DNA nanostructures (“carriers”) also have been developed that can site-specifically bind multiple target ligands (Bell and Keyser 2015; Bell and Keyser, 2016, Nature Nanotechnology, 11: 645-651). Here, the noncovalent binding events are detected by passage of the DNA carrier through a quartz nanopore, generating recordable, unique current signatures.

The proto-epitape (FIG. 3) has three key elements: the carrier, two probe sites, and barcode. The carrier consists of the circular ssDNA of M13 phage (˜7.2 kb; from NEB), folded by ˜30-40 nt DNAs (“staples”) to provide a stable, linear structure (˜1.1 μm). Self-assembly follows an established protocol (Rothemund 2006) in which the staples bind to the scaffold to create the carrier as an elongated 2-helix bundle (FIG. 3) with thymine(T) end overhangs to prevent aggregation. The two helices (˜3600 bp each) are joined by periodic crossovers to create a flat, linear “tape” (Dietz et al. 2009). The estimated (Landau and Lifshitz, 2013) persistence length of ˜80 nm (˜300 bp), which is greater than that of double-helical DNA (Kong et al., 2016, Nano Letters, 16: 3557-3562; Plesa et al. 2015) reduces signal-to-noise and false positive signals by minimizing knot formation.

Proto-epitape design uses cadnano software (Douglas et al. 2009), which minimizes design errors and guides programmable modifications. Based on the Ke et al. (2008) study, each probe site will be comprised of two ˜10-nt staple extensions, in close proximity, that protrude from the carrier surface (FIG. 3, bottom right). Each probe site strand pair is complementary to a ˜20 nt RNA sequence. Each strand carries an alkynyl (or BCN) group (FIG. 3), permitting attachment of two epiprinters. Barcodes consist of hairpin structures (“dumbbells”) (FIG. 3) (Bell and Keyser, 2016, Nature Nanotechnology, 11: 645-651), positioned near one end of the proto-epitape. These provide digital identification of the proto-epitape, and establish proto-epitape orientation during nanopore translocation.

The proto-epitape is assembled and purified, and the structure verified by AFM and gel electrophoresis. Proto-epitape yield and quality is assessed as a function of Mg and salt (LiCl) concentrations (Martin and Dietz 2012) and thermal protocol alteration (Sobczak et al. 2012). RNA binding to the proto-epitape is evaluated by gel electrophoresis. A 20 nt RNA from a model RNA virus (phage MS2) is used, as well as the full-length, 3569-nt MS2 ssRNA chromosome prepared by in vitro transcription (Miller et al. 2002). The second probe site can target a 20 nt RNA sequence unique to the ZIKV genome (Cunha et al. 2016). RNAs carry fluorescent tags and the proto-epitape carries a non-overlapping fluorophore. RNAs are combined with the proto-epitape and gel electrophoresis with fluorimetric imaging is used to assess RNA binding. Control experiments use non-complementary RNAs or a proto-epitape lacking probe site sequences.

Proto-epitape binding of full-length MS2 RNA is assessed by gel shift assays and by proto-epiprinter modification. The proto-epitape with bound RNA is combined with the proto-epiprinter and attachment chemistry performed based on alkynyl group type. Proto-epiprinter attachment is verified by AFM or by gel electrophoresis and imaging, using fluorescent-labeled epiprinter. Control reactions omit RNA, or use proto-epitapes lacking alkynyl groups.

Analysis of epiprinter attachment involves detection of current change caused by proto-epitape translocation across a quartz nanopore of ˜10-20 nm internal diameter (Bell et al., 2013, Lab on a Chip, 13:1859). Quartz nanopores are readily fabricated and exhibit minimal high frequency noise (Bell et al., 2013, Lab on a Chip, 13:1859; Steinbock et al., 2013, ACS Nano, 7:11255-11262). A ˜4 M LiCl carrier solution should provide a translocation rate optimal for signal analysis (Kowalczyk et al., 2012, Nano Letters, 12:1038-1044). Ion currents are measured by patch clamping and recorded at a 100 kHz rate. Data analysis is performed using MATLAB. The voltage is identified that provides a consistent epitape translocation signal (Raveendran et al. 2018; Bell and Keyser, 2016, Nature Nanotechnology, 11: 645-651), as well as the optimal ion current peak width (dwell time) and amplitude during translocation. The minimal spacing is determined between the probe sites that provides distinct ion current peaks for each site. In turn, this is used to establish the optimal distance between the probe and barcode elements in proto-epitape. To assess nanopore ability to discriminate modified from unmodified proto-epitapes, analyses compare (i) proto-epitape, (ii) proto-epitape with bound RNA, and (iii) proto-epitape with attached epiprinter. To determine whether bound RNA survives the procedure, the recovered proto-epitape is subject to DNase treatment and RT-PCR used to determine RNA integrity and yield.

The proto-epitape is assembled in high yield (>95%) and binds RNA as predicted due to the relatively simple, established structure, and by employing motifs shown elsewhere to function as probe sites or barcodes. The Cu-independent click chemistry is robust and simplifies the detection approach. The demonstration of quartz nanopore detection of ligands bound to a double-helical DNA (Bell and Keyser, 2016, Nature Nanotechnology, 11: 645-651) demonstrates that one or two ˜17 kDA epiprinters attached at a probe site provide a steric bulk sufficient to cause a detectable current change.

Incorporated “barcode” structures allowed accurate identification of the coupled ligands (Bell and Keyser, 2016, Nature Nanotechnology, 11: 645-651). However, this approach is sensitive to disruption of the noncovalent binding interactions, thus compromising robust detection. The proposed epitape (FIG. 1) will be an elongated, stable DNA nanostructure with RNA-binding probe sites carrying click-reactive alkynyl groups. The probe sites will selectively bind the complementary target RNA, forming a hybrid that is recognized by an added epiprinter, which then covalently attaches to the DNA strand by click chemistry. Ion current change during solid-state (quartz) nanopore translocation of the epitape with the covalently attached epiprinter generates the readout signal of the initial RNA binding event (FIG. 1).

In summary, the development of a functional prototype epiprinter-epitape will guide the construction of epitapes with greater multiplexing capability for high-sensitivity RNA detection. The significance of this achievement is the foundation for an amplification-free, nanopore-based RNA detection technology for rapid identification of disease agents in patients or in insect vectors, deployable in a broad range of resource-limited settings. Effective use of this technology in turn will serve to inform the best treatment strategy and provide early identification of the location and type of disease-carrying vector populations, allowing intervention and control strategies.

A Click-Reactive, High Affinity RNA-DNA Hybrid Recognition Module (Epiprinter).

The hybrid-binding domain (HBD) is a conserved protein fold that selectively recognizes RNA-DNA hybrids. The HBD is converted to dimeric and trimeric form, with linker lengths optimized for binding affinity, as informed by molecular modeling, and hybrid binding affinities are determined by gel shift and SPR assays. Alkyl azide group incorporation provides a prototype epiprinter with click capability. Using model hybrids with alkynyl-DNA backbones, optimal in vitro epiprinter function is established for both Cu-dependent and Cu-independent click chemistries. The effect of hybrid length, base mismatches, and alkynyl group type and position on epiprinter binding is determined.

Custom DNA Nanostructure (Epitape) for Epiprinter-Dependent RNA Detection by Nanopore Readout.

A prototype epitape was assembled from a DNA scaffold and oligonucleotide “staples.” The proto-epitape contains barcode structures for nanopore readout. Probe sites are created by selected staples with sequence extensions that are accessible and complementary to the RNA targets, and carrying click-reactive alkynyl groups. Epiprinter attachment to proto-epitape probe sites bound to an RNA is verified using an epiprinter with fluorescent label. The change in ion current in response to nanopore transit of epiprinter-modified proto-epitape will be compared to unmodified proto-epiprinter. The structural integrity of the bound RNA will be evaluated by RT-PCR.

These experiments provide the basis for nanopore detection of multiple RNAs using custom DNA nanostructures. Specifically, the prototype system can guide the development of more complex epitapes, or epitape sets, that can detect multiple viral (or any) RNAs at low concentrations in complex samples. Since the detection method is designed to be nondestructive to the RNA, it also allows further characterization of the bound RNA, as needed.

### Example 3: A Fluorescent-Labeled Epiprinter Precursor, THBD-AF647, can Detect DNA-RNA Hybrid Structures in Mammalian Cells

The experiments presented here demonstrate the ability of the THBD, modified to contain a fluorescent label, to detect DNA-RNA hybrid structures in mammalian cells.

The prototype epiprinter (THBD), carrying a fluorescent reporter label, offers several advantages over the S9.6 antibody, as its smaller size (21 kDa vs 150 kDa) enables more efficient diffusion into dense and nanoconfined intracellular sites, thus locating and binding R-loops, and other DNA-RNA hybrid structures, with fewer steric constraints. The THBD also has greater specificity for DNA-RNA hybrids (compared to dsRNA), thus minimizing false positive signals, and also is insensitive to base-pair sequence, thus minimizing bias in R-loop/hybrid recognition.

The HBD binds hybrids with an affinity that is 20-fold greater than its affinity for dsRNA (Nowotny et al., 2008, The EMBO Journal 27:1172-1181), while this difference is only 5-fold for the S9.6 antibody (Phillips et al., 2013, Journal of Molecular Recognition, 26:376-381). The unique structural design of the THBD, containing three copies of the HBD, also would provide substantially higher affinity than the single HBD. Finally, the ease of preparation of the THBD, along with its stability and facile modification including fluorescent label attachment, provides a user-friendly, cost-effective alternative approach to map and characterize R-loops and other intracellular DNA-RNA hybrids.

These results demonstrate that a fluorescent-labeled THBD is able to detect R-loops within mammalian cells. Thus, the prototype epiprinter can detect DNA-RNA hybrids in complex intracellular environments, which supports the prospects that epiprinter detection of RNA should be attainable in a variety of other complex environments presented by diverse biological samples. The fluorescent-labeled THBD may also be an attractive reagent that could be provided as part of a kit for in situ fluorescence detection of DNA-RNA hybrids, and also for affinity purification of cellular DNA-RNA hybrids for sequencing and further analysis.

The Materials and Methods used are now described

Protein Labeling.

The Triple HBD (THBD) DNA construct was assembled into the pET-15b DNA plasmid vector, domesticated for the BsaI restriction enzyme, using Golden Gate Assembly cloning kit (NEB, USA), and following the supplier's protocol. The sequence was verified by Sanger sequencing. The His-tagged THBD protein was expressed in BL21(DE3) E. coli cells and purified by affinity purification. The His-tag was removed by Thrombin reaction directly on the affinity column. A version of the THBD, which bears a single cysteine, was chemically modified using the Alexa Fluor™ 647 C2 Maleimide kit (Thermo, USA). The Alexa Fluor 647-labeled THBD protein was purified from the maleimide-sulfhydryl cross-linking reaction mixture using size exclusion chromatography (Superdex 75 Increase 10/300 GL Column) on an AKTA FPLC system (GE Healthcare, USA). The mouse anti-DNA-RNA hybrid monoclonal S9.6 antibody (Millipore-Sigma) was labeled using the Cy3 Ab Labeling Kit (GE Healthcare, USA) that uses NHS ester conjugation to protein amino groups (Lysine side chains). The Cy3-labeled S9.6 antibody was then purified using Amicon spin filters (Millipore-Sigma) and the dye:protein ratio of 5:1 was determined with absorbance measurement by Nanodrop spectroscopy.

HeLa Cell Growth and Fluorescence Imaging.

HeLa cells were cultured on coverslips in a 6-well plate. After one day they were washed with PBS, permeabilized for 5 min. with 0.1% Triton X-100 solution, then incubated with blocking solution (3% BSA in PBS). The cells then were stained by incubation with 0.2 μM THBD-A647 (1:1 dye:protein ratio) for one hour, then with 0.2 μM S9.6 Ab-Cy3 (5:1 dye:protein ratio) for 1 hour, then 1× Hoechst dye solution for 5 min. The cells were washed with PBS and covered with a coverslip using a mounting medium. Images were captured with a confocal fluorescence microscope using a 30× objective. The Cy3 excitation fraction at 561 nm was 0.78, while for Alexa Fluor 647 the excitation fraction at 640 nm was 0.83.

The results of the experiments are now described.

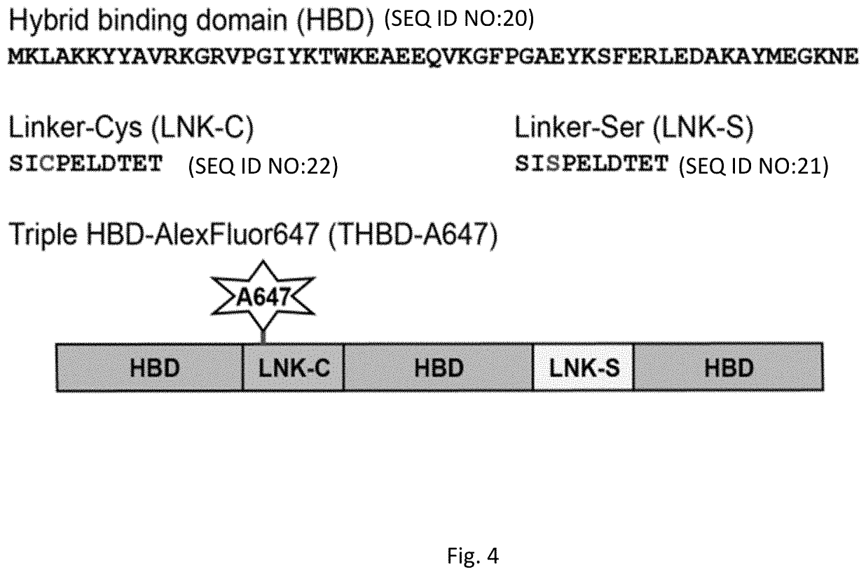

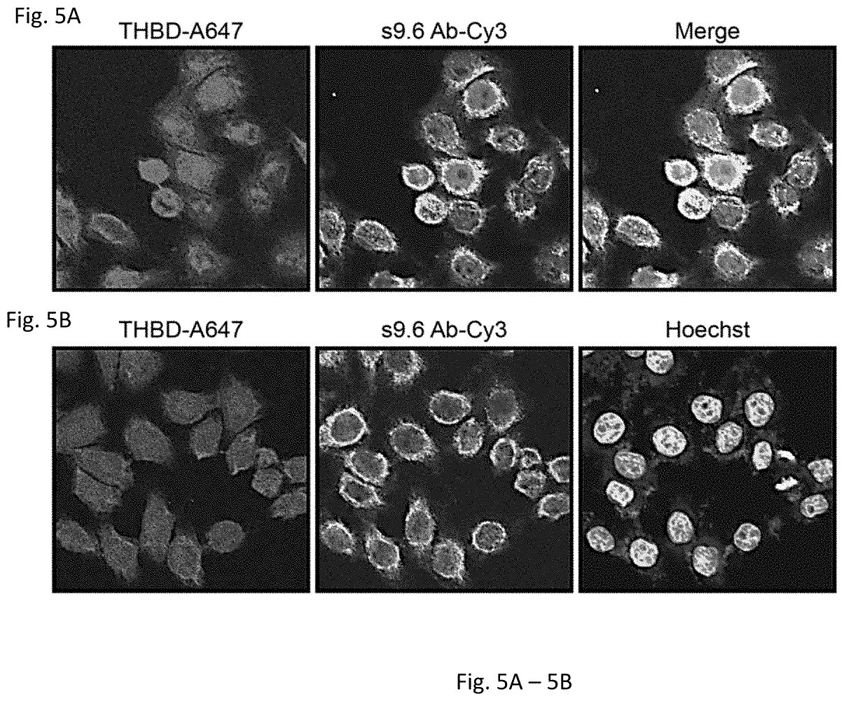

Mammalian HeLa cells have been used in other studies to analyze the function and dynamics of R-loops, using the S9.6 antibody as probe (Bhatia et al. 2014; Cristini et al. 2018). The purified THBD was modified using the Alexa Fluor™ 647 C2 Maleimide kit (Thermo, USA), such that the THBD carried a single AF647 molecule on a cysteine residue in the first linker peptide (FIG. 4). The purified THBD-AF647 was introduced into fixed HeLa cells, and the fluorescent signal captured by confocal microscopy. The results (FIG. 5) show that the THBD (left panels in FIG. 5A and FIG. 5B) can stain the nucleus. In this regard, the right-most panel in FIG. 5B (labeled “Hoechst”) shows the nuclear staining provided by the DNA-staining Hoechst dye. This confirms that the THBD-AF647 fluorescence is localized to the nucleus.

A comparison experiment, performed using the S9.6 antibody labeled with Cy3 fluorescent dye (S9.6 Ab-Cy3), is shown in the middle panels in FIG. 5A and FIG. 5B. This experiment shows, in agreement with prior studies, that the S9.6 Ab-Cy3 antibody fluorescence concentrates in the nucleus. The right-most panel in FIG. 5A (labeled “Merge”) shows the nuclear co-localization of fluorescence of THBD-AF647 and S9.6Ab-Cys. In that panel, the red spots in the nuclei reveal that the THBD-AF647 can preferentially localize to the nucleoli, which are sub-nuclear compartments that are the site of extensive gene transcription and R-Loop formation.

### Example 4: Determination of HBD, DHBD and THBD Binding Affinities for a Model RNA-DNA Hybrid

The ability of the epiprinter to bind target hybrid structures with high affinity and specificity is essential to its intended use in the sensitive and accurate detection of RNA. Surface Plasmon Resonance (SPR) is a powerful method for characterizing biomolecular interactions and binding affinities. SPR can provide essential kinetic data (on- and -off rates), thermodynamic data (K_{D} values) and stoichiometries of the components that form a complex, over a broad range of experimental conditions. SPR has been successfully used to characterize the binding of proteins to nucleic acids, including the S9.6 antibody to DNA-RNA hybrids (Phillips et al. 2013; Binz et al. 2004).

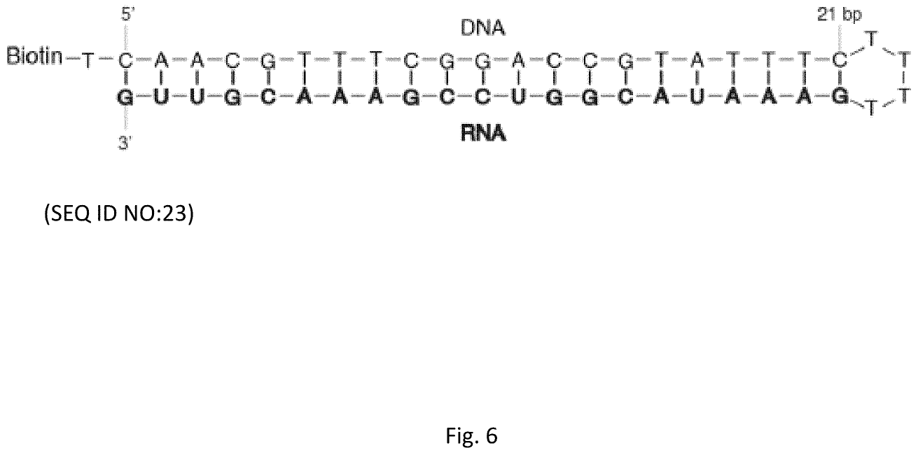

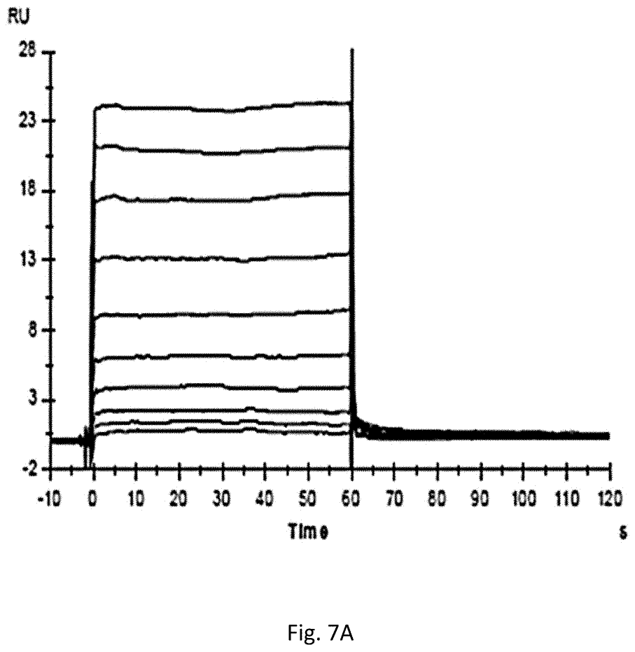

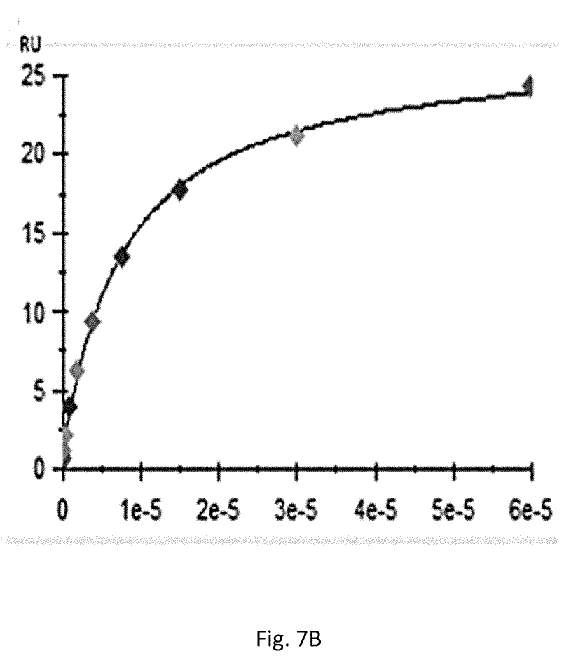

The binding of the purified HBD, DHBD and THBD polypeptides to a 21 bp DNA-RNA hybrid (in the form of a hairpin; (FIG. 6) was determined using a Biacore T200 SPR instrument. The hybrid carries a 5′-biotin moiety and was immobilized on a chip surface that was modified to contain Neutravidin. The protein (HBD, DHBD, THBD) in solution was passed over the chip surface, and protein binding to the hybrid was determined by measuring change in index of refraction, corresponding to the increase in mass upon complex formation. The output signal is reported as response units (RU). The DNA-RNA hybrid was immobilized upon the chip surface at low density (-10 RU). Next, the solution of the protein of interest was injected (over a 60 sec time period), providing an output signal with three phases: initial binding (association), steady-state (complex), and complex dissociation (protein release). The binding assay was repeated using a series of protein concentrations, generating a collection of curves (see FIGS. 7A, 8A, 9A). Fitting of the kinetic and steady-state data, using a two-state binding model, enabled determination of the kinetic and affinity parameters.

Materials and Methods

SPR analyses were performed using a Biacore™ T200 instrument (Wistar Institute Proteomics Facility). The analysis buffer was 50 mM Sodium Phosphate (pH 7.4), 100 mM NaCl, 1 mM EDTA, and 0.05% (v/v) Tween-20. A CM4 sensor chip was functionalized with biotinylated RNA-DNA hybrid hairpin (Biotin-RD), following a previously published protocol (Vaidyanathan et al. Nat Prot), with some adaptation. Briefly, Neutravidin (Pierce) was diluted to 0.1 mg/mL in 10 mM Sodium Acetate (pH 5), and using the Amine Coupling Kit (GE Healthcare, Life Sciences), was injected at 20 μL/min, using reiterative cycles of injection, until the Neutravidin immobilization levels reached 1000 RU. The DNA-RNA hybrid hairpin (FIG. 6) was dissolved in 10 mM Tris (pH 8), 50 mM NaCl, at 100 nM and 1 μM concentrations. The solutions were injected at 2 μL/min, using reiterative cycles of injection, until the Biotin-hybrid reached immobilization levels of ˜10 RU (Flow cell 2). Protein serial dilutions were prepared in using the buffer described above, and were injected at 50 μL/min. All experiments were performed at 25° C., and in triplicate. Data transformation of the primary sensograms, and overlay plots were prepared using BIAevaluation 3.2 software (GE Healthcare, USA).

Binding of HBD, DHBD and THBD to the DNA-RNA Hybrid

FIG. 7A shows that HBD can bind to the immobilized hybrid, providing a progressively greater output signal in an HBD concentration-dependent fashion. The binding curves revealed very fast association (kₒₙ) and dissociation (k_{off}) rates, that rapidly progressed to equilibrium even at low protein concentrations. In fact, the rates of HBD binding and release were too rapid to allow accurate fitting of the curves to determine the on-rate (kₒₙ), the off-rate (k_{off}), and the K_{D} by kinetic analysis. However, the steady-state data allowed determination of the affinity of the HBD for the hybrid. FIG. 7B shows the RU values at equilibrium as a function of HBD concentration. Fitting the data to a two-state model provided an equilibrium dissociation constant (K_{D}) for the HBD of 7.97 (±0.18) μM.

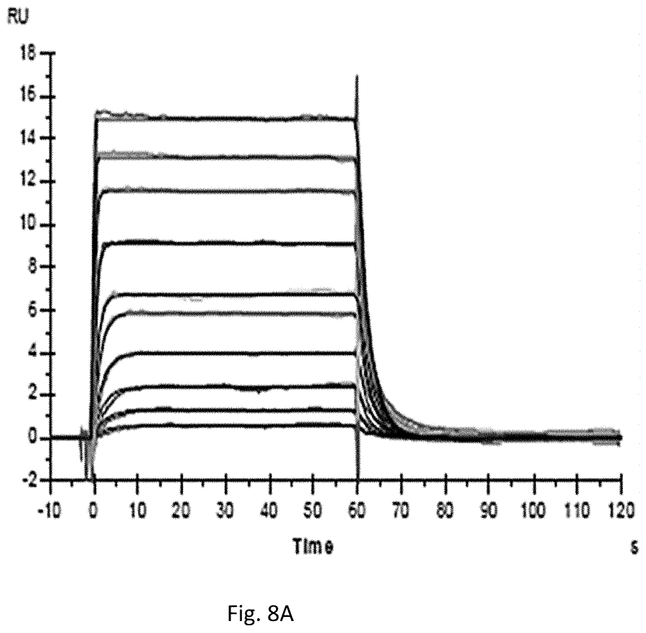

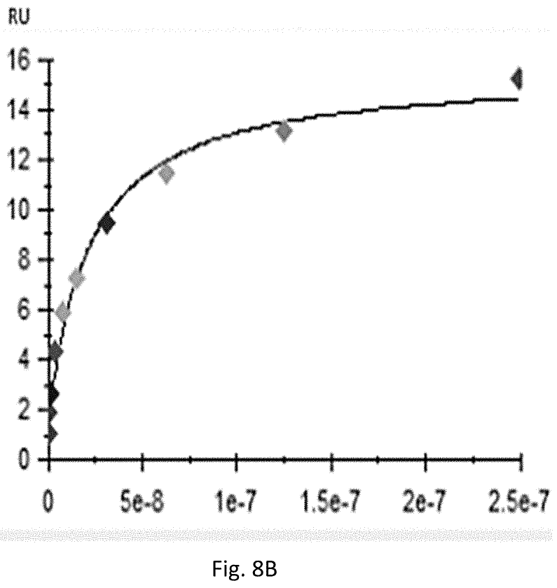

The DHBD affinity for the DNA-RNA hybrid was determined. The results (FIG. 8A) show that DHBD shows a quantitatively different binding behavior compared to the HBD, in that the association and dissociation rate constants are slower. This allowed determination of the kₒₙ and k_{off} values, and therefore also the K_{D} value (Table 1). Comparing the steady-state values, the DHBD affinity (K_{D} of ˜20 nM) is 400-fold higher than the HBD affinity for hybrid, this is also true considering either the value obtained with the steady-state model, or the kinetic model.

TABLE 1
Binding affinities of the 21 bp DNA-RNA hybrid for
the HBD, DHBD andT HBD as determined by SPR. The
averages and standard deviations for all values are
derived from three experimental replicates.

| | | | K_{D} | K_{D} |
|---|---|---|---|---|
| | | | kinetic | steady |
| | kₒₙ | k_{off} | model | state model |
| Protein | (×10⁶ M⁻¹s⁻¹) | (×10⁻³ s⁻¹) | (×10⁻⁹M) | (×10⁻⁹M) |
| HBD | ND | ND | ND | 7970 ± 180 |
| DHBD | 31 ± 6 | 355 ± 40 | 12 ± 2 | 20 ± 1 |
| THBD | 126 ± 19 | 25 ± 8 | 0.19 ± 0.02 | 0.380 ± 0.006 |

ND, non-determinable data.

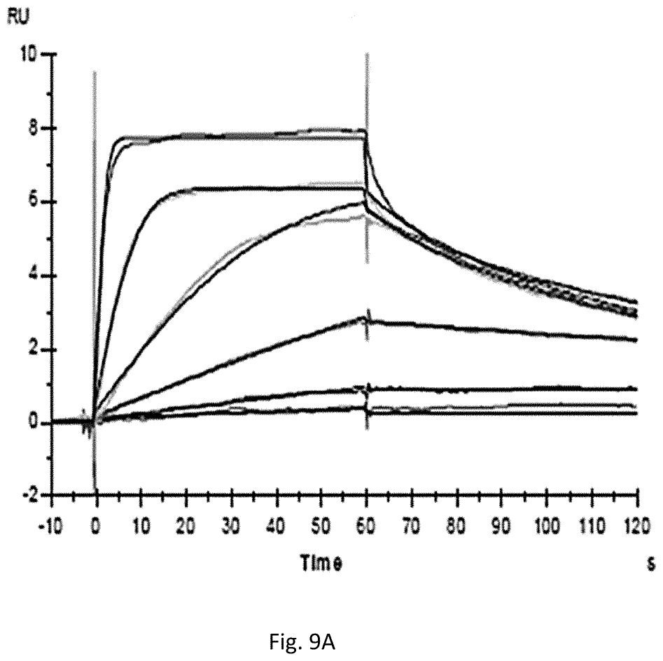

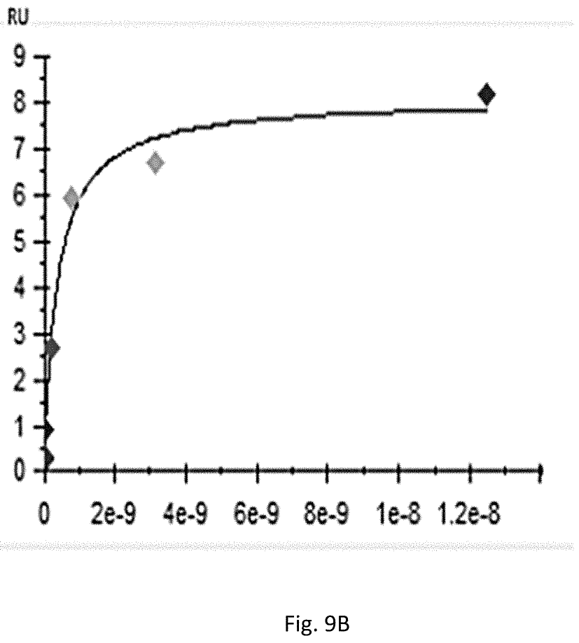

The THBD exhibits a kinetic profile dramatically different from DHBD and HBD. The sensorgram (FIG. 9A), showing the concentration-dependent response of THBD binding to the hybrid, reveals that the THBD can bind the hybrid at concentrations as low as 12 pM. The on rate, very fast, remains similar to HBD and DHBD, but the key change occurs with the off rate, that decreases by ˜14-fold compared to that of the DHBD (Table 1). The slower off-rate results in an increased residence time on the hybrid. Thus, the THBD retains fast binding ability, but remains bound for a significantly longer time. This is reflected into a high affinity for DNA-RNA hybrid; the K_{D} values range in picomolar affinity, between 0.2 and 0.4 nM (see Table 1). The retained fast on-rate, and retarded off-rate, are the desired qualities for an epiprinter to function in RNA detection.

Stoichiometry of Binding of HBD, DHBD, and THBD to the 21 bp DNA-RNA Hybrid Hairpin

The maximum response (Rmax) reflects the maximum quantity of protein that can be bound by the immobilized DNA-RNA hybrid. The Rmax value can be obtained by extrapolating the response from the injection of high concentrations of protein that saturate the surface. The theoretical Rmax, calculated using Equation 1, depends upon the immobilization level (RL), the molecular weight of the 21 bp DNA-RNA hybrid (15.7 kDa), the molecular weight of the protein, and the binding stoichiometry (Sm).

$${R}_{max} = {R}_{L} \times \frac{{MW}_{Protein}}{{MW}_{Hybrid}} \times {S}_{m} . \qquad Equation 1$$

A comparison of the theoretical and experimental Rmax values can provide information on the binding competence of the surface-attached molecule, and the binding stoichiometry of the interaction under study. The theoretical and experimental Rmax values (Table 2) were determined by assuming a 1:1 binding stoichiometry. The experimental stoichiometry of binding was estimated between the 21 bp hybrid hairpin and each of the three proteins, using Equation 2, as derived from Equation 1.

$${S}_{m} = {R}_{\vert max} \frac{{R}_{max}}{{R}_{L}} \times \frac{{MW}_{Hybrid}}{{MW}_{Protein}} . \qquad Equation 2$$

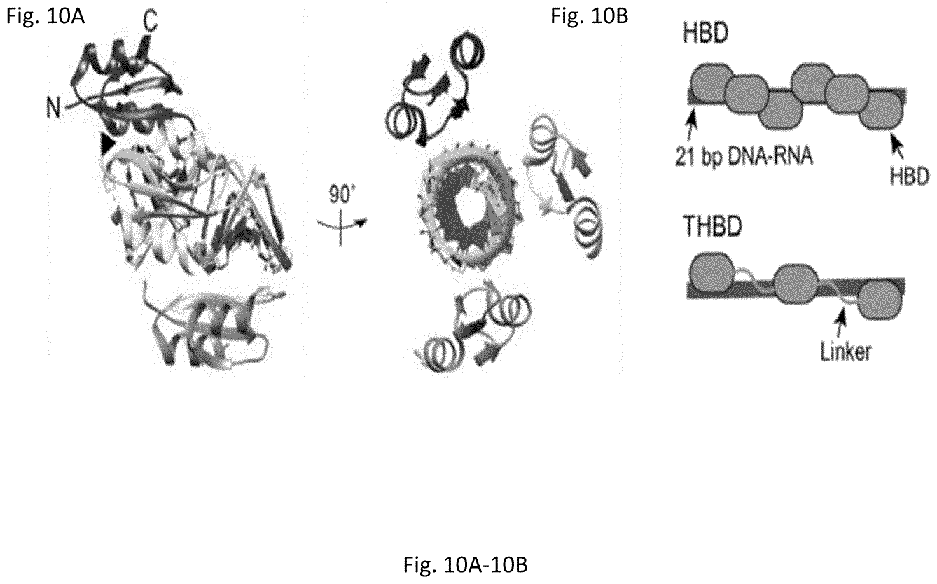

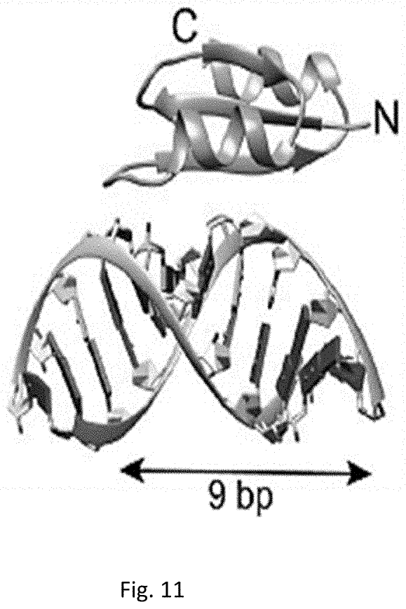

The calculation indicates that six copies of HBD can bind the 21 bp DNA-RNA hybrid. In this regard Nowotny and co-workers (Nowotny et al., 2008, The EMBO Journal, 27: 1172-1181) reported crystallographic data showing that three copies of human HBD (having a similar size [49 amino acids] as the HBD (Thermotoga maritima) studied here) binds a 12 bp DNA-RNA hybrid (FIG. 10A). An individual HBD directly interacts with 5 bp of hybrid, but the entire protein structure spans 9 bp (Nowotny et al., 2008, The EMBO Journal, 27: 1172-1181). Based on this comparison, a 21 bp hybrid is expected to maximally bind no fewer than five copies of HBD. These results are consistent with the crystallographic data. In addition, the stoichiometries in Table 2 are non-integer numbers as they derive from values that are experimental (i.e. Rmax). The experimental values reflect the effective (functionally competent) concentrations of both the immobilized molecule and the injected protein. The biomolecule effective concentrations may be a fraction of the measured concentration, as the effective concentration depends on biomolecule purify, stability, or biases in quantification. For instance, for an interaction with 1:1 stoichiometry, the experimental value may be 0.49 as the effective fraction of immobilized molecules is 0.7 and the effective fraction of protein is 0.7. Given these considerations, we assume that the binding stoichiometry for DHBD and THBD are ˜2 and ˜1, respectively. These values are consistent with the larger sizes of the DHBD and THBD, which means that fewer copies of the proteins can bind the 21 bp hybrid. Thus, the maximal level of hybrid occupancy by the DHBD and the THBD is less than that measured for the single HBD. In this regard, two DHBDs are equivalent to four HBDs; and one THBD is equivalent to three HBDs. FIG. 10B provides a model of how the presence of the 10-amino acid linker in the DHBD and THBD could increase the required space for binding, in order not to interfere with the optimal binding of each HBD subunit. Thus, the two linkers in THBD would reduce the maximal packaging density. Therefore, the THBD requires at least ˜21 bp of a hybrid structure in order to provide maximally effective binding.

TABLE 2
Theoretical Rmax values were computed using Equation 1,
assuming a 1:1 binding stoichiometry. The experimental Rmax
values were computed from the steady-state data using the
global data analysis tool available in BiaEvaluation 3.2 software,
and is the average of three technical replicates. The
Experimental Stoichiometry was computed using Equation 2.

| | | Rmax* | Rmax* | |
|---|---|---|---|---|
| Protein | Protein | Theoretical | Experimental | Stoichiometry* |
| Name | MW | (RU) | (RU) | Experimental |
| HBD | 6730 | 4.3 | 26.23 ± 0.35 | 6.11 |
| DHBD | 14233 | 9.1 | 14.27 ± 0.07 | 1.58 |
| THBD | 21736 | 13.8 | 7.90 + 0.06 | 0.57 |

(* values used for computing: R_{L} = 10 RU).

Modeling of the Thermotoga maritima HBD Interaction with the DNA-RNA Hybrid

The three-dimensional structure of the Thermotoga maritima RNase H1 HBD (Tma-HBD) might provide valuable insight in understanding how the HBD and the hybrid interact. But such a structure has not been experimentally determined yet. Thus, a bioinformatical approach was used to obtain a theoretical structural model of the Tma-HBD using comparative homology modeling. The structure of the human orthologue HBD (h-HBD), PDB ID: 3BSU, was used as template, obtained from X-ray diffraction (Nowotny et al., 2008, The EMBO Journal, 27: 1172-1181). Using blastp alignment tool, the sequences of Tma-HBD (accession no. AHD18801.1) and h-HBD were determined to be 43% identical. The model was generated by the Swiss-Model web server (Waterhouse et al. 2018).

### Example 5: Cloning of the HBD, DHBD and THBD Genes in the PET15B DNA Plasmid

HBD Gene Cloning

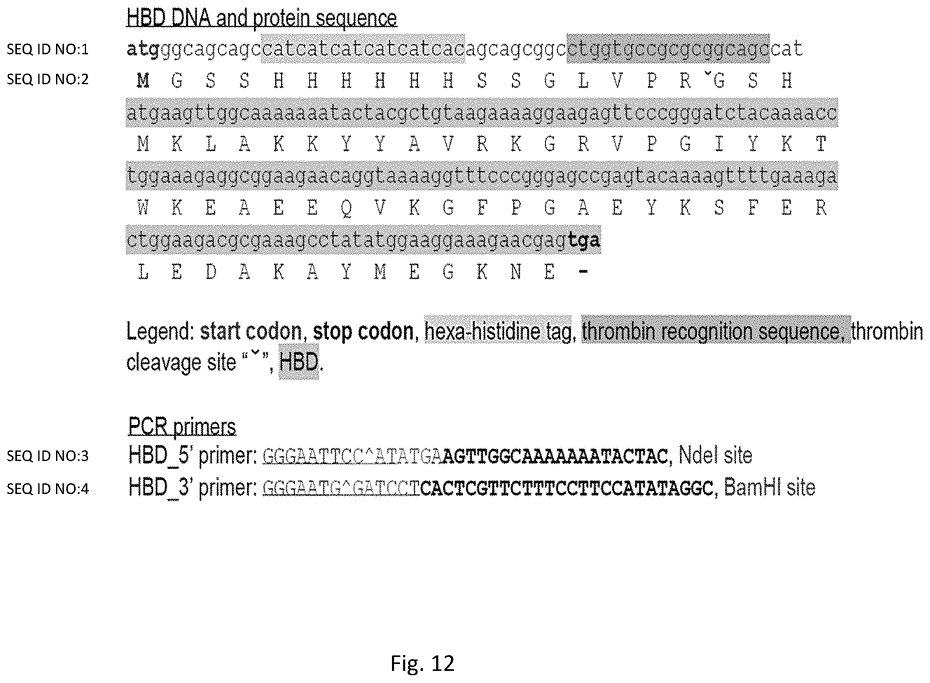

The Thermotoga maritima (Tma) ribonuclease HI gene (NIH GenBank Accession No. AHD18801) encodes a protein of 223 aa, which is comprised of an N-terminal Hybrid Binding Domain (HBD), followed by a 10aa flexible linker and a C-terminal catalytic domain. The HBD DNA sequence was amplified by PCR from a sample of Tma genomic DNA. The PCR primers provided NdeI and BamHI restriction enzyme recognition sequences that flanked the amplified HBD coding sequence. The purified DNA was digested with BamHI and NdeI and ligated into NdeI-, BamH1-cleaved pET-15b plasmid, in the proper orientation for protein expression, and also encoding an N-terminal hexahistidine affinity tag (FIG. 12). The resulting recombinant HBD protein carried a hexahistidine tag, a thrombin protease cleavage site, and the HBD sequence (FIG. 12). The DNA sequence was verified by dideoxy sequencing.

HBD protein physical parameters: 75 aa, 8612 Da molecular mass; after thrombin cleavage: 58 aa, 6730 Da molecular mass. The thrombin-cleaved, recombinant HBD contains the three amino acid GSH sequence at the N-terminus, as a remnant feature of the thrombin cleavage site.

DHBD Gene Cloning

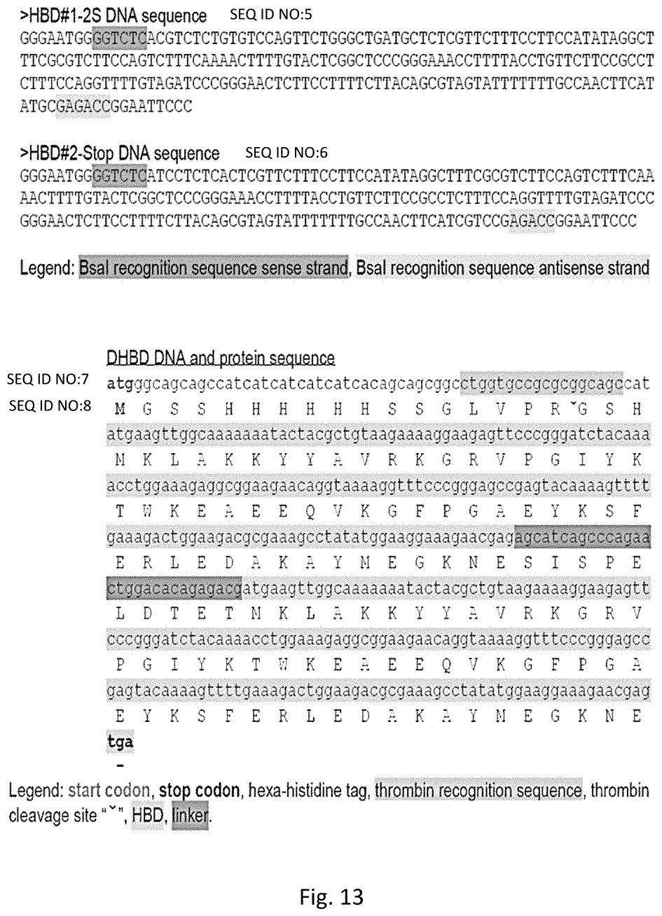

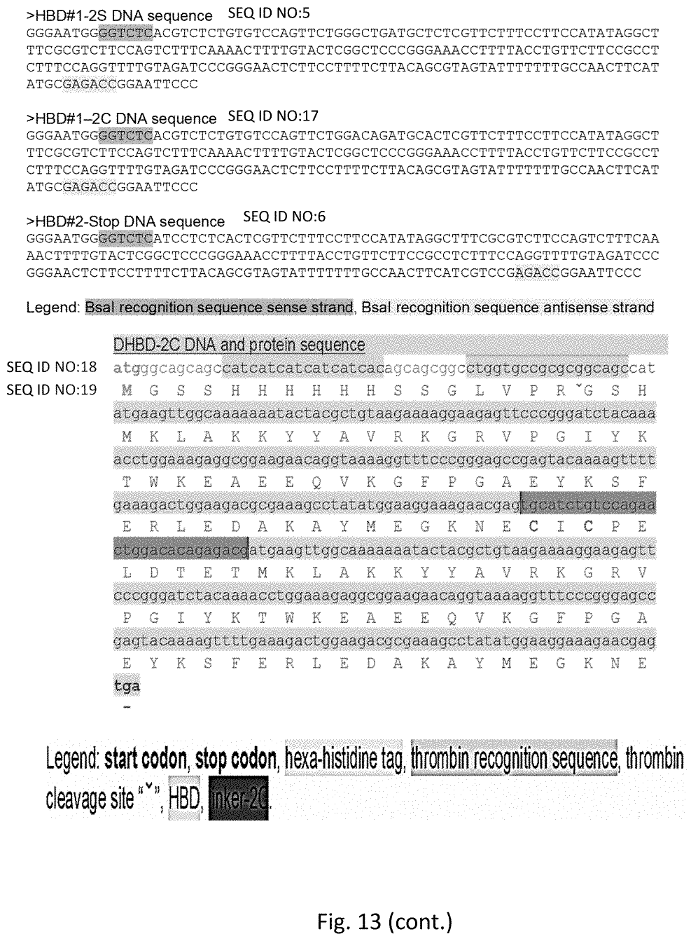

The Golden Gate Assembly cloning strategy (Engler et al., 2008, PLoS ONE, 3:e3647) and BsaI restriction enzyme were used to create the pET15b-DHBD construct. The designed DHBD gene was cloned into an in-lab constructed version of the pET15b vector, domesticated for use with BsaI enzyme. The DHBD gene sequence contained two copies of the HBD DNA sequence of Tma ribonuclease HI. The synthetic gene construct includes, from N- to C-terminus, an HBD, a linker region (from the Tma ribonuclease HI), and an HBD. To implement Golden Gate assembly cloning, the two DHBD coding sequences were presented in two separate DNA fragments: HBD #1-25 and HBD #2-Stop (FIG. 13). Each DNA construct was flanked on both extremities by a BsaI recognition sequence. Reaction of each construct with BsaI provided overhangs that directed the assembly of the two fragments into the pET15b vector, in the proper order and orientation. The HBD #1-2S and HBD #2-Stop DNA constructs were purchased as synthetic dsDNAs, using gBlock technology from IDT, Inc. (Coralville, Iowa, USA). The Golden Gate Assembly protocol (Engler et al., 2008, PLoS ONE, 3:e3647) involved a “one pot” reaction, in which BsaI digestion (New England Biolabs, USA) and ligation using T4 DNA ligase (New England Biolabs, USA) occur simultaneously. The reaction product then was used to transform, by electroporation, ElectroMAX DH10B competent E. coli cells (Life Technologies) that allowed amplification and isolation of the pET15b-DHBD recombinant plasmid. The DNA sequence was verified by dideoxy sequencing (Genewiz).

DHBD protein physical parameters: 140 aa, 16115 Da; after thrombin cleavage site, 123 aa, 14233 Da.

THBD and THBD-1C Gene Cloning.

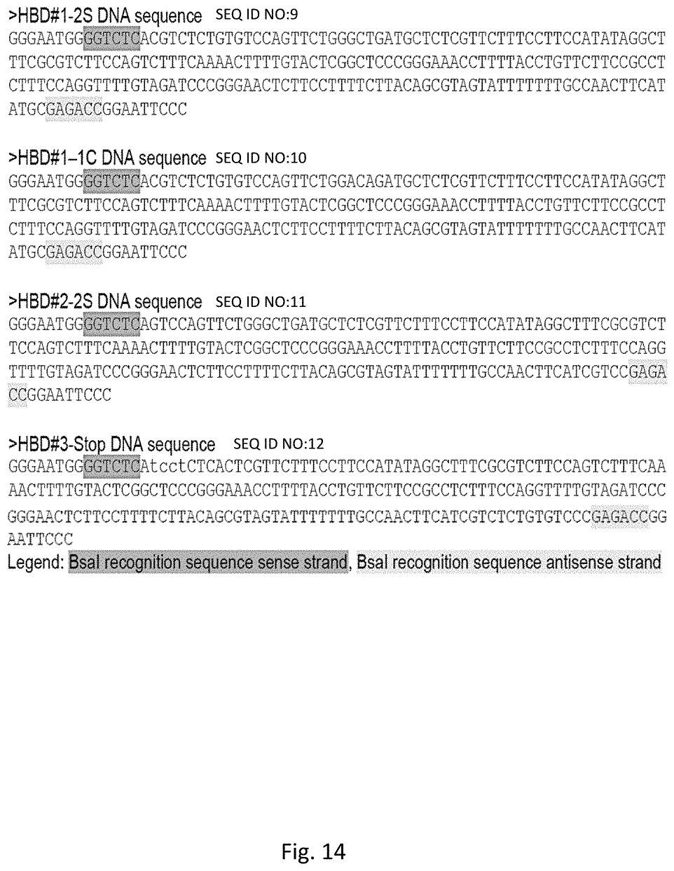

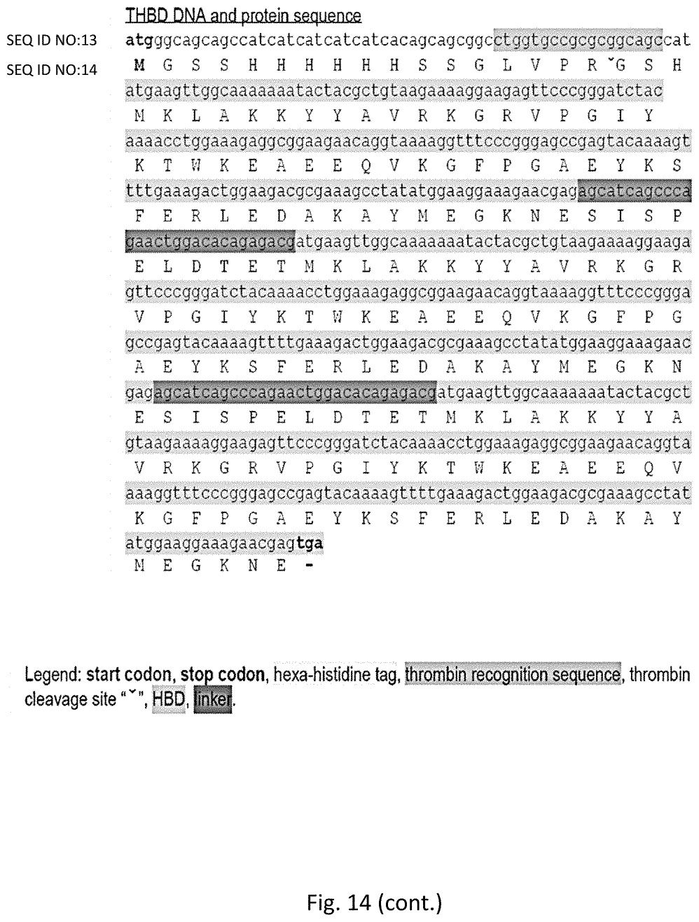

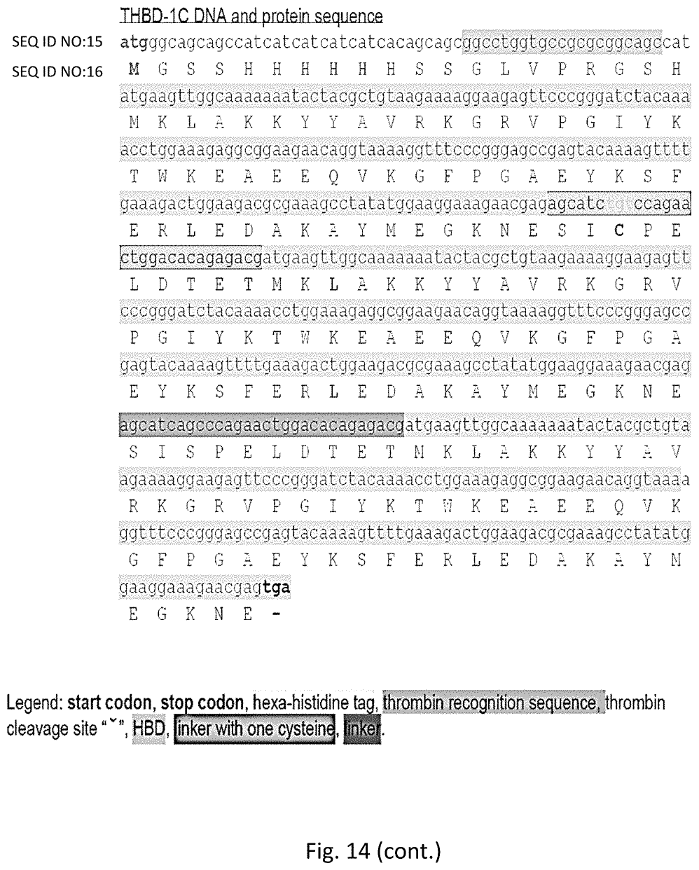

The THBD-1C synthetic gene differs from the THBD gene construct by the presence of a cysteine within the first linker. The rest of the sequence is identical to THBD, and therefore, the procedure to obtain the two genes are almost the same, apart from certain differences that are highlighted (FIG. 14). The cysteine in the THBD-1C is the only cysteine within the polypeptide sequence. This enables site-specific chemical modification (such as fluorescent dye attachment) using maleimide-sulfhydryl chemistry. The Golden Gate Assembly cloning strategy and BsaI enzyme were used to create the pET15b-THBD construct. Specifically, the THBD gene was inserted into a lab-constructed version of the pET15b vector that was domesticated for use with BsaI enzyme (FIG. 14). The THBD gene sequence contained three copies of the Tma HBD sequence. The gene, in order, encoded an HBD, a linker, the second HBD, the second linker, and the third HBD. THBD-1C differs from the THBD in the sequence of the first linker. The original linker sequence derives from Tma ribonuclease HI and contains two cysteines in position one and three. We created a modified linker in which the two cysteines were changed to serine, creating a Cysteine-free linker. The THBD-1C contains a single cysteine in the first linker region, obtained by changing the cysteine in linker position two to a serine. To implement the Golden Gate assembly cloning, the THBD and THBD-1C genes were assembled from three fragments that were purchased as synthetic dsDNAs, using gBlock technology (IDT Inc., Coralville, Iowa). Specifically, the THBD was provided in three different DNA constructs: HBD #1-2S, HBD #2-25, and HBD #3-Stop DNA sequences. Similarly, the THBD-1C was provided in three forms as HBD #1-1C, HBD #2-2S, and HBD #3-Stop. Each of the fragments were flanked by BsaI recognition sequences. BsaI digestion provided overhangs that enabled the ligated assembly of the three fragments into the pET15b vector, in the proper orientation and order. The Golden Gate Assembly protocol consists of a one-pot reaction, in which BsaI digestion (New England Biolabs, USA) and T4 ligation (New England Biolabs, USA) occur simultaneously. The product then was used to transform by electroporation ElectroMAX DH10B competent E. coli cells (Life Technologies) that allowed amplification of the pET15b-THBD and pET-15b-THBD-1C DNA constructs. The DNA sequences were verified by dideoxy sequencing (Genewiz).

THBD protein physical parameters: 205 aa, 23619 Da (after thrombin cleavage: 188 aa, 21737 Da)

THBD-1C protein physical parameters: 205 aa, 23634 Da (after thrombin cleavage: 188 aa, 21753 Da)

BsaI Domestication of pET15b Protein Expression Vector

The pET15b plasmid was modified to enable its use in the Golden Gate Assembly cloning strategy and BsaI enzyme. BsaI is a Type IIS restriction endonuclease that recognizes the 5′-GGTCTC-3′ sequence, and cleaves outside of the sequence (N1/N5), providing a four nucleotide 5′-overhang. The pET15b DNA sequence was modified in two places: (i) the single BsaI site located in the AmpR gene coding sequence was abolished (without inactivating the AmpR gene) and (ii) two BsaI sites were inserted into the pET15b multiple cloning site, allowing Golden Gate cloning using BsaI.

A single nucleotide mutation (d4781A→C) was accomplished using the Q5® Site-Directed Mutagenesis Kit (NEB, Beverley, Mass.) and the primers shown below, thus abolishing the BsaI site in the AmpR gene. The silent codon change (AGA to AGC) did not change the serine at that position. The mutation was verified by dideoxy sequencing (Genewiz). To introduce the BsaI cloning sites, an insertion of 24 nucleotides (ACCGATAATTTAGCTTTGGGGTCT) was engineered in the 329 to 330 region (329_\hat{ }330ins24), using the Q5® Site-Directed Mutagenesis Kit and primers listed below. The alteration was verified by dideoxy sequencing. The modified pET15b plasmid, named pET15b_BsaI was used for Golden Gate Assembly cloning of DHBD, THBD and THBD-1C.

### Primers for Mutagenesis

F_d4781A > C:
TGATACCGCGcGACCCACGCTC
R_d4781A > C:
TTGCAGCACTGGGGCCAG
F_329^{∧}330ins24:
gctttggggtctCATATGGCTGCCGCGCGG
R_329^{∧}330ins24:
taaattatcggtCTCGAGGATCCGGCTGCTAAC

### Example 6: Epitape Design, Features, and Prototype

Epiprinter detection of RNA employs custom DNA nanostructures, termed epitapes, that are designed to selectively capture the RNA target by complementary base-pairing. Detection of the bound RNA is accomplished by binding of the epiprinter to the DNA-RNA hybrid structure in the epitape, followed by covalent attachment of the epiprinter to the epitape. The modified epitape is then translocated across a solid-state nanopore, generating a signal that provides a permanent record of the original RNA binding event.

Achieving the optimally functionalized form of the epitape requires the development of a prototype that possesses key features with demonstrated functionality. The prototype is designed be readily assembled, taking advantage of the ability of DNA molecules with programmed sequence to self-assemble, and is tested and verified for its ability to engage in the RNA detection steps mentioned above. Establishing the identities and positions of the elements in the prototype epitape that provide optimal and consistent solid-state nanopore translocation behavior and signal generation is a key benchpoint for achieving the final, optimally functional structure. Finally, the epitape not only is used for RNA detection, but also DNA detection, protein detection, and detection of other molecules.

Described here are the features of the physical structure and sub-components of a self-assembling prototype epitape (proto-epitape). The essential functional features of the proto-epitape include the overall structure, the structure of the RNA-binding probe site, and the barcode element that identifies the epitape. The prototype epitape can be further evolved and elaborated for expanded functionality, including multiplexing, and the detection of DNA and protein.

First, the proto-epitape exhibits the physical structure as a stiff “string,” several hundred nanometers in length. The fundamental structure unit is a bundle of at least two parallel DNA double-helices, stabilized by periodic single-strand DNA crossover structures. There are several recent reports that presented evidence that the overall shape and the flexibility of linear DNA nanostructures can influence nanopore translocation behavior, and therefore also the quality of the output signal that records current change (Raveendran et al., 2018, ChemElectroChem, 5:3014; Wang et al., 2019, Nano Letters, 19:5661-5666). Given these findings, the epitape possessrd a sufficient stiffness to avoid, for example, the formation of knots, that can otherwise occur in single-stranded and double-stranded DNA molecules. DNA knots and related structures can generate artifactual signals during nanopore translocation (Plesa et al., 2016, Nature Nanotechnology, 11:1093-1097; Suma and Micheletti, 2017, Proceedings of the National Academy of Sciences, 114:E2991-E2997). To avoid these potential complications, the prototype epitape is a stiff linear structure, and is designed according to established techniques for creating DNA origami (Rothemund, 2006, Nature, 440: 297-302). In the DNA origami technique, a long, circular ssDNA (“scaffold”) is folded into the desired shape by its interaction with hundreds of short (30-40 nt) synthetic DNA oligonucleotides (“staples”). The most common scaffold DNA sequence is the circular, ssDNA genome of the bacterial virus, M13mp18 (˜7000 nt), of a defined sequence. It is important to note that other DNA sequences can be synthesized to function as scaffolds, and can be used according to the design and sequence needs. The staples interact with distal(non-contiguous) segments of the scaffold by Watson-Crick base-pairing. These interactions occur spontaneously in solution (since base-pairing is a spontaneous process) and the resultant nanostructure will be stabilized by a structurally specific network of double-helical domains and crossover junctions. Adjacent DNA helices in the structure will be held together by crossover junctions, consisting of staple strands exchanged between neighboring double helices. Specifically, a staple participating in a crossover initially interacts with one helix, then crosses to the neighboring helix, and binds to a complementary sequence in the second helix. The requisite number of helices (e.g. two or three), the density and periodicity of crossover junctions, and additional structural features such as terminal hairpin loops, are incorporated into the prototype epitape, using available computer-aided design and simulations. A properly designed proto-epitape exhibits an optimal, consistent signal-to-noise ratio during nanopore translocation, which in turn enables the sensitive and unambiguous detection of an attached epiprinter.

The proto-epitape contains functional elements that (i) enable detection of the target molecule, (ii) enable a barcode readout signal, thereby identifying the proto-epitape.

The RNA detection element (probe site) of the epitape consists of the extension of two single-strand sequences provided by two staples that are in close proximity. While the two staples help stabilize the proto-epitape structure, the two ssDNA extensions, both of which physically protrude from the proto-epitape structure, provide the probe site that binds the complementary target RNA sequence. The staples that provide the probe site sequences are synthesized to recognize a unique sequence of the target RNA (e.g. viral RNA).

The initial structure chosen for the probe site structure reflect the design described by (Ke et al., 2008, Science, 319:180-183), wherein two juxtaposed, 10-nucleotide single-strand sequences protrude from the nanostructure, and thus can form a DNA-RNA hybrid upon binding the RNA target. Binding of the added epiprinter to the proto-epitape, followed by covalent attachment of the epiprinter to the epitape results in a physical enlargement of the epitape at the probe site, that can be detected by a transient current drop during nanopore translocation. For a given RNA target, the probe length is determined that provides optimal (i) sequence specificity and binding affinity for the RNA target; (ii) epitape binding affinity and covalent reaction; and (iii) nanopore translocation signal. Alternative probe site structures also can be used. For example, the extremities (termini) of the epitape may be designed to bind the target RNA, such that epitapes with bound RNA could be joined end-to-end manner. The extended structure will in turn generate a unique nanopore signal that reflects the prior binding of RNA.

The barcode element is comprised of specific combinations of multiple individual signaling units. An individual signaling unit will be a localized DNA structure in the proto-epitape that will cause a localized steric enlargement of the epitape, detectable by nanopore translocation in a unique, consistent manner. The DNA secondary structures can protrude from the epitape, or be provided by other molecular entities that are covalently attached to the epitape, such as a small protein, or a molecular polymer (e.g. polyethylene glycol). DNA secondary structures could be a DNA hairpin (Chen et al., 2019, Nano Letters, 19:1210-1215), DNA dumbbell structures (Bell and Keyser, 2016, Nature Nanotechnology, 11:645-651), or designed enlargements of short stretches of the epitape itself. For example, to create a hairpin unit, a specific staple in the epitape can be lengthened to include a sequence that forms a hairpin. The resulting hairpin protrudes from the epitape, and is anticipated to create a transient downward spike in the current signal during translocation. A signaling unit also may be created by covalently attaching a molecular entity to the epitape. To accomplish this, synthetic DNA staples are commercially modified at specific nucleotides with a broad range of chemical groups, including those that allow click-chemistry, or sulfhydryl-maleimide reactions. Thus, a polyethylene glycol (PEG) molecule, modified with the complementary click-chemistry group, allows its attachment to specific points in the proto-epitape.

The barcode contains several of these signaling units in a specific array or order on the proto-epitape. The optimal structure of a signaling unit is defined as the one that allows the barcode to provide an unambiguous, easily interpretable output signal. Important parameters to evaluate are the number of signaling units that define a specific signal, the spacing between the signaling elements that allows barcode differentiation, and incorporation with the proto-epitape. Successful barcode design enables implementation of multiplexing in RNA detection and also allows determination of the directionality of the epitape during translocation.

### Example 7: Using Epitape Technology to Detect DNA

Adapting epiprinting to detect DNA (for example, viral DNA), broadens the range of biomarkers that can be identified (e.g. disease vectors such as mosquitoes, and species/organism identification). The modified epitape also can identify DNA barcodes that are used in a variety of applications. A straightforward modification to the epiprinting technology enables DNA sequence detection. Here, the epitape is modified to have the probe site DNA “arms” replaced by RNA arms. This is accomplished by replacing the two DNA staples that provide the probe site arms with two RNA oligonucleotides with single-stranded probe arms that maintain the original sequences. The RNA oligonucleotides with the required sequence and length are chemically synthesized by a commercial provider (e.g. IDT Technologies). The RNA oligonucleotides are further modified to contain alkynyl groups on the 5-carbons of selected uridine residues in the RNA “arms”, enabling click chemistry with the epiprinter. The remainder of the epitape structure otherwise remains the same.

To determine whether a sample contains a DNA sequence of interest, the sample is combined with the epitape having RNA arms with sequences complementary to the target DNA sequence. If the target DNA is present, and complementary base-pairing occurs, an RNA-DNA hybrid is formed at the epitape RNA probe site. Addition of the epiprinter, followed by click chemistry-enabled covalent bond formation, creates a covalent link between the epitape RNA probe sequences and the epiprinter. The sample is then subjected to nanopore analysis. The attached epiprinter provides a characteristic output signal during epitape translocation, in the same manner described for the RNA detection methodology, creating a permanent recording of the DNA binding event. Thus, for the proposed DNA detection technology, the fundamental event of formation of a DNA-RNA hybrid, followed by epiprinter addition and covalent bond formation, remains unchanged.

Epitape technology for DNA detection is used for detection of disease biomarkers (DNA of viruses, microbes, other pathogens), detection of DNA barcodes—identification of DNA barcoded items, and determination of species of organisms of interest (e.g. disease vectors) otherwise challenging to identify.

### Example 8: Using Epitape Technology to Detect Proteins and Other Molecules

Adapting epitape technology to detect protein significantly expands the range of detectable biomarkers beyond RNA and DNA. Proteins are biomarkers for many disease conditions, including cancer, and additional applications are envisioned for epiprinting detection of protein in the food and agricultural industry, as well as in basic and applied research. Protein detection by epiprinting exploits the exquisite specificity and high affinity of antibodies for proteins.

The epitape is modified to carry an RNA-containing probe site (the same as that for the proposed DNA detection technology). The RNA probe site allows sequence-specific DNA binding, and is also modified to engage in click chemistry. The epiprinter is the same as used for RNA detection. The antibody is modified to carry a covalently-attached DNA oligonucleotide of sequence that is complementary to the epitape RNA probe site sequence. This antibody-DNA conjugate provides the physical link that connects the antibody detection of protein to sequence-specific DNA-RNA hybrid formation. The antibody-oligonucleotide covalent conjugate is prepared by established methods (Rosen et al. 2014, Nature Chemistry, 6:804-809; Trads et al., 2017, Accounts of Chemical Research, 50:1367-1374), using purified antibody and synthetic DNA oligonucleotide, and coupling chemistry.

The sample containing the protein of interest is incubated first with an unmodified primary antibody that recognizes the protein, allowing forming a stable primary antibody-antigen complex. Excess antibody is removed, and the antibody-DNA conjugate is added that recognizes the primary antibody and forms a stable complex. Removal of excess unbound antibody-DNA is followed by addition of the epitape, which binds the DNA attached to the secondary antibody, creating a DNA-RNA hybrid at the RNA probe site. The epiprinter is added, which binds the DNA-RNA hybrid structure, and allowing click chemistry to create a covalent bond between the epiprinter and the RNA strand of the epitape. This reaction provides a permanent recording of the primary antibody binding to the protein. The epiprinter-modified epitape is then subjected to nanopore analysis.

Epitape technology can be further extended to detect other molecules and biomolecules of interest, by incorporating the use of aptamers in place of antibody-DNA conjugates. Aptamers are nucleic acid (DNA or RNA) structures, developed using in vitro selection technologies to recognize and bind a wide range of molecules (Du et al., 2013, Accounts of Chemical Research, 46:203-213; Mehlhorn et al., 2018, Biosensors, 8; Tan et al., 2020, Advances and Perspectives. Angew. Chem. Int. Ed., Accepted Author Manuscript. doi:10.1002/anie.202003563). Pre-existing aptamers can be used, or created de novo for the target molecule of interest, then easily adapted to epiprinter use. Here, the aptamer contains an additional DNA sequence that is complementary to the epitape with an RNA probe site. Addition of the modified aptamer to the sample under investigation enables tight binding to the molecule of interest, if present. Addition of the epitape allows binding to the complementary DNA sequence in the aptamer, forming an DNA-RNA hybrid. Addition of the epiprinter, followed by click chemistry, provides covalent attachment of the epiprinter to the epitape, providing a permanent recording of the aptamer binding to the molecule of interest. Nanopore analysis provides the recordable output.

Epitape technology for detection of proteins and other molecules is used for detection of protein biomarkers for diagnosis, prognosis and therapy response evaluation of cancer, detection of proteins for testing of pathogen infections (viral, bacterial) and health and environmental surveillance, detection of proteins in research laboratory settings, and detection of proteins in industrial quality control checks (food and biotechnology).

### Example 9: Multivalent Forms of the Ribonuclease H1 Hybrid Binding Domain (HBD) as High Affinity Binders of RNA-DNA Hybrids

The experiments described herein demonstrate that joining two HBDs by a 10aa linker creates a protein with a ˜280-fold higher affinity for hybrids compared to the single HBD (KD ˜29 nM and ˜8 μM, respectively). The substantial increase in affinity indicates a cooperation of the linked HBDs, since if the binding events were independent there would be only a two-fold increase in affinity (˜4 μM) (Shamoo et al., 1995, Nucleic Acids Res. 23, 725-728). The KD for the DHBD-hybrid complex is far from the theoretical maximum affinity (predicted KD ˜70 pM), as would apply if the binding free energies of the linked HBDs were fully additive. Specifically, the predicted KD equals (1/KA)², where KA is the equilibrium association constant for the SHBD-hybrid complex. The ˜400-fold difference between the experimental KD and the theoretical maximum KD reflects the influence of the linker in preventing full cooperativity. After one HBD is bound the linker confines the second HBD to provide an “active” local concentration of 1.44 mM, which is independent from the solution concentration (Krishnamurthy et al., 2007, J. Am. Chem. Soc. 129, 1312-1320). This is ˜700-fold lower than the 1 M concentration that would apply if the HBD binding free energies were additive (Shamoo et al., 1995, Nucleic Acids Res. 23, 725-728). The active local concentration of the tethered but unbound HBD reflects the volume available to the domain, which would correspond to a sphere of ˜65 Å radius. Here, the radius is the distance between the centers of mass of the two HBDs, where the HBD diameter is estimated to be 30 Å (Nowotny et al., 2008, EMBO J. 27, 1172-1181) and the linker is assumed to exhibit extended random coil behavior, with a ˜3.5 Å inter-residue distance.

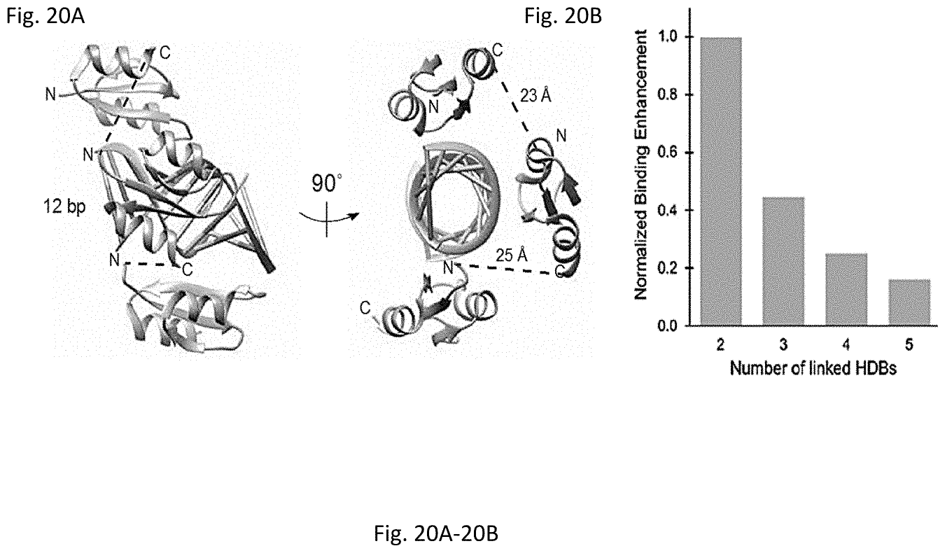

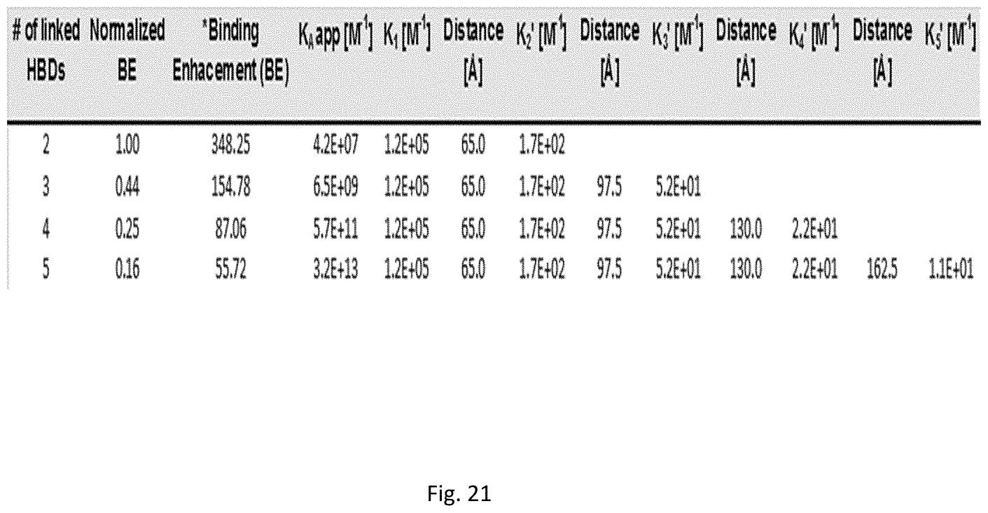

The theoretical hybrid affinities of the multi-HBD proteins was estimated by considering the interdomain distance, which depends upon the linker length (Shamoo et al., 1995, Nucleic Acids Res. 23, 725-728; Crothers et al., 1972, Immunochemistry, 9, 341-357). A theoretical KD of ˜24 nM was obtained for the DHBD-hybrid complex, which is comparable to the experimental KD of ˜29 nM, and indicates the linker amino acids conformations and length support the expected level of cooperative binding. A computational prediction obtained using PEP-FOLD (Shen et al., 2014, J. Chem. Theory Comput. 10, 4745-4758) indicates that the majority of the linker amino acids can adopt a random coil or extended conformations. FIG. 20A highlights the distances (average ˜24 Å) between the C-terminus and N-terminus of adjacent HBDs bound to a hybrid (Nowotny et al., 2008, EMBO J. 27, 1172-1181). As this distance is less than the ˜35 Å length for a fully-extended 10aa linker, the DHBD linker therefore would not be expected to perturb the binding of the individual HBDs, in accord with the experiments. A shorter linker in principle could further enhance binding affinity by increasing the local active concentration of the unbound domain, but shorter lengths may impose steric constraints or otherwise cause conflicting domain-domain interactions that would perturb binding of each HBD. Liang and coworkers recently reported the hybrid-binding affinity of a recombinant protein consisting of two HBDs (from human RNase H1) connected by a pentaglycine linker 21. A KD of 16.5 nM was determined for a hybrid:HBD complex, and a KD of 10.4 nM for the hybrid:double-HBD complex. It is possible that the minor increase in binding enhancement observed for the double-HBD construct reflects a predicted length of ˜17.5 Å for the 5aa linker, which may have introduced HBD-HBD-domain interactions that interfered with optimal engagement of the hybrid.

The SPR analyses described herein show that the linking of the third HBD confers an ˜80-fold enhancement of affinity relative to the DHBD. This is a smaller enhancement (˜25% increase) than the enhancement provided by joining two HBDs. To gain a further understanding, a theoretical model was used to determine the relative enhancement of hybrid affinity of proteins consisting of up to five linked HBDs. The plot in FIG. 20B shows the additional binding enhancement is expected to increase by less than 50% when three or more domains are linked. The decrease in enhancement may be associated with the increased inter-domain distance in multiple linked domains. The additional linkers and domains would contribute to the interdomain distance, further reducing the local active concentration of the unbound domain. A reduction in local concentration is reiterated with the incorporation of additional domains, with each additional domain providing an increase in affinity, albeit relatively reduced.

Analysis of the binding kinetics reveals that linking HBDs enhances affinity primarily by slowing the dissociation rate constant (˜14-fold decrease), and to a lesser extent by increasing the association rate constant (˜4-fold increase). It is relevant to note that Park and coworkers reported that removing either of two RNA binding domains in HuD, a multi-domain RNA-binding protein, slows the dissociation rate by ˜50-fold, and increases the association kinetics by ˜7-fold (Park et al., 2004, Mol. Cell. Biol. 24, 6888 LP-6888).

The SPR and EMSA analyses provided complementary information on the stoichiometries of the hybrid complexes involving SHBD, DHBD, and THBD. While the micromolar KD value and rapid dissociation kinetics of the SHBD-hybrid complex did not permit observation of discrete higher-order complexes in EMSA, the SPR analysis indicates that a 21 bp hybrid can accommodate up to six SHBDs. This stoichiometry is consistent with a crystallographic study of a human HBD-hybrid cocrystal (Nowotny et al., 2008, EMBO J. 27, 1172-1181), which showed that a quasi-continuous 24 bp helix formed by the coaxial stacking of two 12 bp hybrids can accommodate six HBDs. Even though an HBD physically spans ˜9 bp (see FIG. 20A), the double-helical nature of the hybrid permits binding of up to six HBDs by permitting the rotational translocation and thus separation of adjacent HBDs. The EMSA and SPR both show that the 21 bp hybrid can bind two DHBDs, with the gel shift assay indicating that the DHBD concentration should reach at least ˜10 μM to observe the second complex. While it is formally possible that a third DHBD could be accommodated by a 21 bp hybrid, such a complex was not detected, which may reflect its relative instability. EMSA analysis reveals that up to two THBDs can bind the 21 bp hybrid. Here, a 2:1 THBD:hybrid complex would be equivalent to a 6:1 SHBD:hybrid complex. Thus, the EMSA data indicate that the two linkers in the THBD can allow close-packed binding of individual HBDs in the same manner as seen in the crystal structure. In contrast, SPR detected the binding of only one THBD. It is not clear why the SPR did not detect a 2:1 complex, but the apical loop structure of the hairpin hybrid could interfere with the binding of a second THBD.

In conclusion, high-affinity binders for RNA-DNA hybrids can be achieved by linking multiple copies of an HBD derived from a bacterial RNase H1. The KD for the protein consisting of three linked HBDs is in the sub-nanomolar range, which is comparable to the hybrid affinity of the single-chain variable fragment (scFv) of the S9.6 antibody (Phillips et al., 2013, J. Mol. Recognit. 26, 376-381), and the affinity of a neomycin-methidium conjugate (Shaw et al., 2008, Bioorg. Med. Chem. Lett. 2008, 18, 4142-4145). Further binding affinity is expected to be achieved by linking additional domains, but with a predicted diminished enhancement of affinity. The HBD based protein constructs and modified versions thereof are expected to be useful in a variety of applications in nucleic acid detection technologies.

The materials and methods used in the experiments are now described.

Preparation of Synthetic Gene Constructs

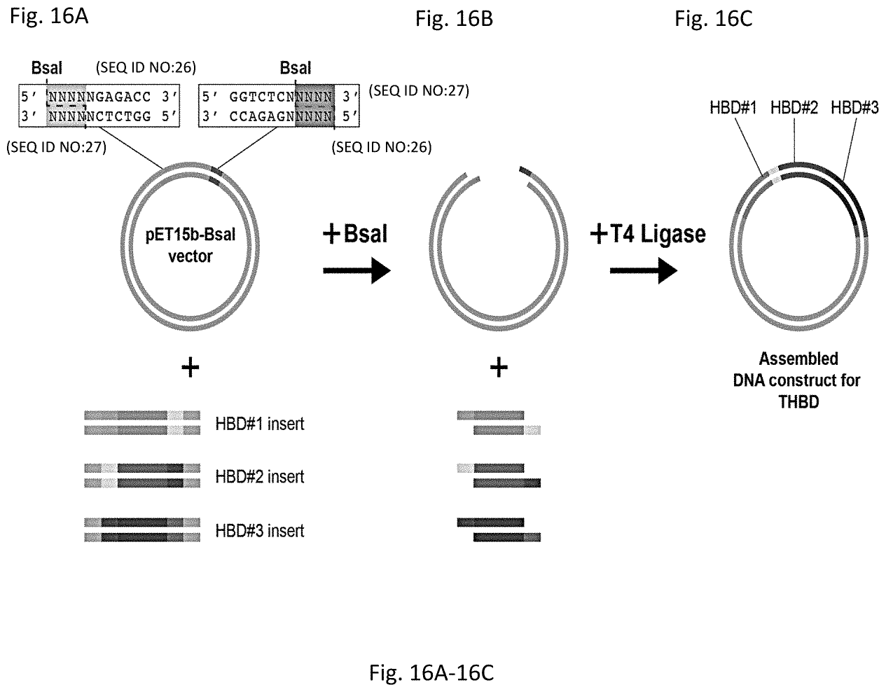

The HBD sequence (SHBD) of the RNase H1 gene from Thermotoga maritima was amplified from genomic DNA (ATCC) using Vent DNA polymerase and a specifically designed primer pair. The purified DNA was cleaved with NdeI and BamHI and ligated to NdeI, BamHI-cleaved pET-15b plasmid (MilliporeSigma). The DHBD and THBD genes were assembled from synthetic double-stranded DNAs and inserted into a modified pET-15b plasmid (see FIG. 16), using the Golden Gate Assembly approach (Engler et al., 2008, PLoS One, 3, e3647) as based on BsaI Type IIs restriction enzyme, and T4 DNA ligase (see FIG. 16 for gene assembly details). The assembly reaction was used to transform competent E. coli 10-beta cells (NEB) according to the supplier's instructions, under growth selection of LB agar plates containing 100 μg/ml ampicillin. Recombinant plasmids were purified using the Plasmid Midi Plus Purification kit (Qiagen), and DNA sequences were verified by sequencing (Genewiz).

Recombinant Protein Production and Purification

E. coli BL21(DE3) cells (NEB) freshly transformed with the specific recombinant plasmid were inoculated into LB broth (100 mL) containing ampicillin (100 μg/mL), and incubated at 3TC with shaking (250 rpm). Protein overexpression was induced at 0.4-0.5 OD₆₀₀ by 1 mM IPTG, followed by further incubation with shaking for 4 hr at 3TC. Cells were harvested by centrifugation (6000×g, 15 min, 4° C.), and the pellet was stored at −80° C. until further use. The pellet was resuspended in Buffer A (50 mM NaH₂PO₄, 500 mM NaCl, 3 mM TCEP, 60 mM Imidazole, pH 7.4), and subjected to on-ice sonication (Misonix Microson XL cell disruptor), applying 15 cycles of a 30 sec sonication, with 30 sec between bursts. The lysate was centrifuged (18000×g, 20 min, 4° C.), then applied to a 1 mL HisTrap™ Fast Flow Crude column (Cytiva) mounted on an AKTA Explorer FPLC (Cytiva), and equilibrated using Buffer A. After washing with Buffer A the column was equilibrated in Buffer B (50 mM NaH₂PO₄, 150 mM NaCl, pH 7.4) and the column-immobilized protein incubated overnight at room temperature with 9 units of human thrombin (Novagen). The thrombin-treated protein was eluted with Buffer B and subjected to size-exclusion chromatography (SEC) in Buffer B using a Superdex 75 Increase 10/300 gl column (Cytiva), and applying a 0.4 mL/min flow rate. Protein concentrations were determined by measuring absorbance at 280 nm with a Nanodrop 1000 (Thermo) and using extinction coefficients calculated by the ProtParam tool (expasy.org/protparam), based on the protein sequence. Protein purity was assessed by electrophoresis of 5 μg samples using a 12% SDS-PAGE NuPAGE gel (Thermo), followed by Bio-Safe Coomassie staining (BioRad). Quantification of gel band intensity was performed with Image J (imagej.nih.gov).

Electrophoretic Mobility Shift Assay

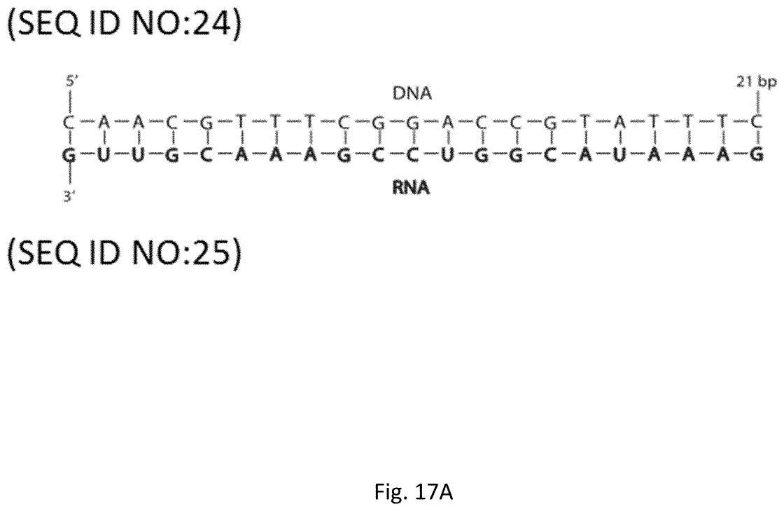

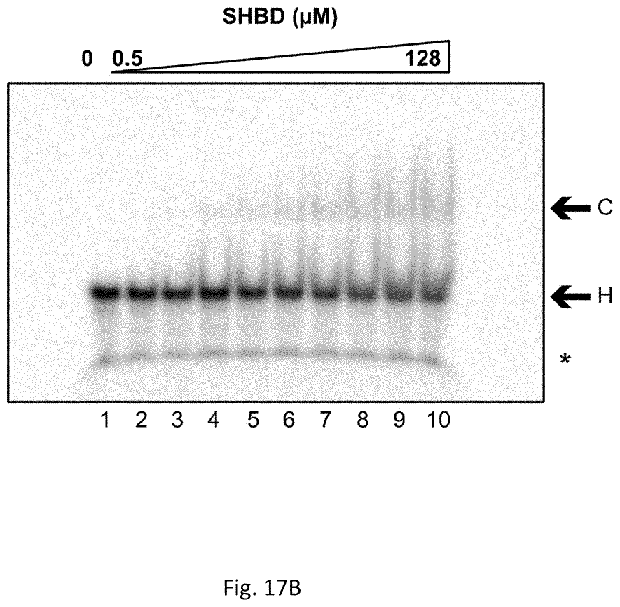

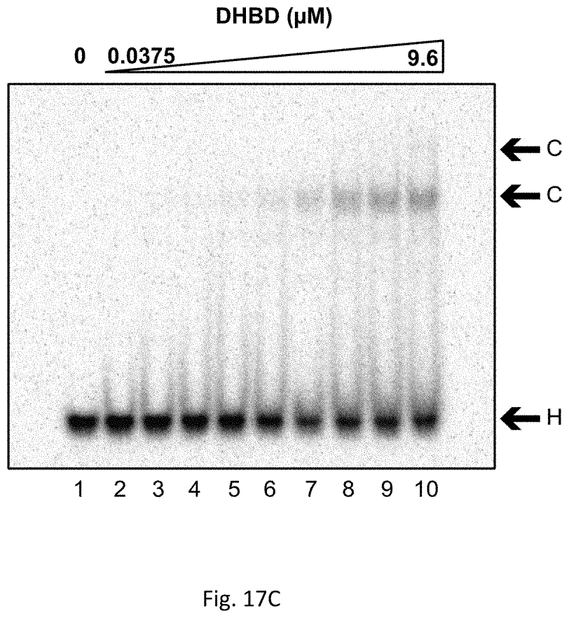

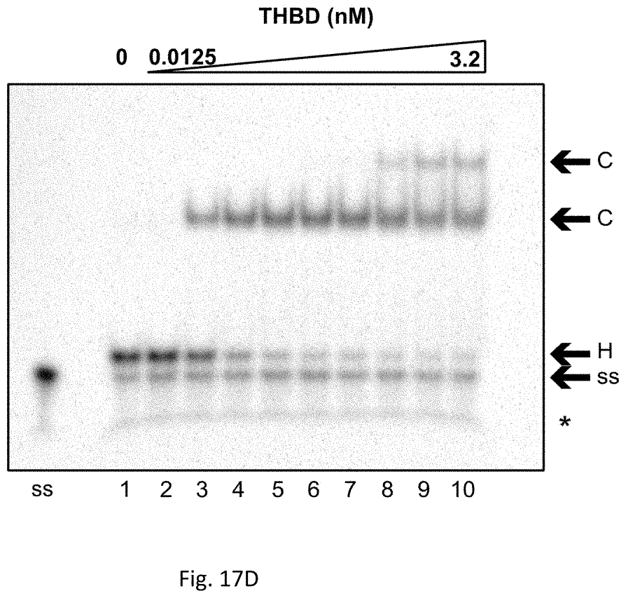

The RNA oligonucleotide was labeled at the 5′-end using [g-³²P] ATP (3000 Ci/mmol) (PerkinElmer) and T4 polynucleotide kinase according to the supplied instructions. The labeled RNA was purified using an Oligo Clean & Concentrator Kit (Zymo Research), following the supplier's protocol. Labeled RNA purity was verified by gel electrophoresis, and radioactivity was quantified by scintillation counting. Hybrid (FIG. 17A) formation was carried out in 60 mM Tris, 100 mM NaCl, 0.5 mM EDTA (pH 8) by mixing one part of ³²P labeled RNA and five parts of complementary DNA. The annealing reaction was heated for 1 min at 90° C. and then held at room temperature for 30 min. Binding reactions were prepared by combining increasing concentrations of protein with a fixed hybrid concentration in a 60 μL reaction volume, and using a buffer of 50 mM NaH₂PO₄, 150 mM NaCl, 0.5 mM TCEP, 5% Glycerol (pH 7.4). The reactions were incubated for 30 min at room temperature, then loaded onto a 8% polyacrylamide gel (80:1, Acrylamide:Bisacrylamide in 0.5×TBE buffer) and electrophoresed at 150 V for 2-2.5 hours. The ³²P signal was visualized by phosphorimaging using a Typhoon 8000 scanner (Cytiva).

SPR Analysis

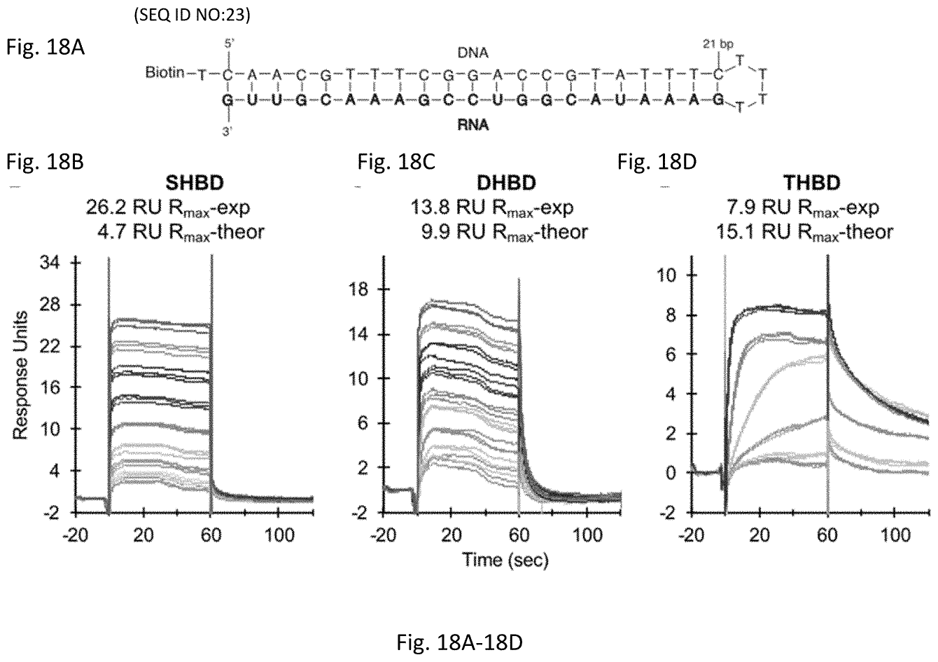

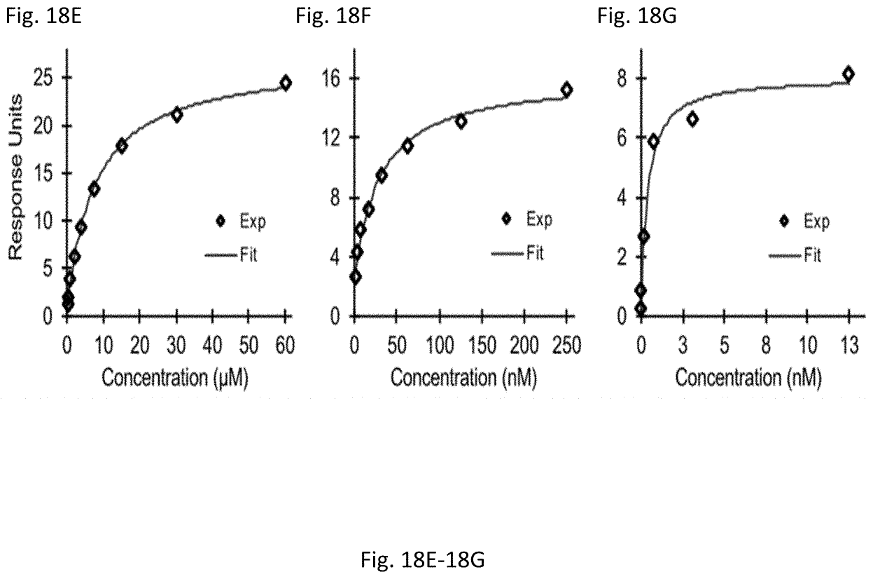

SPR analysis of protein binding to a model hybrid used a Biacore™ T200 instrument (Cytiva). A CM4 sensor chip (Cytiva) was modified with neutravidin (Pierce) using the amine coupling kit (Cytiva). The neutravidin was diluted to 0.1 mg/mL in 10 mM sodium acetate (pH 5), then reiteratively injected (20 μL/min) until the neutravidin immobilization levels attained ˜1000 RU. The neutravidin-modified CM4 sensor chip then was functionalized with an RNA-DNA hybrid hairpin carrying a biotin moiety at the 5′ end (FIG. 18A). The hybrid was diluted to 0.1 or 1 μM in 50 mM NaCl, 10 mM Tris (pH 8). Each solution was reiteratively injected (2 μL/min) until an immobilization level of ˜10 RU was achieved. The concentration of the immobilized hybrid was estimated to be 694 nM by using the conversion, 1 RU=1 pg/mm² (Myszka et al., 1998, Biophys. J. 75, 583-594) and applying a matrix height of 0.0001 mm. The hybrid-to-hybrid distance was estimated to be 133 nm by using the equation, d=1.18/C^{1/3}, where d is in nanometers and C is the molar concentration (Erickson et al., 2009, Biol. Proced. Online, 11, 32-51).

Serial dilutions of purified protein were prepared in analysis buffer (100 mM NaCl, 50 mM NaH₂PO₄, 1 mM EDTA, 0.05% (v/v) Tween-20, pH 7.4) and were injected at a flow rate of 50 μL/min. Experiments were performed in triplicate at 25° C. Data transformation of the primary sensorgrams and determination of the kinetic and equilibrium affinity parameters were accomplished using the global data analysis tool available in BiaEvaluation 3.2 software (Cytiva) and applying a 1:1 binding isotherm model.

Homology Modeling of the Thermotoga maritima HBD Structure.

Information on the structure of the Thermotoga maritima RNase H1 HBD (Tm-HBD) can provide useful insight on aspects of the HBD-hybrid interaction. A comparative homology modeling approach was used since a crystallographic structure is lacking. The human RNase H1 HBD (Hs-HBD) was used as a template, as determined by x-ray diffraction in a complex with a 12 bp DNA-RNA hybrid (PDB ID: 3BSU) (Nowotny et al., 2008, EMBO J. 27, 1172-1181). A blastp alignment revealed that the sequences of Tm-HBD (accession no. AHD18801) and Hs-HBD (accession no. EAX01061) can be aligned, without gaps, over a 44 amino acid segment, with a 43% sequence identity and a similarity score of 63%. Structural homology modeling was performed using the Swiss-Model web server (Waterhouse et al. 2018). The modeled Tm-HBD structure fully adopted a three-stranded β sheet and two α helices, arranged in a bbaba topology, as with the Hs-HBD.

Predicted Affinity of Multidomain Proteins

The theoretical model described by Crothers and Metzger² that later was adapted for divalent binders by Shamoo and coworkers³ was used for predicting the affinity of multidomain proteins. According to the model the affinity is calculated for the subsequent binding of the second HBD or in general the next unbound HBD of a multi-HBD protein to a nucleic acid ligand that is already bound by the first HBD or in general firstly bound HBDs of the same protein. The affinities were calculated based on equation (I) and by applying the SHBD experimental K_{A} 1.2 10⁵ M⁻¹ that reflects affinity of the isolated HBD has for that nucleic acid ligand. The effective affinity (K′) of the tethered and unbound domain is:

Kᵢ′=3V(Kᵢ)/4πr³N (I)

Kᵢ is the affinity that would be observed for the tethered binding domain had it bound first, “r” is the mean free radius linking the two binding domains, and N is the number of particles per volume (V) in the standard state (i.e., 1 M) defined for the binding of the first binding domain (K₁). “r” corresponds to the distance between the centers of mass of the two neighboring binding domains. The distance calculation assumes the linker is an extended random coil with a residue distance of 3.5 Å and that each HBD has a radius of ˜15 Å⁴. The overall apparent affinity (KAapp) of the multi-HBD protein was calculated by equation:

$${K}_{A} app = {Ω}_{n} {\prod }_{i = 1}^{n} {K}_{i}^{′} \qquad ( II )$$

where n is the number of domains compositing the protein, and Ωₙ, equal to n!, is for degeneracy arising from interchange of the HBDs binding order to the nucleic acid lattice. Although multiple binding schemes can occur during the binding event, a complete description of the binding dynamics is not the purpose of this analysis. The case where the HBDs binding consecutively was the only case considered, to provide a rule of thumb for the effect of the number of domains on overall affinity or avidity of the multi-HBD protein.

Results

Design and Production of Multi-HBD Proteins

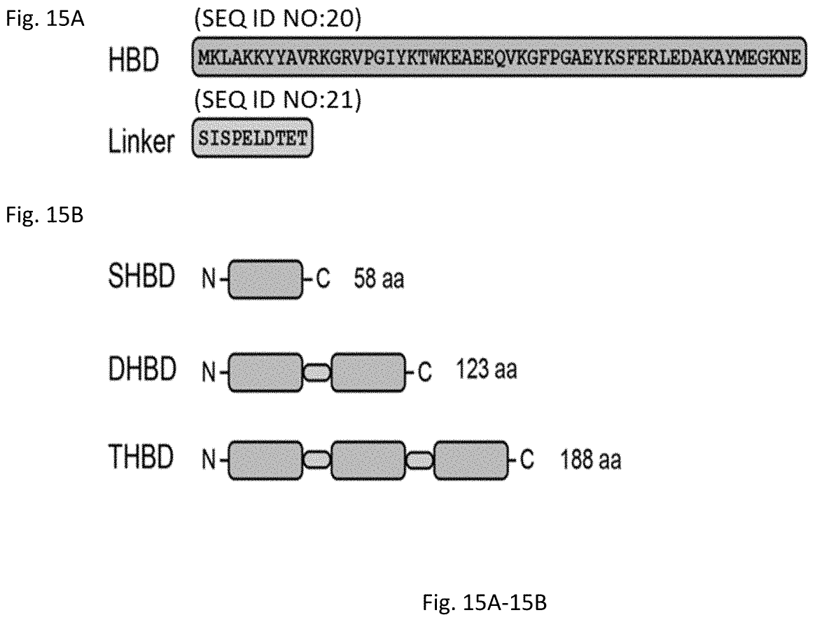

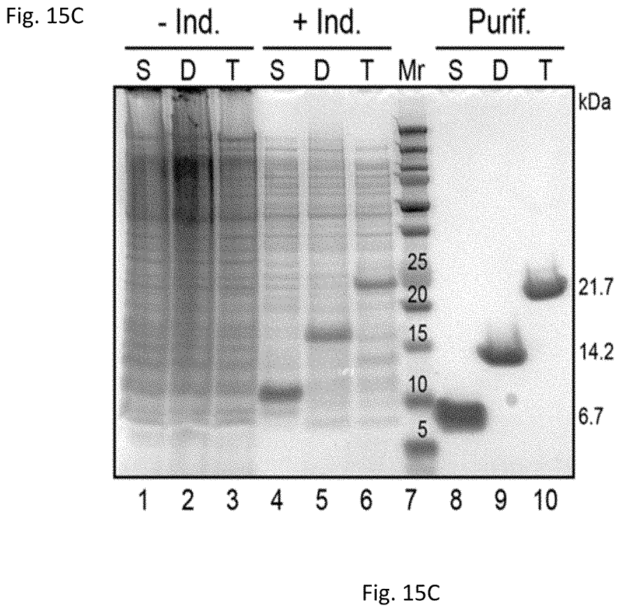

The HBD selected to develop multi-HBD proteins was that of Thermotoga maritima (Tm) RNase H1, which carries a single copy of the domain that is connected by a short linker to the C-terminal nuclease domain. The hybrid binding ability of the Tm-HBD in isolated form was reported by Kanaya and coworkers (Jongruja et al., 2010, FEBS J. 277, 4474-4489). While there is no direct structural information on the Tm-HBD, pairwise sequence alignment reveals that it shares 43% and 48% sequence identity with the human RNase H1 HBD and the yeast RNase H1 N-terminal proximal HBD, respectively. The availability of direct structural information on the human and yeast HBDs (Nowotny et al., 2008, EMBO J. 27, 1172-1181; Evans et al., 1999, J. Mol. Biol. 291, 661-669) enabled homology modeling of the Tm-HBD structure. Analysis of the structure revealed that conserved residues involved in direct contacts with the nucleic acid also were conserved in the corresponding positions in Tm-HBD. The Tm-HBD sequence chosen for cloning included residues M1 to E55, and the 10aa linker selected for joining HBD modules was that of Tm-RNase H1 (FIG. 15A). Since the linker supports HBD function in Tm-RNase H1 it was also expected to support hybrid binding by the multi-HBD proteins. The cysteines at linker positions 1 and 3 were replaced by serine in order to avoid protein crosslinking and sulfhydryl oxidation.

DNA sequences encoding a single HBD (“SHBD”), or two HBDs and a linker (“DHBD”), or three HBDs with two linkers (“THBD”) were synthesized, then assembled (see FIG. 15A and FIG. 16) using the Golden Gate assembly strategy (Engler et al., 2008, PLoS One, 3, e3647). This assembly method was used, as construction of genes with repetitive sequences using PCR-based approaches posed practical challenges (Hommelsheim et al., 2015, Sci. Rep. 4, 5052). The synthetic genes encoded proteins that contain an N-terminal hexa-histidine affinity tag and a thrombin cleavage site, both of which derived from the pET-15b vector. Growth of E. coli transformants followed by IPTG addition resulted in the appearance of proteins of sizes consistent with their predicted molecular weights (FIG. 15C, lanes 4-6). Protein production did not affect cell growth, and the proteins were present in the soluble portion of sonicated cell extracts. The proteins were purified by Ni(II) affinity chromatography, which included an in-column thrombin treatment followed by size exclusion chromatography of the eluted protein. The yield of the purified SHBD, DHBD, and THBD proteins from 100 mL culture was 13 mg/L, 10 mg/L, and 2 mg/ml, respectively, with ˜70% apparent purity as estimated by SDS-PAGE (FIG. 15B and FIG. 15C, lanes 8-10). The predicted pI values for the SHBD, DHBD, and THBD are 9.4, 9.2, and 9.1 respectively.

Electrophoretic Mobility Shift Analysis of Hybrid Binding by Multi-HBD Proteins

Electrophoretic mobility shift assays (EMSA) were performed to assess the hybrid binding behaviors of the purified recombinant multi-HBD proteins. The assays employed a 21 bp duplex hybrid (FIG. 17A), in radiolabeled form. Based on crystallographic data (Nowotny et al., 2008, EMBO J. 27, 1172-1181), the hybrid was expected to accommodate binding of up to six copies of the SHBD, three copies of the DHBD, and two copies of the THBD, ignoring any effect of the linker. The SHBD titration experiment reveals a discrete complex (FIG. 17B), the amount of which increases with protein concentration, and presumably corresponds to the binding of a single SHBD. Higher SHBD concentrations led to the appearance of distributed ³²P-radioactivity of slower mobility that most likely reflects the formation of less stable complexes containing multiple HBDs. The non-equilibrium nature of the gel shift assay may explain the inability to observe higher order complexes in discrete form.

The DHBD titration experiment (FIG. 17C) reveals two complexes, with the slower-moving complex visible at the higher protein concentrations. Based on the faster-moving complex, and assuming a 1:1 complex stoichiometry, the DHBD exhibits an increased hybrid affinity by over an order of magnitude compared to the SHBD. The THBD titration experiment (FIG. 17D) also reveals two complexes, with the slower complex visible at the higher concentrations. These data indicate that incorporation of a third HBD further enhances hybrid affinity by at least an order of magnitude, and that the 21 bp hybrid can bind up to two THBDs. FIG. 17D also shows that, as expected, the THBD does not bind the 21 nt ssRNA, which migrates in the gel slightly faster than the hybrid.

Surface Plasmon Resonance Analysis of Hybrid Binding by Multi-HBD Proteins

Surface Plasmon Resonance (SPR) was performed to gain quantitative information on the hybrid binding affinities and kinetics of the multi-HBD proteins. A 21 bp hybrid with the same sequence as the EMSA substrate was synthesized in the form of a hairpin stem-loop (FIG. 18A), which allowed formation of a homogenous probe population on the chip surface. The hybrid was immobilized on a neutravidin-modified Biacore CM4 chip surface via a 5′-biotin linker (see Materials and Methods). The CM4 chip and neutravidin were chosen so as to minimize any nonspecific, charge-related interactions. Low hybrid immobilization levels (˜10 RU) were established, to minimize mass transfer effects (Myszka et al., 1998, Biophys. J. 75, 583-594). The estimated inter-hybrid distance of ˜133 nM would prevent protein cross-binding, as the largest protein (THBD) has a 16 nm maximal linear dimension. Purified proteins were injected onto the hybrid-containing chip surface, with subsequent buffer injections serving to initiate complex breakdown. The change of mass on the chip surface due to protein binding was recorded as response units (RU), and protein association and dissociation events were recorded as sensorgrams that were analyzed. The retention of functional integrity of the hybrid hairpin was indicated by the consistency of Rₘₐₓ−exp values across triplicate experiments.

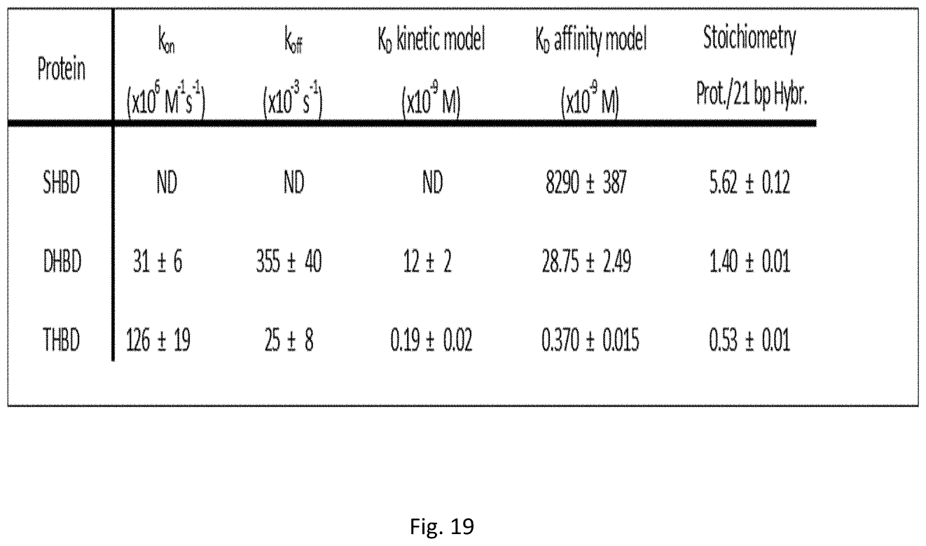

FIGS. 18B-18D displays sensorgrams for protein titration experiments involving the SHBD, DHBD, and THBD. In order to allow accurate K_{D} determination, binding responses were obtained using protein concentrations spanning the 0.1-10×K_{D} range. The responses for the DHBD and the THBD also were measured at higher protein concentrations (>100×K_{D}), where the binding responses continued to increase without apparent saturation (data not shown). Without being bound by theory, it was hypothesized that non-specific interactions occur at the higher protein concentrations, as HBD proteins exhibit pI values >9. Representative kinetic fits are shown in FIG. 7-FIG. 9, and the kinetic rate constants (kₒₙ, k_{off}), dissociation constants (K_{D}), and binding stoichiometries (protein molecules bound per hybrid) are provided in FIG. 19. FIG. 18B reveals that the SHBD binds the hybrid, providing a response that increases in a concentration-dependent manner, and with equilibrium binding achieved at each concentration. The rapidity of SHBD binding to and dissociation from the hybrid is evidenced by the near-vertical slopes of the sensorgram traces. Attempts were made to determine the kinetic parameters, but the values were deemed unreliable due to challenges in curve fitting, and since the k_{off} was beyond the detection limits of the instrument. While these limitations prevented accurate determination of the K_{D} based on the kinetic parameters, evaluation of the equilibrium binding as a function of the protein concentration (FIG. 18E) revealed an approach to saturation, providing a K_{D} of 8.29 μM.

A protein titration experiment using the DHBD (FIG. 18C) reveals the DHBD-hybrid complex is more stable than that of the SHBD complex. Accurate curve fitting was possible in this experiment, allowing determination of the kinetic rate parameters, and a K_{D} (k_{off}/kₒₙ) of 12 nM for the DHBD-hybrid complex. Analysis of the equilibrium binding data (FIG. 18F) yielded a K_{D} of 29 nM. The two K_{D} values are similar, and show that attaching a second HBD module by a 10aa linker provides a ˜280-fold increase in binding affinity relative to the SHBD. The binding behavior of the THBD is qualitatively and quantitatively different from that of the DHBD. The THBD on-rate is ˜4-fold faster and the off-rate is ˜14-fold slower than that of the DHBD (FIG. 19). The combination of faster on-rate and slower off-rate provides a K_{D} of 0.19 nM for the THBD-hybrid complex, and an affinity model yielded a K_{D} of 0.37 nM. Thus, attachment of a third HBD module with a 10aa linker increases hybrid affinity by 80-fold compared to the DHBD, and by over 22,000-fold compared to the SHBD. The equilibrium binding data were further analyzed to determine the stoichiometries of the complex, which are provided in FIG. 19. The non-integral values may best be interpreted by rounding upward to the nearest integral value, which would reflect the actual concentration of functionally competent protein and immobilized hybrid, both of which are expected to be less than 100 percent functional. The data indicate that, under SPR conditions, the 21 bp hairpin hybrid can bind up to six SHBDs, or two DHBDs, or one THBD.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition comprising an epiprinter molecule comprising at least one RNA:DNA hybridization domain (HBD) and at least one reactive moiety.

2. The composition of claim 1 comprising two HBDs.

3. The composition of claim 1 comprising three HBDs.

4. The composition of claim 1, wherein the epiprinter molecule comprises a sequence comprising at least 75% identity to a sequence selected from the group consisting of SEQ ID NO:2, SEQ ID NO:8, SEQ ID NO:14, SEQ ID NO:16, and SEQ ID NO:19.

5. The composition of claim 1, wherein the reactive moiety is an azide group.

6. The composition of claim 5, wherein the azide group is a side group of an amino-acid residue included in a linker sequence between two HBDs.

7. A composition comprising a nucleotide sequence encoding an epiprinter molecule of claim 1, or a fragment thereof.

8. The composition of claim 7, comprising a sequence having at least 75% identity to a sequence selected from the group consisting of SEQ ID NO:1, SEQ ID NO:5; SEQ ID NO:6, SEQ ID NO:7, SEQ ID NO:9, SEQ ID NO:10, SEQ ID NO:11, SEQ ID NO:12, SEQ ID NO:13, SEQ ID NO:15, SEQ ID NO:17, and SEQ ID NO:18.

9. The composition of claim 7, wherein the epiprinter molecule comprises a fragment comprising at least 168 nucleotides of a sequence selected from the group consisting of SEQ ID NO:1, SEQ ID NO:5; SEQ ID NO:6, SEQ ID NO:7, SEQ ID NO:9, SEQ ID NO:10, SEQ ID NO:11, SEQ ID NO:12, SEQ ID NO:15, SEQ ID NO:17, and SEQ ID NO:18.

10. A composition comprising an epitape molecule, wherein the epitape molecule comprises a DNA nanostructure comprising a structural strand of the M13 phage, mixture of oligonucleotide staples, and at least one probe strand comprising a sequence complementary to a target nucleic acid molecule of interest and at least one reactive site.

11. The composition of claim 10, wherein the epitape molecule comprises at least two probe strands.

12. The composition of claim 10, wherein the epitape molecule further comprises at least one barcode strand, wherein the barcode strand comprises a sequence that forms a dumbbell structure.

13. The composition of claim 10, wherein the at least one reactive site comprises at least one terminal alkyne on at least one probe strand.

14. The composition of claim 13, wherein the epitape comprises at least two probe sites and the at least one reactive site comprises at least one terminal alkyne on the probe strand of the second probe site.

15. The composition of claim 10, wherein the at least one probe strand is selected from the group consisting of RNA and DNA.

16. A system for detection a molecule of interest comprising at least one epiprinter molecule of claim 1, at least one epitape molecule of claim 10, wherein the epitape molecule comprises a nucleotide sequence complementary to a nucleotide sequence of the target of interest, and a nanopore detection system comprising a first reservoir containing an electrically conductive aqueous solution; an electrode disposed within the first reservoir in electrical contact with the electrically conductive aqueous solution; a second reservoir containing an electrically conductive aqueous solution; another electrode disposed within the second reservoir and in electrical contact with the electrically conductive aqueous solution;
and a membrane separating the two reservoirs, the membrane having a pore through which the epiprinter/epitape complex can pass.

17. A method for detecting the presence of target molecule of interest, the method comprising the steps of:
a) contacting the target molecule of interest with an epitape molecule comprising a probe site comprising a nucleotide sequence which is complementary to a region of the target molecule of interest;
b) contacting the target molecule:epitape complex with an epiprinter molecule;
c) allowing a cycloaddition reaction to occur between the epiprinter molecule and the epitape molecule; and
d) detecting the epiprinter:epitape complex using a nanopore system.

18. The method of claim 17, wherein the target molecule of interest is selected from the group consisting of viral nucleic acid molecules, bacterial nucleic acid molecules, a microRNA molecule, an mRNA molecule, an alternatively spliced mRNA molecule, a nucleic acid molecule harboring a disease-associated mutation, and a biomarker associated with a disease or disorder.

19. The method of claim 17, for detecting the presence of an RNA molecule of interest, the method comprising the steps of:
a) contacting an RNA molecule of interest with an epitape molecule comprising a probe site comprising a DNA sequence which is complementary to a region of the RNA molecule of interest;
b) contacting the RNA molecule:epitape complex with an epiprinter molecule;
c) allowing a cycloaddition reaction to occur between the epiprinter molecule and the epitape molecule; and
d) detecting the epiprinter:epitape complex using a nanopore system.

20. The method of claim 17, for detecting the presence of a DNA molecule of interest, the method comprising the steps of:
a) contacting a DNA molecule of interest with an epitape molecule comprising a probe site comprising a RNA sequence which is complementary to a region of the DNA molecule of interest;
b) contacting the DNA molecule:epitape complex with an epiprinter molecule;
c) allowing a cycloaddition reaction to occur between the epiprinter molecule and the epitape molecule; and
d) detecting the epiprinter:epitape complex using a nanopore system.

21. The method of claim 17, wherein the target molecule of interest is selected from the group consisting of a protein, a peptide, a chemical compound, a small molecule, a drug and a drug metabolite.

22. The method of claim 21, wherein the method comprises indirectly detecting the presence of the target molecule of interest, the method comprising the steps of:
a) contacting a target molecule of interest with a mediator complex comprising a molecule which binds specifically to the target and a nucleic acid molecule which is released upon binding of the mediator complex to the target;
b) contacting the nucleic acid molecule which was released upon binding of the mediator complex to the target with an epitape molecule comprising a probe site comprising a RNA sequence which is complementary to a region of the nucleic acid molecule which was released upon binding of the mediator complex;
c) contacting the nucleic acid molecule:epitape complex with an epiprinter molecule;
d) allowing a cycloaddition reaction to occur between the epiprinter molecule and the epitape molecule; and
e) detecting the epiprinter:epitape complex using a nanopore system.

23. The method of claim 22, wherein the mediator complex comprises at least one selected from the group consisting of an antibody, antibody fragment or aptamer specific for binding to a target molecule of interest.

24. A method of diagnosing a mammal with a disease or disorder, the method comprising the steps of:
a) detecting the presence of a nucleic acid biomarker of interest in a sample obtained from the mammal, wherein the presence of the nucleic acid biomarker of interest is associated with the disease or disorder, the method of detecting comprising:
i) contacting the sample with an epitape comprising a probe site with a probe strand comprising a nucleotide sequence complementary to a region of the biomarker of interest, wherein when the biomarker of interest is an RNA molecule, the probe strand comprises a DNA molecule, wherein when the biomarker of interest is a DNA molecule, the probe strand comprises an RNA molecule, such that the biomarker of interest hybridizes to the probe site of the epitape molecule forming an RNA:DNA hybrid;
ii) contacting the hybridized epitape: biomarker of interest with an epiprinter, whereby the epiprinter undergoes a cycloaddition reaction with the epitape molecule, becoming covalently linked to the epitape molecule;
iii) translocating the covalently linked epiprinter-epitape molecule through a nanopore, whereby the covalently linked epiprinter-epitape molecule transiently blocks the electrical signal as it passes through the nanopore; and
iv) measuring the electrical current in the nanopore system, wherein a decrease in electrical current as compared to a control indicates the presence of the biomarker of interest in the sample; and
b) diagnosing the mammal with the disease or disorder when the presence of the associated biomarker is detected.

25. The method of claim 24, wherein the target molecule of interest is selected from the group consisting of a viral nucleic acid molecule, a bacterial nucleic acid molecule, a microRNA molecule, an mRNA molecule, an alternatively spliced mRNA molecule, a nucleic acid molecule harboring a disease-associated mutation, and a biomarker associated with a disease or disorder.

26. An isolated nucleic acid molecule comprising a nucleotide sequence encoding an RNA:DNA hybrid binding molecule comprising at least one RNA:DNA hybrid binding domain, comprising a sequence having at least 75% identity to a sequence selected from the group consisting of SEQ ID NO:1, SEQ ID NO:5; SEQ ID NO:6, SEQ ID NO:7, SEQ ID NO:9, SEQ ID NO:10, SEQ ID NO:11, SEQ ID NO:12, SEQ ID NO:13, SEQ ID NO:15, SEQ ID NO:17, and SEQ ID NO:18.

27. The nucleic acid molecule of claim 26, comprising at least 168 consecutive nucleotides of a sequence selected from the group consisting of SEQ ID NO:1, SEQ ID NO:5; SEQ ID NO:6, SEQ ID NO:7, SEQ ID NO:9, SEQ ID NO:10, SEQ ID NO:11, SEQ ID NO:12, SEQ ID NO:15, SEQ ID NO:17, and SEQ ID NO:18.

28. The nucleic acid molecule of claim 26, comprising SEQ ID NO:1, SEQ ID NO:5, SEQ ID NO:7, SEQ ID NO:13, SEQ ID NO:15 or SEQ ID NO:18.

29. An RNA:DNA hybrid binding molecule comprising at least one RNA:DNA hybrid binding domain, wherein the molecule comprises an amino acid sequence selected from the group consisting of SEQ ID NO:2, SEQ ID NO:6, SEQ ID NO:8, SEQ ID NO:14, SEQ ID NO:16 and SEQ ID NO:19.

30. A method of binding at least one RNA:DNA hybrid molecule, the method comprising, contacting a sample comprising at least one RNA:DNA hybrid molecule with an RNA:DNA hybrid binding molecule of claim 29, or a nucleic acid molecule encoding an RNA:DNA hybrid binding molecule of any one of claim 26-28.
